(19) United States
(12) Patent Application Publication
(10) Pub. No.: US 2024/0401024 A1
(43) Pub. Date: Dec. 5, 2024

(54) METHODS FOR ENRICHMENT OF CIRCULAR RNA UNDER DENATURING CONDITIONS

(71) Applicant: Flagship Pioneering Innovations VI, LLC, Cambridge, MA (US)

(72) Inventors: Alexandra Sophie DE BOER, Somerville, MA (US); Nicholas McCartney PLUGIS, Duxbury, MA (US); Anthony Joseph CURA, Ayer, MA (US); Joshua Nathan FARB, Somerville, MA (US); Dineshkumar MANVAR, Andover, MA (US); Tushar Kanti MISRA, Norfolk, MA (US); Jennifer A. NELSON, Brookline, MA (US)

(21) Appl. No.: 18/718,549

(22) PCT Filed: Dec. 16, 2022
(86) PCT No.: PCT/US2022/081826

§ 371 Date: Jun. 11, 2024

Related U.S. Application Data
(60) Provisional application No. 63/291,185, filed on Dec. 17, 2021.

(51) Int. Cl. C12N 15/10; C12N 15/11

(52) U.S. Cl. CPC C12N 15/101; C12N 15/11; C12N 2310/532

(57) ABSTRACT

The present disclosure is directed to methods for the enrichment of circular polyribonucleotides (circRNA), e.g., from population of polyribonucleotides containing circRNA and linear polyribonucleotides (linRNA), where the enrichment is performed under denaturing conditions. Also disclosed are compositions including a population of polyribonucleotides containing circRNA and linRNA in a solution under denaturing conditions. Further within the scope of the present disclosure are compositions containing an enriched population of circRNA, such as a composition that was produced by exposing the composition to one or more denaturing conditions.

### BACKGROUND

Polyribonucleotides are useful for a variety of therapeutic and engineering applications. Thus, new compositions and methods for separating and purifying polyribonucleotides are useful.

### SUMMARY OF THE INVENTION

The present disclosure is directed, generally, to methods for the enrichment of circular polyribonucleotides (circRNA), e.g., from population of polyribonucleotides containing circRNA and linear polyribonucleotides (linRNA), where the enrichment is performed under denaturing conditions. Also disclosed are compositions including a population of polyribonucleotides containing circRNA and linRNA in a solution under denaturing conditions. Further within the scope of the present disclosure are compositions containing an enriched population of circRNA, such as a composition that was produced by exposing the composition to one or more denaturing conditions. The present disclosure is based, in part, on the inventors' discovery that separation of circRNA a under denaturing conditions (e.g., thermal denaturation, pH, or chemical treatment) is a robust method for purification and enrichment of circRNA from a population of mixed polyribonucleotides containing circRNA, linRNA, or other impurities or by-products. Moreover, the disclosed methods facilitate scaling up of circRNA purification processes, thereby allowing for the production and purification of large quantities of circRNA.

In an aspect, the disclosure provides a method for producing an enriched population of circRNA, the method including: (a) providing a sample including a population of polyribonucleotides including circRNA and linRNA; and (b) separating the circRNA from the linRNA under denaturing conditions that do not include the use of gel electrophoresis, thereby producing an enriched population of circRNA. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA; and (b) exposing the population of polyribonucleotides to denaturing conditions, thereby enriching the population of circRNA. Also contemplated is a method of separating a population of circRNA from a population of linRNA under the conditions described above, optionally further including a step of quantifying the population of circRNA and/or quantifying the population of linRNA.

In some embodiments, (i) the total weight of polyribonucleotides in the population of polyribonucleotides is at least 1 μg (e.g., at least 5 μg, at least 10, at least 25 μg, at least 50 μg, at least 100 μg, at least 250 μg, at least 500 μg, at least 750 μg, at least 1 mg, 5 mg, 10 mg, 100 mg, or 1 g; or between 5 μg and 100 μg, 5 μg and 500 μg, 5 μg and 1 mg, 5 μg and 10 mg, 500 μg and 100 mg, 500 μg and 1 g, 500 μg and 10 g, 100 mg and 1 g, or 1 g and 10 g); (ii) the total volume of the sample including the population of polyribonucleotides is at least 500 μL (e.g., at least 1 mL, 5 mL, 10 mL, 100 mL, or 1 L; or between 500 μL and 100 mL, 500 μL and 1 L, 500 μL and 10 L or 1L and 10 L); or (iii) the concentration of the population of polyribonucleotides in the sample is at least 200 ng/μL (e.g., at least 500 ng/μL, 1 mg/mL, 5 mg/mL, 10 mg/mL, or 50 mg/ml; or between 200 ng/μL and 100 mg/mL, 200 ng/μL and 50 mg/mL, or 1 mg/mL and 50 mg/mL). In some embodiments, the circRNA has a length from about 100 nucleotides to about 20,000 nucleotides (e.g., about 100 nucleotides to about 500 nucleotides, about 100 nucleotides to about 750 nucleotides, about 100 nucleotides to about 1,000 nucleotides, about 100 nucleotides to about 2,500 nucleotides, about 100 nucleotides to about 5,000 nucleotides, about 100 nucleotides to about 10,000 nucleotides, about 500 nucleotides to about 750 nucleotides, about 750 nucleotides to about 1,000 nucleotides, about 750 nucleotides to about 1,250 nucleotides, about 1,000 nucleotides to about 1,250 nucleotides, about 1,000 nucleotides to about 2,000 nucleotides, about 1,000 nucleotides to about 5,000 nucleotides, about 2,500 nucleotides to about 5,000 nucleotides, about 5,000 nucleotides to about 10,000 nucleotides, about 5,000 nucleotides to about 15,000 nucleotides, about 10,000 nucleotides to about 15,000 nucleotides, about 15,000 nucleotides to about 20,000 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1,000 nucleotides, about 1,250 nucleotides, about 1,500 nucleotides, about 1,750 nucleotides, about 2,000 nucleotides, about 3,000 nucleotides, about 4,000 nucleotides, about 5,000 nucleotides, about 6,000 nucleotides, about 7,000 nucleotides, about 8,000 nucleotides, about 9,000 nucleotides, about 10,000 nucleotides, about 11,000 nucleotides, about 12,000 nucleotides, about 13,000 nucleotides, about 14,000 nucleotides, about 15,000 nucleotides, about 16,000 nucleotides, about 17,000 nucleotides, about 18,000 nucleotides, about 19,000 nucleotides, or about 20,000 nucleotides). In some embodiments, the circRNA has a length of less than 1,000 nucleotides.

In an aspect, the disclosure provides a method for producing an enriched population of circRNA, the method including: (a) providing a sample including a population of polyribonucleotides including circRNA and linRNA, wherein: (i) the total weight of polyribonucleotides in the population of polyribonucleotides is at least 1 μg (e.g., at least 5 μg, at least 10, at least 25 μg, at least 50 μg, at least 100 μg, at least 250 μg, at least 500 μg, at least 750 μg, at least 1 mg, 5 mg, 10 mg, 100 mg, or 1 g; or between 5 μg and 100 μg, 5 μg and 500 μg, 5 μg and 1 mg, 5 μg and 10 mg, 500 μg and 100 mg, 500 μg and 1 g, 500 μg and 10 g, 100 mg and 1 g, or 1 g and 10 g); (ii) the total volume of the sample including the population of polyribonucleotides is at least 500 μL (e.g., at least 1 mL, 5 mL, 10 mL, 100 mL, or 1 L; or between 500 μL and 100 mL, 500 μL and 1 L, 500 L and 10 L or 1L and 10 L); or (iii) the concentration of the population of polyribonucleotides in the sample is at least 200 ng/μL (e.g., at least 500 ng/μL, 1 mg/mL, 5 mg/mL, 10 mg/mL, or 50 mg/mL; or between 200 ng/μL and 100 mg/mL, 200 ng/μL and 50 mg/mL, or 1 mg/mL and 50 mg/mL); and (b) separating the circRNA from the linRNA under denaturing conditions, thereby producing an enriched population of circRNA. Also contemplated is a method of separating a population of circRNA from a population of linRNA under the conditions described above, optionally further including a step of quantifying the population of circRNA and/or quantifying the population of linRNA.

In some embodiments, the total weight of polyribonucleotides in the population of polyribonucleotides is between 1 μg and 1000 mg (e.g., between 5 μg and 10 mg). In some embodiments, the total volume of the sample including the population of polyribonucleotides is between 500 μL and 1000 mL. In some embodiments, the concentration of the population of polyribonucleotides in the sample is between 200 ng/μL and 50 mg/mL.

In some embodiments, the circRNA has a length from about 100 nucleotides to about 20,000 nucleotides (e.g., about 100 nucleotides to about 500 nucleotides, about 100 nucleotides to about 750 nucleotides, about 100 nucleotides to about 1,000 nucleotides, about 100 nucleotides to about 2,500 nucleotides, about 100 nucleotides to about 5,000 nucleotides, about 100 nucleotides to about 10,000 nucleotides, about 500 nucleotides to about 750 nucleotides, about 750 nucleotides to about 1,000 nucleotides, about 750 nucleotides to about 1,250 nucleotides, about 1,000 nucleotides to about 1,250 nucleotides, about 1,000 nucleotides to about 2,000 nucleotides, about 1,000 nucleotides to about 5,000 nucleotides, about 2,500 nucleotides to about 5,000 nucleotides, about 5,000 nucleotides to about 10,000 nucleotides, about 5,000 nucleotides to about 15,000 nucleotides, about 10,000 nucleotides to about 15,000 nucleotides, about 15,000 nucleotides to about 20,000 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1,000 nucleotides, about 1,250 nucleotides, about 1,500 nucleotides, about 1,750 nucleotides, about 2,000 nucleotides, about 3,000 nucleotides, about 4,000 nucleotides, about 5,000 nucleotides, about 6,000 nucleotides, about 7,000 nucleotides, about 8,000 nucleotides, about 9,000 nucleotides, about 10,000 nucleotides, about 11,000 nucleotides, about 12,000 nucleotides, about 13,000 nucleotides, about 14,000 nucleotides, about 15,000 nucleotides, about 16,000 nucleotides, about 17,000 nucleotides, about 18,000 nucleotides, about 19,000 nucleotides, or about 20,000 nucleotides). In some embodiments, the circRNA has a length of less than 1,000 nucleotides.

In some embodiments, the separating step (b) is performed under denaturing conditions that do not include the use of gel electrophoresis.

In some embodiments, the enriched population of circRNA is substantially free of one or more impurities or by-products. In some embodiments, the one or more impurities or by-products include polyacrylamide, boric acid, magnesium, or ethylenediaminetetraacetic acid (EDTA).

In some embodiments, the denaturing conditions include a temperature of at least 50° C. (e.g., at least 55° C., 60° C., 65° C., 70° C., 75° C., or 80° C.). In some embodiments, the denaturing conditions include a temperature of between 50° C. and 85° C. In some embodiments, the denaturing conditions include a temperature of at least 50° C. (e.g., at least 55° C., 60° C., 65° C., 70° C., 75° C., or 80° C.) followed by a temperature of not greater than 8° C. (e.g., not greater than 7° C., 6° C., 5° C., 4° C., 3° C., 2° C., 1° C., 0° C., −1° C., −2° C., −3° C., −4° C., −5° C., −6° C., −7° C., −9° C., −10° C., −15° C., −20° C., −25° C., −30° C., −35° C., −40° C., −45° C., −50° C., −55° C., −60° C., −65° C., −70° C., −75° C., −80° C., or lower) within a time period of no greater than 30 seconds.

In some embodiments, the denaturing conditions include a pH of less than 5 (e.g., less than 4, 3, 2, or 1) or greater than 9 (e.g., greater than 10, 11, 12, or 13). In some embodiments, the denaturing conditions include a pH of less than 5 (e.g., less than 4, 3, 2, or 1). In some embodiments, the denaturing conditions include a pH of greater than 9 (e.g., greater than 10, 11, 12, or 13).

In some embodiments, the denaturing conditions include a chemical treatment. In some embodiments, the chemical treatment includes treatment with an acid, base, organic solvent, chaotropic agent, crowding agent, chelator, detergent, or salt solution.

In some embodiments, the acid includes between 1 mM and 500 mM (e.g., between 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

In some embodiments, the base includes between 1 mM and 500 mM (e.g., between 2-475 mM, mM, 60-225 mM, 70-200 mM, 80-175 mM, 90-150 mM, 100-125 mM, or 110-115 mM) sodium hydroxide, potassium hydroxide, imidazole, histidine, sodium bicarbonate, guanidine, or triethylamine.

In some embodiments, the organic solvent includes at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater; or between 0.01% and 10%) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

In some embodiments, the chaotropic agent includes between 100 mM and 8 M urea (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M), guanidinium chloride, lithium perchlorate, or polyethylene glycol (PEG). In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) n-dodecyl β-d-maltoside, n-octylglucoside, CHAPS, or deoxycholate.

In some embodiments, the crowding agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) PEG, or urea.

In some embodiments, the chelator includes between 1 mM and 10 mM (e.g., 1-2 mM, 2-3 mM, 3-4 mM, 4-5 mM, 5-6 mM, 6-7 mM, 7-8 mM, 8-9 mM, or 9-10 mM) ethylene glycol-bis(β-aminoethyl ether)-N,N,N, N′-tetra acetic acid (EGTA) or derivatives thereof, EDTA or derivatives thereof, nitrilotriacetic acid (NTA), imino-disuccinic acid (IDS), polyaspartic acid, S,S-ethylenediamine-N,N′-disuccinic acid (EDDS), or methylglycinediacetic acid (MGDA).

In some embodiments, the detergent includes between 0.005% and 0.05% (v/v) (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) Nonidet P-40 (NP40), CHAPS, octyl β-D-glucopyranoside, n-dodecyl 3-d-maltoside, Tween-20, or Tween-80.

In an aspect, the disclosure provides a method for producing an enriched population of circRNA, the method including: (a) providing a sample including a population of polyribonucleotides including circRNA and linRNA; and (b) separating the circRNA from the linRNA at a temperature of at least 50° C. (e.g., at least 55° C., 60° C., 65° C., 70° C., 75° C., 80° C., 85° C., 90° C., 95° C., or 100° C.), thereby producing an enriched population of circRNA, wherein the separating does not include the use of gel electrophoresis. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA, wherein: (i) the total mass of polyribonucleotides in the population of polyribonucleotides is at least 1 μg (e.g., at least 5 μg, at least 10, at least 25 μg, at least 50 μg, at least 100 μg, at least 250 μg, at least 500 μg, at least 750 μg, at least 1 mg, 5 mg, 10 mg, 100 mg, or 1 g; or between 5 μg and 100 μg, 5 μg and 500 μg, 5 μg and 1 mg, 5 μg and 10 mg, 500 μg and 100 mg, 500 μg and 1 g, 500 μg and 10 g, 100 mg and 1 g, or 1 g and 10 g); (ii) the total volume of the sample including the population of polyribonucleotides is at least 500 μL (e.g., at least 1 mL, 5 mL, 10 mL, 100 mL, or 1 L; or between 500 μL and 100 mL, 500 μL and 1 L, 500 μL and 10 L or 1L and 10 L); or (iii) the concentration of the population of polyribonucleotides in the sample is at least 200 ng/μL (e.g., at least 500 ng/μL, 1 mg/mL, 5 mg/mL, 10 mg/mL, or 50 mg/mL; or between 200 ng/μL and 100 mg/mL, 200 ng/μL and 50 mg/mL, or 1 mg/mL and 50 mg/mL); and (b) separating the circRNA from the linRNA at a temperature of at least 50° C. (e.g., at least 55° C., 60° C., 65° C., 70° C., 75° C., 80° C., 85° C., 90° C., 95° C., or 100° C.), thereby producing an enriched population of circRNA. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA; and (b) exposing the population of polyribonucleotides to a temperature of at least 50° C. (e.g., at least 55° C., 60° C., 65° C., 70° C., 75° C., 80° C., 85° C., 90° C., 95° C., or 100° C.), thereby enriching the population of circRNA. In some embodiments, the temperature is between 50° C. and 85° C. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA; and (b) separating the circRNA from the linRNA at a pH of less than 5 (e.g., less than 4, 3, 2, or 1) or greater than 9 (e.g., greater than 10, 11, 12, or 13), thereby producing an enriched population of circRNA, wherein the separating does not include the use of gel electrophoresis. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA, wherein: (i) the total mass of polyribonucleotides in the population of polyribonucleotides is at least 1 μg (e.g., at least 5 μg, at least 10, at least 25 μg, at least 50 μg, at least 100 μg, at least 250 μg, at least 500 μg, at least 750 μg, at least 1 mg, 5 mg, 10 mg, 100 mg, or 1 g; or between 5 μg and 100 μg, 5 μg and 500 μg, 5 μg and 1 mg, 5 μg and 10 mg, 500 μg and 100 mg, 500 μg and 1 g, 500 μg and 10 g, 100 mg and 1 g, or 1 g and 10 g); (ii) the total volume of the sample including the population of polyribonucleotides is at least 500 μL (e.g., at least 1 mL, 5 mL, 10 mL, 100 mL, or 1 L; or between 500 μL and 100 mL, 500 μL and 1 L, 500 μL and 10 L or 1L and 10 L); or (iii) the concentration of the population of polyribonucleotides in the sample is at least 200 ng/μL (e.g., at least 500 ng/μL, 1 mg/mL, 5 mg/mL, 10 mg/mL, or 50 mg/mL; or between 200 ng/μL and 100 mg/mL, 200 ng/μL and 50 mg/mL, or 1 mg/mL and 50 mg/mL); and (b) separating the circRNA from the linRNA at a pH of less than 5 (e.g., less than 4, 3, 2, or 1) or greater than 9 (e.g., greater than 10, 11, 12, or 13), thereby producing an enriched population of circRNA. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA; and (b) exposing the population of polyribonucleotides to a pH of less than 5 (e.g., less than 4, 3, 2, or 1) or greater than 9 (e.g., greater than 10, 11, 12, or 13), thereby enriching the population of circRNA. Also contemplated is a method of separating a population of circRNA from a population of linRNA under the conditions described above, optionally further including a step of quantifying the population of circRNA and/or quantifying the population of linRNA.

In some embodiments, the pH is less than 5 (e.g., less than 4, 3, 2, or 1). In some embodiments, the pH is greater than 9 (e.g., greater than 10, 11, 12, or 13).

In an aspect, the disclosure provides a method for producing an enriched population of circRNA, the method including: (a) providing a sample including a population of polyribonucleotides including circRNA and linRNA; and (b) separating the circRNA from the linRNA under conditions including an acid, base, organic solvent, chaotropic agent, crowding agent, chelator, detergent, or salt solution, thereby producing an enriched population of circRNA, wherein the separating does not include the use of gel electrophoresis. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA, wherein: (i) the total mass of polyribonucleotides in the population of polyribonucleotides is at least 1 μg (e.g., at least 5 μg, at least 10, at least 25 μg, at least 50 μg, at least 100 μg, at least 250 μg, at least 500 μg, at least 750 μg, at least 1 mg, 5 mg, 10 mg, 100 mg, or 1 g; or between 5 μg and 100 μg, 5 μg and 500 μg, 5 μg and 1 mg, 5 μg and 10 mg, 500 μg and 100 mg, 500 μg and 1 g, 500 μg and 10 g, 100 mg and 1 g, or 1 g and 10 g); (ii) the total volume of the sample including the population of polyribonucleotides is at least 500 μL (e.g., at least 1 mL, 5 mL, 10 mL, 100 mL, or 1 L; or between 500 μL and 100 mL, 500 μL and 1 L, 500 μL and 10 L or 1L and 10 L); or (iii) the concentration of the population of polyribonucleotides in the sample is at least 200 ng/μL (e.g., at least 500 ng/μL, 1 mg/mL, 5 mg/mL, 10 mg/mL, or 50 mg/mL; or between 200 ng/μL and 100 mg/mL, 200 ng/μL and 50 mg/mL, or 1 mg/mL and 50 mg/mL); and (b) separating the circRNA from the linRNA under conditions including an acid, base, organic solvent, chaotropic agent, crowding agent, chelator, detergent, or salt solution, thereby producing an enriched population of circRNA. Also contemplated is a method of separating a population of circRNA from a population of linRNA under the conditions described above, optionally further including a step of quantifying the population of circRNA and/or quantifying the population of linRNA.

In an aspect, the disclosure provides a method for producing an enriched population of circRNA, the method including: (a) providing a sample including a population of polyribonucleotides including circRNA and linRNA; and (b) exposing the population of polyribonucleotides to an acid, base, organic solvent, chaotropic agent, crowding agent, chelator, detergent, or salt solution, thereby enriching the population of circRNA. Also contemplated is a method of separating a population of circRNA from a population of linRNA under the conditions described above, optionally further including a step of quantifying the population of circRNA and/or quantifying the population of linRNA.

In some embodiments, the acid includes at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

In some embodiments, the base includes between 1 mM and 500 mM (e.g., between 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) sodium hydroxide, potassium hydroxide, imidazole, histidine, sodium bicarbonate, guanidine, or triethylamine.

In some embodiments, the organic solvent includes at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater; or between 0.01% and 10%) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) urea, guanidinium chloride, lithium perchlorate, or PEG. In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) n-dodecyl-d-maltoside, n-octylglucoside, CHAPS, or deoxycholate.

In some embodiments, the crowding agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) PEG, or urea.

In some embodiments, the chelator includes between 1 mM and 10 mM (e.g., 1-2 mM, 2-3 mM, 3-4 mM, 4-5 mM, 5-6 mM, 6-7 mM, 7-8 mM, 8-9 mM, or 9-10 mM) EGTA or derivatives thereof, EDTA or derivatives thereof, NTA, IDS, EDDS, or MGDA.

In some embodiments, the detergent includes between 0.005% and 0.05% (v/v) (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) NP40, CHAPS, octyl β-D-glucopyranoside, n-dodecyl β-d-maltoside, Tween-20, or Tween-80.

In some embodiments, step (b) includes performing column chromatography on the population of polyribonucleotides, wherein performing column chromatography includes an equilibration step, sample loading step, column washing step, and elution step.

In some embodiments, the separating is performed during the sample loading step. In some embodiments, the separating is performed during the column washing step. In some embodiments, the separating is performed during the elution step.

In some embodiments, the column chromatography includes fast protein liquid chromatography (FPLC), high-pressure liquid chromatography (HPLC), hydrophobic interaction chromatography (HIC), anion exchange chromatography (AEC), mixed mode chromatography, or affinity chromatography. In some embodiments, the AEC includes use of an anion exchange resin including of styrene-divinylbenzene, silica, Sepharose, cellulose, dextran, epoxy polyamine, methacrylate, agarose, or acrylic. In some embodiments, the anion exchange resin includes an ion exchanger including quaternary ammonium, amino ethyl, diethylaminoethyl, or diethylaminopropyl. In some embodiments, the anion exchange resin includes beads, wherein the beads have a bead diameter of 45-165 μm and a pore size of diameter 100-1000 nm. In some embodiments, the AEC includes use of a linear gradient elution or a step isocratic elution. In some embodiments, the AEC includes use of a flow rate that is between 1 mL/min and 150 mL/min.

In some embodiments, the FPLC is reversed phase-FPLC (RP-FPLC).

In some embodiments, step (b) is performed by pooling multiple fractions of purified circRNA.

In some embodiments, the circRNA and the linRNA have the same ribonucleotides sequence. In some embodiments, the circRNA and the linRNA have the same mass. In some embodiments, the circRNA and the linRNA lack a poly(A) tail.

In some embodiments, the method includes exonuclease digestion of the linRNA. In some embodiments, the method does not include exonuclease digestion of the linRNA. In some embodiments, the method does not include a selective modification to the circRNA or to the linRNA that improves enrichment of the circRNA.

In some embodiments, the percent (w/w) of the circRNA in the enriched population of circRNA is 2-fold greater than the percent (w/w) of the circRNA in the polyribonucleotide population. In some embodiments, the percent (w/w) of the circRNA in the enriched population of circRNA is at least 65% (e.g., at least 70%, 75%, 80%, 85%, 90%, 95%, 96%, 97%, 98%, or 99%). In some embodiments, the percent (w/w) of the linRNA in the enriched population of circRNA is less than 35% (e.g., less than 30%, 25%, 20%, 15%, 10%, 5%, 4%, 3%, 2%, or 1%).

In some embodiments, the circRNA has a length from about 100 nucleotides to about 20,000 nucleotides (e.g., about 100 nucleotides to about 500 nucleotides, about 100 nucleotides to about 750 nucleotides, about 100 nucleotides to about 1,000 nucleotides, about 100 nucleotides to about 2,500 nucleotides, about 100 nucleotides to about 5,000 nucleotides, about 100 nucleotides to about 10,000 nucleotides, about 500 nucleotides to about 750 nucleotides, about 750 nucleotides to about 1,000 nucleotides, about 750 nucleotides to about 1,250 nucleotides, about 1,000 nucleotides to about 1,250 nucleotides, about 1,000 nucleotides to about 2,000 nucleotides, about 1,000 nucleotides to about 5,000 nucleotides, about 2,500 nucleotides to about 5,000 nucleotides, about 5,000 nucleotides to about 10,000 nucleotides, about 5,000 nucleotides to about 15,000 nucleotides, about 10,000 nucleotides to about 15,000 nucleotides, about 15,000 nucleotides to about 20,000 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1,000 nucleotides, about 1,250 nucleotides, about 1,500 nucleotides, about 1,750 nucleotides, about 2,000 nucleotides, about 3,000 nucleotides, about 4,000 nucleotides, about 5,000 nucleotides, about 6,000 nucleotides, about 7,000 nucleotides, about 8,000 nucleotides, about 9,000 nucleotides, about 10,000 nucleotides, about 11,000 nucleotides, about 12,000 nucleotides, about 13,000 nucleotides, about 14,000 nucleotides, about 15,000 nucleotides, about 16,000 nucleotides, about 17,000 nucleotides, about 18,000 nucleotides, about 19,000 nucleotides, or about 20,000 nucleotides). In some embodiments, the circRNA has a length of less than 1,000 nucleotides.

In an aspect, the disclosure provides a composition including a population of polyribonucleotides including circRNA and linRNA, wherein the population of polyribonucleotides is in solution under denaturing conditions, and wherein: (i) the total mass of polyribonucleotides in the population of polyribonucleotides is at least 1 μg (e.g., at least 5 μg, at least 10, at least 25 μg, at least 50 μg, at least 100 μg, at least 250 μg, at least 500 μg, at least 750 μg, at least 1 mg, 5 mg, 10 mg, 100 mg, or 1 g; or between 5 μg and 100 μg, 5 μg and 500 μg, 5 μg and 1 mg, 5 μg and 10 mg, 500 μg and 100 mg, 500 μg and 1 g, 500 μg and 10 g, 100 mg and 1 g, or 1 g and 10 g); (ii) the total volume of the sample including the population of polyribonucleotides is at least 500 μL (e.g., at least 1 mL, 5 mL, 10 mL, 100 mL, or 1 L; or between 500 μL and 100 mL, 500 μL and 1 L, 500 μL and 10 L or 1L and 10 L); or (iii) the concentration of the population of polyribonucleotides in the sample is at least 500 ng/μL (e.g., at least 500 ng/μL, 1 mg/mL, 5 mg/mL, 10 mg/mL, or 50 mg/mL; or between 200 ng/μL and 100 mg/mL, 200 ng/μL and 50 mg/mL, or 1 mg/ml and 50 mg/mL).

In an aspect, the disclosure provides a composition including a population of polyribonucleotides including circRNA and linRNA, wherein the population of polyribonucleotides is in solution under denaturing conditions, and wherein, the solution is substantially free of the one or more impurities or by-products.

In an aspect, the disclosure provides a composition including a population of polyribonucleotides including circRNA and linRNA, wherein: (a) the composition is obtained from a sample including a population of nucleic acids; (b) the composition has been exposed to one or more denaturing conditions; and (c) the composition is substantially free of one or more impurities or by-products. In an aspect, the disclosure provides a composition including an enriched population of circRNA, wherein: (a) the composition is obtained from a sample including a population of polyribonucleotides including circRNA and linRNA; (b) the composition has been exposed to one or more denaturing conditions; and (c) the composition is substantially free of one or more impurities or by-products.

In some embodiments, the circRNA has a length from about 100 nucleotides to about 20,000 nucleotides (e.g., about 100 nucleotides to about 500 nucleotides, about 100 nucleotides to about 750 nucleotides, about 100 nucleotides to about 1,000 nucleotides, about 100 nucleotides to about 2,500 nucleotides, about 100 nucleotides to about 5,000 nucleotides, about 100 nucleotides to about 10,000 nucleotides, about 500 nucleotides to about 750 nucleotides, about 750 nucleotides to about 1,000 nucleotides, about 750 nucleotides to about 1,250 nucleotides, about 1,000 nucleotides to about 1,250 nucleotides, about 1,000 nucleotides to about 2,000 nucleotides, about 1,000 nucleotides to about 5,000 nucleotides, about 2,500 nucleotides to about 5,000 nucleotides, about 5,000 nucleotides to about 10,000 nucleotides, about 5,000 nucleotides to about 15,000 nucleotides, about 10,000 nucleotides to about 15,000 nucleotides, about 15,000 nucleotides to about 20,000 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1,000 nucleotides, about 1,250 nucleotides, about 1,500 nucleotides, about 1,750 nucleotides, about 2,000 nucleotides, about 3,000 nucleotides, about 4,000 nucleotides, about 5,000 nucleotides, about 6,000 nucleotides, about 7,000 nucleotides, about 8,000 nucleotides, about 9,000 nucleotides, about 10,000 nucleotides, about 11,000 nucleotides, about 12,000 nucleotides, about 13,000 nucleotides, about 14,000 nucleotides, about 15,000 nucleotides, about 16,000 nucleotides, about 17,000 nucleotides, about 18,000 nucleotides, about 19,000 nucleotides, or about 20,000 nucleotides). In some embodiments, the circRNA has a length of 1,000 nucleotides or less.

In some embodiments, the one or more impurities or by-products include polyacrylamide, boric acid, magnesium, or EDTA.

In some embodiments, the denaturing conditions include a temperature of at least 50° C. (e.g., at least 55° C., 60° C., 65° C., 70° C., 75° C., 80° C., 85° C., 90° C., 95° C., or 100° C.). In some embodiments, the denaturing conditions include a temperature of between 50° C. and 85° C. In some embodiments, the denaturing conditions include a temperature of at least 50° C. (e.g., at least 55° C., 60° C., 65° C., 70° C., 75° C., 80° C., 85° C., 90° C., 95° C., or 100° C.) followed by a temperature of not greater than 8° C. (e.g., not greater than 7° C., 6° C., 5° C., 4° C., 3° C., 2° C., 1° C., 0° C., −1° C., −2° C., −3° C., −4° C., −5° C., −6° C., −7° C., −9° C., −10° C., −15° C., −20° C., −25° C., −30° C., −35° C., −40° C., −45° C., −50° C., −55° C., −60° C., −65° C., −70° C., −75° C., −80° C., or lower).

In some embodiments, the denaturing conditions include a pH of less than 5 (e.g., less than 4, 3, 2, or 1) or greater than 9 (e.g., greater than 10, 11, 12, or 13).

In some embodiments, the denaturing conditions include a chemical treatment. In some embodiments, the chemical treatment includes treatment with an acid, base, organic solvent, chaotropic agent, crowding agent, chelator, detergent, or salt solution.

In some embodiments, the acid includes between 1 mM and 500 mM (e.g., between 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

In some embodiments, the base includes between 1 mM and 500 mM (e.g., between 2-475 mM, mM, 60-225 mM, 70-200 mM, 80-175 mM, 90-150 mM, 100-125 mM, or 110-115 mM) sodium hydroxide, potassium hydroxide, imidazole, histidine, sodium bicarbonate, guanidine, or triethylamine.

In some embodiments, the organic solvent includes at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater; or between 0.01% and 10%) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of urea, guanidinium chloride, lithium perchlorate, or PEG. In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of n-dodecyl β-d-maltoside, n-octylglucoside, CHAPS, or deoxycholate.

In some embodiments, the crowding agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) PEG, or urea.

In some embodiments, the chelator includes between 1 mM and 10 mM (e.g., 1-2 mM, 2-3 mM, 3-4 mM, 4-5 mM, 5-6 mM, 6-7 mM, 7-8 mM, 8-9 mM, or 9-10 mM) EGTA or derivatives thereof, EDTA or derivatives thereof, NTA, IDS, EDDS, or MGDA.

In some embodiments, the detergent includes between 0.005% and 0.05% (v/v) (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) NP40, CHAPS, octyl β-D-glucopyranoside, n-dodecyl β-d-maltoside, Tween-20, or Tween-80.

In some embodiments, the percent (w/w) of the circRNA in the enriched population of circRNA is 2-fold greater than the percent (w/w) of the circRNA in the polyribonucleotide population. In some embodiments, the percent (w/w) of the circRNA in the enriched population of circRNA is at least 65% (e.g., at least 70%, 75%, 80%, 85%, 90%, 95%, 96%, 97%, 98%, or 99%). In some embodiments, the percent (w/w) of the linRNA in the enriched population of circRNA is less than 35% (e.g., less than 30%, 25%, 20%, 15%, 10%, 5%, 4%, 3%, 2%, or 1%).

In some embodiments, the circRNA and the linRNA have the same ribonucleotides sequence. In some embodiments, the circRNA and the linRNA have the same mass. In some embodiments, the circRNA and the linRNA lack a poly(A) tail.

In an aspect, the disclosure provides a method of determining the purity of a circRNA, the method including: (a) providing a sample including a population of polyribonucleotides including circRNA and linRNA; (b) separating the circRNA from the linRNA under denaturing conditions by chromatography; and (c) collecting a chromatogram of the sample including a peak for the circRNA and a peak for the linRNA; (d) calculating the area under each peak to determine the purity of the circRNA in the sample.

In some embodiments, the denaturing conditions do not include the use of gel electrophoresis.

In some embodiments, the denaturing conditions include a temperature of at least 50° C. (e.g., at least 55° C., 60° C., 65° C., 70° C., 75° C., 80° C., 85° C., 90° C., 95° C., or 100° C.). In some embodiments, the denaturing conditions include a temperature of between 50° C. and 85° C. In some embodiments, the denaturing conditions include a temperature of at least 50° C. followed by a temperature of not greater than 8° C. (e.g., not greater than 7° C., 6° C., 5° C., 4° C., 3° C., 2° C., 1° C., 0° C., −1° C., −2° C., −3° C., −4° C., −5° C., −6° C., −7° C., −9° C., −10° C., −15° C., −20° C., −25° C., −30° C., −35° C., −40° C., −45° C., −50° C., −55° C., −60° C., −65° C., −70° C., −75° C., −80° C., or lower) within a time period of no greater than 30 seconds.

In some embodiments, the denaturing conditions include a pH of less than 5 (e.g., less than 4, 3, 2, or 1) or greater than 9 (e.g., greater than 10, 11, 12, or 13). In some embodiments, the denaturing conditions include a pH of less than 5 (e.g., less than 4, 3, 2, or 1). In some embodiments, the denaturing conditions include a pH of greater than 9 (e.g., greater than 10, 11, 12, or 13).

In some embodiments, the denaturing conditions include a chemical treatment. In some embodiments, the chemical treatment includes treatment with an acid, base, organic solvent, chaotropic agent, crowding agent, chelator, detergent, or salt solution.

In some embodiments, the acid includes between 1 mM and 500 mM (e.g., between 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

In some embodiments, the base includes between 1 mM and 500 mM (e.g., between 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) sodium hydroxide, potassium hydroxide, imidazole, histidine, sodium bicarbonate, guanidine, or triethylamine.

In some embodiments, the organic solvent includes at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater; or between 0.01% and 10%) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) urea, guanidinium chloride, lithium perchlorate, or PEG. In some embodiments, the chaotropic agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) n-dodecyl β-d-maltoside, n-octylglucoside, CHAPS, or deoxycholate.

In some embodiments, the crowding agent includes between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) PEG, or urea.

In some embodiments, the chelator includes between 1 mM and 10 mM (e.g., 1-2 mM, 2-3 mM, 3-4 mM, 4-5 mM, 5-6 mM, 6-7 mM, 7-8 mM, 8-9 mM, or 9-10 mM) EGTA or derivatives thereof, ethylenediaminetetraacetic acid (EDTA) or derivatives thereof, nitrilotriacetic acid (NTA), imino-disuccinic acid (IDS), polyaspartic acid, S,S-ethylenediamine-N,N′-disuccinic acid (EDDS), or methylglycinediacetic acid (MGDA).

In some embodiments, the detergent includes between 0.005% and 0.05% (v/v) (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) Nonidet P-40 (NP40), CHAPS, octyl β-D-glucopyranoside, n-dodecyl β-d-maltoside, Tween-20, or Tween-80.

In some embodiments, the chromatography includes liquid chromatography. In some embodiments, the liquid chromatography is selected from the group consisting of FPLC, HPLC, HIC, AEC, MMC, or affinity chromatography.

In some embodiments, the relative standard deviation (RSD) of the purity is less than 5% (e.g., less than 4.5%, 4%, 3.5%, 3%, 2.5%, 2%, 1.5%, 1%, or 0.5%).

### Definitions

To facilitate the understanding of this disclosure, a number of terms are defined below. Terms defined herein have meanings as commonly understood by a person of ordinary skill in the areas relevant to the disclosure.

As used herein, “a” and “an” mean “at least one” or “one or more” unless otherwise indicated. In addition, the singular forms “a,” “an,” and “the” include plural referents unless the context clearly dictates otherwise.

As used herein, “about” refers to an amount±10% of the recited value.

As used herein, the term “carrier” is a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified Phyto glycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “chromatography” refers to various chromatographic methods that can be used to purify and enrich a population of circular polyribonucleotides (circRNA) from a mixed population of polyribonucleotides containing circRNA and linear polyribonucleotides (linRNA). Chromatography includes column and non-column chromatography, for example electrophoresis methods such as capillary gel electrophoresis. Chromatography includes low or normal pressure liquid chromatography separation methods. This may include FPLC (reversed phase (RP)-FPLC and normal phase (NP)-FPLC), affinity chromatography, hydrophobic interaction chromatography, anion exchange chromatography, or mixed-mode chromatography.

As used herein, the terms “circRNA,” “circular polyribonucleotide,” and “circular RNA” are used interchangeably and mean a polyribonucleotide molecule that has a structure having no free ends (i.e., no free 3′ or 5′ ends), for example a polyribonucleotide molecule that forms a circular or end-less structure through covalent or non-covalent bonds. The circular polyribonucleotide may be, e.g., a covalently closed polyribonucleotide.

As used herein, the term “denaturing condition” refers to any condition, such as a physical or chemical condition, or set of conditions which disrupts the molecular configuration of a polynucleotide in solution. A polynucleotide under denaturing conditions may include circRNA, linRNA, linear polydeoxyribonucleotides (linDNA), or circular polydeoxyribonucleotides (circDNA). Denaturing conditions refer to conditions under which hydrogen bonding and other non-covalent forces (e.g., van der Waals forces or hydrophobic interactions) between complementary base pairs are disrupted, thereby reducing or eliminating ordered structures within the polynucleotide, such as, e.g., secondary or tertiary polymer structures (e.g., double helices, stem-loops, stacking, among others), as compared to structures observed under physiological conditions. Denaturing conditions may reduce, eliminate, or reorganize intramolecular or intermolecular interactions between nucleic acid residues of one or more polynucleotides. Denaturing conditions may also refer to conditions under which covalent bonds between contiguous nucleic acid monomers within a polymer are disrupted, such as, e.g., phosphodiester bonds between contiguous nucleosides. Without wishing to be bound by theory, circRNA may be selectively enriched in the sample relative to linRNA owing to its circular structure, which constrains the range of possible conformations and renders it less susceptible to denaturation, whereas the linRNA may be more flexible and, therefore, more amenable to disruption of secondary and tertiary structures by the denaturing conditions. Denaturing conditions may be produced in an experimental setting by manipulating one of several conditions to which the circRNA, linRNA, linDNA, circDNA, or polypeptides are exposed. Non-limiting examples of denaturing conditions are, e.g., thermal denaturation, shock-cooling, acidic or alkaline pH (e.g., less than pH 5 or greater than pH 9), or chemical treatment (e.g., with an acid, base, organic solvent, chaotropic agent, chelating agent, crowding agent, detergent, or salt solution).

As used herein, the term “eluate” refers to a fraction containing an analyte material (e.g., an enriched population of circRNA) that is eluted from a medium (e.g., a hydrophobic stationary phase) during a purification step (e.g., a chromatography step, such as, e.g., an RP-FPLC step). An eluate may be released from the medium by applying an eluent to the medium, thereby releasing the analyte. More specifically, an eluate can refer to a fraction containing circRNA that has been released from the medium following application of an eluent (e.g., an elution buffer, such as a buffer containing an organic solvent) to the medium.

As used herein, the terms “linRNA” and “linear polyribonucleotide” are used interchangeably and refer to a polyribonucleotide having a 5′ and 3′ end. In some embodiments, the linRNA has a free 5′ end or 3′ end. In some embodiments, the linRNA has non-covalently linked 5′ or 3′ ends.

As used herein, the term “modified oligonucleotide” means an oligonucleotide containing a nucleotide with at least one modification to the sugar, nucleobase, or internucleoside linkage.

As used herein, the term “modified ribonucleotide” means a ribonucleotide containing a nucleoside with at least one modification to the sugar, nucleobase, or internucleoside linkage.

As used herein, the term “naked delivery” is a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that includes a circular polyribonucleotide without covalent modification and is free from a carrier.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides, ribonucleic acids, or RNA, can refer to macromolecules that include multiple ribonucleotides that are polymerized via phosphodiester bonds. Deoxyribonucleotides are nucleotides in which the sugar is deoxyribose.

As used herein, the phrase “mixed population of polyribonucleotides” refers to a heterogenous population of polyribonucleotides. Such a heterogenous population of polyribonucleotides contains circRNA, linRNA, and, optionally, one or more impurities or by-products (e.g., one or more impurities or by-products described herein).

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used interchangeably herein, the terms “polyA” and “polyA sequence” refer to an untranslated, contiguous region of a nucleic acid molecule of at least 5 nucleotides in length and consisting of adenosine residues. In some embodiments, a polyA sequence is at least 10, at least 15, at least 20, at least 30, at least 40, or at least 50 nucleotides in length. In some embodiments, a polyA sequence is located 3′ to (e.g., downstream of) an open reason frame (e.g., an open reading frame encoding a polypeptide), and the polyA sequence is 3′ to a termination element (e.g., a Stop codon) such that the polyA is not translated. In some embodiments, a polyA sequence is located 3′ to a termination element and a 3′ untranslated region.

As used herein, the terms “purify,” “purifying,” and “purification” refer to one or more steps or processes of removing impurities or by-products (e.g., linRNA) from a sample containing a heterogenous mixture circRNA and linRNA, among other substances, to produce a composition containing an enriched population of circRNA with a reduced level of an impurity or by-product (e.g., linRNA) as compared to the original mixture or in which the linRNA or substances have been reduced by 40% or more by mass (e.g., 50%, 60%, 70%, 80%, 85%, 90%, 95%, 97%, or 99% or more) relative to a starting mixture.

As used herein, the terms “pure” and “purity” refer to the extent to which an analyte (e.g., circRNA) has been isolated and is free of other components. In the context of nucleic acids (e.g., polyribonucleotides), purity of an isolated nucleic acid (e.g., circRNA) can be expressed with regard to the population of nucleic acids that is free of any contaminants (e.g., linRNA and other substances). For example, purity of a population of circRNA indicates how much of the population is circRNA by total mass of the isolated material, which may be determined using, e.g., pure circRNA as a reference. A level of purity found in the disclosure can be 5%, 10%, 15%, 20%, 25%, 30%, 35%, 40%, 45%, 50%, 55%, 60%, 65%, 70%, 75%, 80%, 85%, 90%, 95%, greater than 95%, or greater than 99% (w/w). A “pure” population of circRNA of the disclosure can be greater than 20%, 25%, 30%, 35%, 40%, 45%, 50%, 55%, 60%, 65%, or up to 70% pure by mass. A “substantially pure” population of circRNA can be substantially free of contaminants or impurities or by-products (e.g., linRNA), e.g., greater than 70%, 75%, 80%, 85%, 90%, 95%, or >99% purity by mass. In some embodiments, the level of contaminants or impurities or by-products is no more than about 20%, 15%, 12%, 10%, 9%, 8%, 7%, 6%, 5%, 4%, 3%, 2%, or 1% (w/w). Purity can be determined by detecting a level of a specific analyte (e.g., circRNA) using gel electrophoresis, spectrophotometry (e.g., NanoDrop by ThermoFisher Scientific), or other technique suitable for measuring purity of a population of nucleic acids and calculating a percentage of the analyte (w/w) relative to the total nucleic acid content (e.g., as determined by an assay known in the art).

As used herein, the phrase “substantially free of one or more impurities or by-products” refers to a property of a sample, such as a sample containing an enriched population of circRNA, that is free of one or more impurities or by-products (e.g., one or more impurities or by-products disclosed herein) or contains a minimal amount of the one or more impurities or by-products. A minimal amount of the one or more impurities or by-products may be no more than 20% (w/w) (e.g., no more than 19%, 18%, 17%, 16%, 15%, 14%, 13%, 12%, 11%, 10%, 9%, 8%, 7%, 6%, 5%, 4%, 3%, 2%, 1% (w/w), or less). In another example, the sample or the enriched population of circRNA is substantially free of one or more impurities or by-products if the one or more impurities or by-products are present in an amount that is less than 15% (w/w) (e.g., no more than 14%, 13%, 12%, 11%, 10%, 9%, 8%, 7%, 6%, 5%, 4%, 3%, 2%, 1% (w/w), or less). In another example, the sample or the enriched population of circRNA is substantially free of one or more impurities or by-products if the one or more impurities or by-products are present in an amount that is less than 10% (w/w) (e.g., no more than 9%, 8%, 7%, 6%, 5%, 4%, 3%, 2%, 1% (w/w), or less). In another example, the sample or the enriched population of circRNA is substantially free of one or more impurities or by-products if the one or more impurities or by-products are present in an amount that is less than 5% (w/w) (e.g., no more than 4%, 3%, 2%, 1% (w/w) or less). In yet another example, the sample or the enriched population of circRNA is substantially free of one or more impurities or by-products if the one or more impurities or by-products are present in an amount that is less than 1% (no more than 0.9%, 0.8%, 0.7%, 0.6%, 0.5%, 0.4%, 0.3%, 0.2%, 0.1% (w/w), or less).

As used herein, a “termination element” is a moiety, such as a nucleic acid sequence, that terminates translation of the expression sequence in the circular or linear polyribonucleotide.

As used herein, a “translation initiation sequence” is a nucleic acid sequence that initiates translation of an expression sequence in the circular or linear polyribonucleotide.

As used herein, the term “yield” refers to the relative amount of an analyte (e.g., a population of circRNA) obtained after a purification step or process as compared to the amount of analyte in the starting material (e.g., a mixed population of polyribonucleotides, such as, e.g., circRNA and linRNA) (w/w). The yield may be expressed as a percentage. In the context of the disclosure, the amount of analyte (e.g., circRNA) in the starting material and analyte obtained after the purification step can be measured using an assay (e.g., gel electrophoresis or spectrophotometry). The methods of the disclosure can be used to produce a yield of an enriched population of circRNA of about 20% (w/w) or greater relative to the amount present in the, e.g., mixed population of polyribonucleotides or the enriched population of circRNA. For example, the methods can be used to produce a yield of purified circRNA of about 25%, 30%, 35%, 40%, 45%, 50%, 55%, 65%, 70%, 75%, 80%, 85%, or 90% (w/w) or greater.

Other features and advantages of the invention will be apparent from the following Detailed Description, the drawings, and the claims.

### BRIEF DESCRIPTION OF THE DRAWINGS

FIG. 1 shows chromatogram results for purification of circRNA without urea using a 5 mL DEAE Sepharose weak AEX column.

FIG. 2 shows chromatogram results for purification of circRNA with urea, a chaotropic salt, as the denaturing condition on a 5 mL DEAE Sepharose weak AEX column.

FIG. 3 shows chromatogram results of purification of circRNA with urea, a chaotropic salt, as the denaturing condition on an 8 mL DEAE weak AEX monolithic column.

FIG. 4 shows chromatogram results of purification of 15 mg of circRNA with urea, a chaotropic salt, as the denaturing condition on an 8 mL QA strong AEX monolithic column.

FIG. 5 shows chromatogram results of purification of 7 mg of circRNA with urea, a chaotropic salt, and pH as the denaturing condition on an 8 mL PrimaS monolithic column.

FIG. 6 shows chromatogram results following purification of circRNA with high salt and urea denaturing conditions using a 1 mL HIC monolithic column.

FIG. 7 shows gel electrophoresis results following purification of circRNA with high salt and urea denaturing conditions using a 1 mL HIC monolithic column.

FIG. 8 shows exemplary chromatogram results the separation of circRNA and linRNA following separation using temperature, chaotropic agent, and organic solvent denaturing conditions.

FIG. 9 shows HPLC analytical results following a separation of circRNA and linRNA using temperature, chaotropic agent, and organic solvent denaturing conditions.

FIG. 10 shows HPLC analytical results following a separation of circRNA and linRNa using temperature, chaotropic agent, and organic solvent denaturing conditions.

FIG. 11 shows exemplary chromatogram results following purification of circRNA with temperature and chaotropic agent conditions.

FIG. 12 shows exemplary gel electrophoresis results following purification of circRNA with temperature and chaotropic agent conditions.

FIG. 13 shows exemplary chromatogram results following purification of circRNA with high temperature and organic solvent denaturing conditions.

FIG. 14 shows exemplary chromatograms of circRNA following two water injections.

FIG. 15 shows an exemplary chromatogram of a blank injection.

FIG. 16 shows an exemplary chromatogram of circRNA and linRNA standards having separate peaks.

### DETAILED DESCRIPTION

Disclosed herein are methods for the purification and enrichment of circular polyribonucleotides (circRNA) from a heterogenous population of polyribonucleotides containing circRNA and linear polyribonucleotides (linRNA) and the purification and enrichment are performed under denaturing conditions. Also disclosed are compositions including a population of polyribonucleotides containing circRNA and linRNA in a solution under denaturing conditions, such as, e.g., such as a solution that is substantially free of one or more impurities or by-products. Further within the scope of the present disclosure are compositions containing an enriched population of circRNA, such as a composition that was produced by exposing the composition to one or more denaturing conditions. The present disclosure is based, in part, on the inventors' discovery that separation of circRNA under denaturing conditions (e.g., thermal denaturation, pH, or chemical treatment) is a robust method for purification and enrichment of circRNA from a population of mixed polyribonucleotides containing circRNA, linRNA, or other impurities or by-products, thereby improving the purification and yield of recovered circRNA relative to other methods. Moreover, the disclosed methods facilitate scaling up of circRNA purification processes, thereby allowing for the production and purification of large quantities of circRNA.

### Purification of Circular Polyribonucleotides

The present disclosure features methods of purifying and enriching circRNA from a sample containing a heterogenous population of polyribonucleotides containing circRNA and linRNA, wherein the purification is performed under denaturing conditions disclosed herein. In the context of the present disclosure, purification refers to the isolation and enrichment of a target polyribonucleotide population (e.g., circRNA) from a sample containing a mixed population of polyribonucleotides (e.g., linRNA and circRNA) as well as undesirable impurities or by-products (e.g., impurities or by-products described herein). Accordingly, following purification, circRNA is present at an increased percent (w/w) of total polyribonucleotides or at a higher concentration than in the sample from which the purified population of circRNA is obtained. Unwanted impurities or by-products in the sample may be linRNA, polyacrylamide, boric acid, magnesium, or ethylenediaminetetraacetic acid (EDTA), or any combination thereof. The disclosed methods purify and enrich circRNA from a mixed population of polyribonucleotides such that the purity of circRNA is preferably as close as possible to 100% in the enriched population of circRNA. The methods can be used to prepare an enriched population of circRNA with a purity ranging from about 5% to about 99% or greater (e.g., 5%, 10%, 15%, 20%, 25%, 30%, 35%, 40%, 45%, 50%, 55%, 60%, 65%, 70%, 75%, 80%, 85%, 90%, 95%, greater than 95%, such as 97% or 99%, or greater than 99%). In some embodiments, the purity of the enriched population of circRNA is measured as the percentage of an amount (e.g., a percentage amount) of circRNA of the enriched population relative to a percentage amount of linRNA or one or more impurities or by-products in the enriched population. For example, an enriched population of circRNA having a purity of 95% contains 95% circRNA and 5% of linRNA or one or more impurities or by-products.

In addition, the methods can be used to produce a yield of an enriched population of circRNA of about 20% (w/w) or greater relative to the amount present in the mixed population of polyribonucleotides or in the enriched population of circRNA. For example, the methods can be used to produce a yield of an enriched population of circRNA containing circRNA in an amount of about 20%, 25%, 30%, 35%, 40%, 45%, 50%, 55%, 65%, 70%, 75%, 80%, 85%, or 90% (w/w) or greater relative to the total quantity of polyribonucleotides in the enriched population. Alternatively, the enriched population of circRNA may contain circRNA in an amount of about 20%, 25%, 30%, 35%, 40%, 45%, 50%, 55%, 65%, 70%, 75%, 80%, 85%, or 90% (w/w) or greater relative to the mixed population of polyribonucleotides containing circRNA and linRNA. Further still, the enriched population of circRNA may contain circRNA in an amount of about 20%, 25%, 30%, 35%, 40%, 45%, 50%, 55%, 65%, 70%, 75%, 80%, 85%, or 90% (w/w) or greater relative to the total quantity of polynucleotides (e.g., RNA or DNA) in the enriched population or the total quantity of polynucleotides in the mixed population of polyribonucleotides.

The presently disclosed purification methods may be used in methods of preparative purification of circRNA, although the disclosed methods are also advantageous for analytical purification of circRNA. Preparative purification relates to purification of relatively large amounts of RNA. For example, preparative purification may be used to purify RNA amounts of at least 0.5 mg (e.g., at least 0.6, 0.8, 1, 2, 3, 4, 5, 6, 7, 8, 9, 10, 20, 30, 40, 50, 60, 70, 80, 90, 100, 200, 300, 400, 500, 600, 700, 800, 900, 1000 mg, or more). The benefit to this method is the ability to perform purification of circRNA in larger amounts than what is possible using other methods suitable for small-scale purification (e.g., agarose gel electrophoresis).

### Denaturing Conditions

Without wishing to be bound by theory, the present disclosure is based, in part, on the inventors' surprising discovery that purification of circRNA may be performed under denaturing conditions. Within the context of the present disclosure, denaturing conditions refer to conditions under which hydrogen bonding and other non-covalent forces (e.g., van der Waals forces or hydrophobic interactions) between complementary base pairs is disrupted, thereby reducing or eliminating ordered structures within the polynucleotide, such as, e.g., secondary or tertiary polymer structures (e.g., double helices, stem-loops, stacking, among others), as compared to structures observed under physiological conditions. Denaturing conditions also refers to conditions under which covalent bonds between contiguous nucleic acid monomers within a polymer are disrupted, such as, e.g., phosphodiester bonds between contiguous nucleosides. Furthermore, the enrichment or separation of circRNA from linRNA, among other impurities or by-products, may be improved by denaturing conditions. Such methods are particularly well-suited for scaling up or scaling out in high throughput of known RNA purification methods in the art. Accordingly, the present disclosure provides various denaturing conditions that may be used to treat a sample containing a mixed population of polyribonucleotides using a variety of purification methods, e.g., those disclosed herein.

### Thermal Denaturation

The methods disclosed herein encompass the use of high, low, or variable temperature conditions to enrich circRNA from a mixed population of polyribonucleotides containing, e.g., circRNA, linRNA, and various other impurities or by-products (e.g., salts, magnesium, urea, boric acid, etc.). For example, thermal denaturation can be performed under elevated temperature conditions, such as, e.g., a temperature of at least 50° C., at least 55° C., at least 60° C., at least 65° C., at least 70° C., at least 75° C., at least 80° C., at least 85° C., at least 90° C., at least 95° C., at least 100° C., or higher. In one example, thermal denaturation is performed under a temperature of at least 50° C. In another example, thermal denaturation is performed under a temperature of at least 50° C. but no greater than 85° C. Thermal denaturation under these temperature conditions can be performed for a time period sufficient to denature the polynucleotides (e.g., circRNA and linRNA, among others), by, e.g., disrupting intramolecular hydrogen bonding within the polynucleotides. For example, thermal denaturation can be performed at the aforementioned temperatures for a time period that is at least 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, or more. Exposure to thermal denaturing conditions can be continuous or discontinuous (e.g., 10-minute exposure to elevated temperature conditions in two 5-minute blocks separated by a time gap of, e.g., 30 seconds, 1 minute, 2 minutes, 3 minutes, 4 minutes, 5 minutes, 6 minutes, 7 minutes, 8 minutes, 9 minutes, 10 minutes, or more).

Thermal denaturation can also be performed under variable temperature conditions. For example, shock-cooling may be performed on a sample containing a heterogenous mix of polyribonucleotides including circRNA and linRNA by first exposing the sample to elevated temperature conditions (e.g., e.g., elevated temperature conditions described above) for a time period of at least 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, or more, followed by immediate exposure to low temperature conditions. Low temperature conditions may include temperatures that are not greater than 8° C. (e.g., not greater than 7° C., 6° C., 5° C., 4° C., 3° C., 2° C., 1° C., 0° C., −1° C., −2° C., −3° C., −4° C., −5° C., −6° C., −7° C., −9° C., −10° C., −15° C., −20° C., −25° C., −30° C., −35° C., −40° C., −45° C., −50° C., −55° C., −60° C., −65° C., −70° C., −75° C., −80° C., or lower). Generally, shock-cooling is performed so that the time gap between the high temperature conditions and low temperature conditions is brief. Accordingly, the time period between exposure of the sample to high temperature conditions and low temperature conditions is generally no greater than 1 minute, 55 seconds, 50 seconds, 45 seconds, 40 seconds, 35 seconds, 30 seconds, 25 seconds, 20 seconds, 15 seconds, 10 seconds, 9 seconds, 8 seconds, 7 seconds, 6 seconds, 5 seconds, 4 seconds, 3 seconds, 2 seconds, 1 second, or less. Exposure of the sample to low-temperature conditions may be for a time period that is at least 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, or more.

The aforementioned thermal denaturation protocols may be performed on a sample prior to chromatographic separation methods disclosed herein. Alternatively, thermal denaturation may be performed on a sample following chromatographic separation using the disclosed methods. Furthermore, thermal denaturation can be performed in parallel (i.e., during) chromatographic purification of the sample. For example, the sample or the mobile phase may be pre-incubated prior to loading of the sample into a chromatography column at an elevated temperature sufficient to denature intramolecular hydrogen bonds in the polyribonucleotides of the sample. In another example, thermal denaturation during chromatographic separation may be performed by placing a jacket or sleeve around the column used for the purification to achieve the desired temperature conditions sufficient for denaturation of linRNA, but not circRNA. Therefore, thermal denaturation of linRNA within the sample can be performed during any phase of the chromatographic separation process, including the equilibration step, sample loading step, column washing step, and elution step. Further still, thermal denaturation can be performed on the sample in the absence of chromatographic purification of the sample.

### pH Denaturation

According to the disclosed methods, selective purification and enrichment of circRNA from a sample containing a mixed population of polyribonucleotides that includes circRNA and linRNA by exposing the sample to denaturing pH conditions. Exemplary pH conditions include acidic conditions (e.g., pH below 7) or alkaline conditions (pH above 7), which can lead to ionization of ionizable groups within the nucleotides of the polyribonucleotides, resulting in loss of secondary structures and selective denaturation of linRNA, but not circRNA. For example, suitable pH-denaturing conditions include a pH of less than 5 (e.g., a pH of less than 4.5, 4.0, 3.5, 3.0, or lower) or greater than 9 (e.g., a pH of greater than 9.5, 10, 10.5, 11, or higher).

In one example, pH denaturation is performed at a pH of 4.9. In another example, pH denaturation is performed at a pH of 4.8. In another example, pH denaturation is performed at a pH of 4.7. In another example, pH denaturation is performed at a pH of 4.6. In another example, pH denaturation is performed at a pH of 4.5. In another example, pH denaturation is performed at a pH of 4.4. In another example, pH denaturation is performed at a pH of 4.3. In another example, pH denaturation is performed at a pH of 4.2. In another example, pH denaturation is performed at a pH of 4.1. In another example, pH denaturation is performed at a pH of 4.0. In another example, pH denaturation is performed at a pH of 3.9. In another example, pH denaturation is performed at a pH of 3.8. In another example, pH denaturation is performed at a pH of 3.8. In another example, pH denaturation is performed at a pH of 3.7. In another example, pH denaturation is performed at a pH of 3.6. In another example, pH denaturation is performed at a pH of 3.5. In another example, pH denaturation is performed at a pH of 3.4. In another example, pH denaturation is performed at a pH of 3.3. In another example, pH denaturation is performed at a pH of 3.2. In another example, pH denaturation is performed at a pH of 3.1. In yet another example, pH denaturation is performed at a pH of 3.0.

In yet another example, pH denaturation is performed at a pH of 9.1. In another example, pH denaturation is performed at a pH of 9.2. In another example, pH denaturation is performed at a pH of 9.3. In another example, pH denaturation is performed at a pH of 9.4. In another example, pH denaturation is performed at a pH of 9.5. In another example, pH denaturation is performed at a pH of 9.6. In another example, pH denaturation is performed at a pH of 9.7. In another example, pH denaturation is performed at a pH of 9.8. In another example, pH denaturation is performed at a pH of 9.9. In another example, pH denaturation is performed at a pH of 10. In another example, pH denaturation is performed at a pH of 10.1. In another example, pH denaturation is performed at a pH of 10.2. In another example, pH denaturation is performed at a pH of 10.3. In another example, pH denaturation is performed at a pH of 10.4. In another example, pH denaturation is performed at a pH of 10.5. In another example, pH denaturation is performed at a pH of 10.6. In another example, pH denaturation is performed at a pH of 10.7. In another example, pH denaturation is performed at a pH of 10.8. In yet another example, pH denaturation is performed at a pH of 10.9. In another example, pH denaturation is performed at a pH of 11.

The aforementioned pH denaturing protocols may be performed on a sample prior to chromatographic separation methods disclosed herein. Alternatively, pH denaturation may be performed on a sample following chromatographic separation using the disclosed methods. Furthermore, pH denaturation can be performed in parallel (i.e., during) chromatographic purification of the sample. For example, the sample buffers (e.g., loading buffer) used in a chromatography column may be selected or adjusted to have a pH that is sufficient to denature intramolecular hydrogen bonds in the polyribonucleotides of the sample. Accordingly, pH denaturation of linRNA within the sample can be performed during any phase of the chromatographic separation process, including the equilibration step, sample loading step, column washing step, and elution step. Further still, pH denaturation can be performed on the sample in the absence of chromatographic purification of the sample.

### Chemical Denaturation

Another method encompassed by the present disclosure for the selective enrichment and purification of circRNA from a sample containing a mixed population of polyribonucleotides that includes linRNA, circRNA, and, optionally, one or more impurities or by-products is denaturation by way of chemical treatment. Within the context of the present disclosure, denaturation by chemical treatment encompasses treatment with one or more denaturing acids, bases, organic solvents, chaotropic agents, chelating agents, crowding agent, detergents, or salt solutions.

### Acid Denaturants

Acid denaturation is a suitable method for denaturing purification methods disclosed herein on the basis that acidic solutions may protonate ionizable groups within an RNA molecule. Acids suitable for use in selective enrichment and purification of circRNA from a sample containing a mixed population of polyribonucleotides that includes linRNA, circRNA, and, optionally, one or more impurities or by-products include at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid. In some embodiments, acids suitable for use in selective enrichment and purification of circRNA from a sample containing a mixed population of polyribonucleotides that includes linRNA, circRNA, and, optionally, one or more impurities or by-products include between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

In one example, selective denaturation of linRNA, but not circRNA, is performed by exposing a sample containing a mixed population of polyribonucleotides that includes linRNA, circRNA, and, optionally, one or more impurities or by-products with a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) acetic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of acetic acid.

In another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) hydrochloric acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of hydrochloric acid.

In another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) salicylic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of salicylic acid.

In another example, acidic denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) phosphoric acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of phosphoric acid.

In another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) boric acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of boric acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) formic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of formic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) oxalic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of oxalic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) citric acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of citric acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) benzoic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of benzoic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) monochloroacetic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of monochloroacetic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) dichloroacetic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of dichloroacetic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) trichloroacetic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of trichloroacetic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) ascorbic acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of ascorbic acid.

In yet another example, acid-denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) nitric acid. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of nitric acid.

Exposure of the sample to acidic denaturing conditions may be performed for a time sufficient to denature the polyribonucleotides (e.g., linRNA or circRNA, among others). For example, exposure of the sample to acidic denaturing conditions may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, or more.

The aforementioned acidic denaturing protocols may be performed on a sample prior to chromatographic separation methods disclosed herein. Alternatively, acidic denaturation may be performed on a sample following chromatographic separation using the disclosed methods. Furthermore, acidic denaturation can be performed in parallel (i.e., during) chromatographic purification of the sample. For example, the sample buffers (e.g., loading buffer) used in a chromatography column may be include the aforementioned acids at a concentration that is sufficient to denature intramolecular hydrogen bonds in the polyribonucleotides of the sample. Accordingly, acidic denaturation of linRNA within the sample can be performed during any phase of the chromatographic separation process, including the equilibration step, sample loading step, column washing step, and elution step. Further still, acidic denaturation can be performed on the sample in the absence of chromatographic purification of the sample.

### Alkaline Denaturants

Alkaline denaturation is a suitable method for denaturing purification methods disclosed herein on the basis that alkaline solutions may deprotonate ionizable groups within an RNA molecule. Alkaline conditions suitable for use in selective enrichment and purification of circRNA from a sample containing a mixed population of polyribonucleotides that includes linRNA, circRNA, and, optionally, one or more impurities or by-products include solutions containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) sodium hydroxide, potassium hydroxide, imidazole, histidine, sodium bicarbonate, or guanidine. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of sodium hydroxide, potassium hydroxide, imidazole, histidine, sodium bicarbonate, guanidine, or triethylamine.

In one example, selective denaturation of linRNA, but not circRNA, is performed by exposing a sample containing a mixed population of polyribonucleotides that includes linRNA, circRNA, and, optionally, one or more impurities or by-products with a solution containing at least 0.5% (v/V) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) sodium hydroxide. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of sodium hydroxide.

In another example, alkaline denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) potassium hydroxide. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of potassium hydroxide.

In another example, alkaline denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) imidazole. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of imidazole.

In another example, alkaline denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) histidine. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of histidine.

In another example, alkaline denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) sodium bicarbonate. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of sodium bicarbonate.

In yet another example, alkaline denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) guanidine. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of guanidine.

In yet another example, alkaline denaturation is performed by exposing the sample to a solution containing at least 0.5% (v/v) (e.g., at least 1%, 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) triethylamine. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 mM and to 500 mM (e.g., 2-475 mM, 3-450 mM, 4-425 mM, 5-400 mM, 10-375 mM, 15-350 mM, 20-325 mM, 30-300 mM, 40-275 mM, 50-250 mM, 60-225 mM, 70-200 mM, 80-175 mM, 90-150 mM, 100-125 mM, or 110-115 mM) of triethylamine.

Exposure of the sample to alkaline denaturing conditions may be performed for a time sufficient to denature the polyribonucleotides (e.g., circRNA and linRNA, among others). For example, exposure of the sample to alkaline denaturing conditions may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, or more.

The aforementioned alkaline denaturing protocols may be performed on a sample prior to chromatographic separation methods disclosed herein. Alternatively, alkaline denaturation may be performed on a sample following chromatographic separation using the disclosed methods. Furthermore, alkaline denaturation can be performed in parallel (i.e., during) chromatographic purification of the sample. For example, the sample buffers (e.g., loading buffer) used in a chromatography column may be include the aforementioned bases at a concentration that is sufficient to denature intramolecular hydrogen bonds in the polyribonucleotides of the sample. Accordingly, alkaline denaturation of linRNA within the sample can be performed during any phase of the chromatographic separation process, including the equilibration step, sample loading step, column washing step, and elution step. Further still, alkaline denaturation can be performed on the sample in the absence of chromatographic purification of the sample.

### Organic Solvents

Organic solvents may be employed as denaturing agents suitable for use in conjunction with the disclosed methods. Organic solvents suitable for use in selective enrichment and purification of circRNA from a sample containing a mixed population of polyribonucleotides that includes linRNA, circRNA, and, optionally, one or more impurities or by-products include at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

In one example, selective denaturation of linRNA, but not circRNA, is performed by exposing a sample containing a mixed population of polyribonucleotides that includes linRNA, circRNA, and, optionally, one or more impurities or by-products with a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) dimethyl sulfoxide.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) triethylammonium acetate.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) methanol.

In another example, alkaline denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) ethanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) 2-propanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) isopropanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) butanol (e.g., 1-butanol, 2-butanol, t-butanol, or isobutanol).

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) 1-butanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) 2-butanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) t-butanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) isobutanol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) phenol.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) chloroform.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) hexane.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) acetonitrile.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) formamide.

In yet another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) propylene glycol.

Exposure of the sample to a denaturing solvent may be performed for a time sufficient to denature the polynucleotides (e.g., circRNA and linRNA, among others). For example, exposure of the sample to alkaline denaturing conditions may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, or more.

The aforementioned denaturing protocols may be performed on a sample prior to chromatographic separation methods disclosed herein. Alternatively, denaturation may be performed on a sample following chromatographic separation using the disclosed methods. Furthermore, denaturation can be performed in parallel (i.e., during) chromatographic purification of the sample. For example, the sample buffers (e.g., loading buffer) used in a chromatography column may be include the aforementioned organic solvents at a concentration that is sufficient to denature intramolecular hydrogen bonds in the polyribonucleotides of the sample. Accordingly, denaturation of linRNA within the sample can be performed during any phase of the chromatographic separation process, including the equilibration step, sample loading step, column washing step, and elution step. Further still, denaturation can be performed on the sample in the absence of chromatographic purification of the sample.

### Chaotropic Agents

Chaotropic agents (e.g., chaotropic salts) may be employed as denaturing agents suitable for use in conjunction with the disclosed methods. Chaotropic agents (e.g., chaotropic salts) suitable for use in selective enrichment and purification of circRNA from a sample containing a mixed population of polyribonucleotides that includes linRNA, circRNA, and, optionally, one or more impurities or by-products include at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) urea, guanidinium chloride, lithium perchlorate, or polyethylene glycol (PEG). In some embodiments, chaotropic agents suitable for use in selective enrichment and purification of circRNA from a sample containing a mixed population of polyribonucleotides that includes linRNA, circRNA, and, optionally, one or more impurities or by-products include between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of urea, guanidinium chloride, lithium perchlorate, or PEG.

In one example, selective denaturation of linRNA, but not circRNA, is performed by exposing a sample containing a mixed population of polyribonucleotides that includes linRNA, circRNA, and, optionally, one or more impurities or by-products with a solution containing at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) guanidinium chloride. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of urea.

In another example, denaturation is performed by exposing the sample to a solution containing at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more) lithium perchlorate. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of lithium perchlorate.

In another example, selective denaturation of linRNA, but not circRNA, is performed by exposing a sample containing a mixed population of polyribonucleotides that includes linRNA, circRNA, and, optionally, one or more impurities or by-products with a solution containing less than 1% (e.g., less than 1%, 0.9%, 0.8%, 0.7%, 0.6%, 0.5%, 0.4%, 0.3%, 0.2%, 0.1%, or less) n-dodecyl β-d-maltoside, n-octylglucoside, CHAPS, or deoxycholate. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of deoxycholate.

In one example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) n-dodecyl β-d-maltoside. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of β-d-maltoside.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) n-octylglucoside. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of n-octylglucoside.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) CHAPS. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of CHAPS.

In another example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) deoxycholate. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of deoxycholate.

Exposure of the sample to a denaturing chaotropic agent may be performed for a time sufficient to denature the polynucleotides (e.g., circRNA and linRNA, among others). For example, exposure of the sample to alkaline denaturing conditions may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes or more.

The aforementioned denaturing protocols may be performed on a sample prior to chromatographic separation methods disclosed herein. Alternatively, denaturation with a chaotropic agent may be performed on a sample following chromatographic separation using the disclosed methods. Furthermore, denaturation using chaotropic agents can be performed in parallel (i.e., during) chromatographic purification of the sample. For example, the sample buffers (e.g., loading buffer) used in a chromatography column may be include the aforementioned chaotropic agents at a concentration that is sufficient to denature intramolecular hydrogen bonds in the polyribonucleotides of the sample. Accordingly, denaturation of linRNA within the sample can be performed during any phase of the chromatographic separation process, including the equilibration step, sample loading step, column washing step, and elution step. Further still, denaturation with chaotropic agents can be performed on the sample in the absence of chromatographic purification of the sample.

### Crowding Agents

Crowding agents may be employed as denaturing agents suitable for use in conjunction with the disclosed methods on the basis that crowding agents may crowd a solution such that hydrogen bonding between other molecules may not happen. Crowding agents suitable for use in selective enrichment and purification of circRNA from a sample containing a mixed population of polyribonucleotides that includes linRNA, circRNA, and, optionally, one or more impurities or by-products include PEG and urea.

In one example, denaturation is performed by exposing the sample to a solution containing at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater) PEG. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 8 M (e.g., between 150 mM and 7.5 M, between 200 mM and 7 M, between 250 mM and 6.5 M, between 300 mM and 6 M, between 350 mM and 5.5 M, between 400 mM and 5 M, between 450 mM and 4.5 M, between 500 mM and 4 M, between 600 mM and 3.5 M, between 700 mM and 3 M, between 800 mM and 2.5 M, between 900 mM and 2 M, between 1 M and 1.5 M, between 1.1 M and 1.4 M, or between 1.2 and 1.3 M) of PEG.

PEG refers to a group of the general formula (CH₂CH₂OH)n, in which n is an integer, e.g., PEG₂-PEG₁₀₀. In some embodiments, the PEG has a molecular weight of 200 Da to 6000 Da (e.g., 400 Da to 2500 Da, 800 Da to 2200 Da, 1000 Da to 2000 Da, 200 Da, 400 Da, 600 Da, 800 Da, 1000 Da, 1200 Da, 1500 Da, 2000 Da, 2200 Da, 2500 Da, 3000 Da, 3500 Da, 4000 Da, 4500 Da, 5000 Da, 5500 Da, or 6000 Da).

The aforementioned denaturing protocols may be performed on a sample prior to chromatographic separation methods disclosed herein. Alternatively, denaturation with a crowding agent may be performed on a sample following chromatographic separation using the disclosed methods. Furthermore, denaturation using crowding agents can be performed in parallel (i.e., during) chromatographic purification of the sample. For example, the sample buffers (e.g., loading buffer) used in a chromatography column may be include the aforementioned crowding agents at a concentration that is sufficient to denature intramolecular hydrogen bonds in the polyribonucleotides of the sample.

Accordingly, denaturation of linRNA within the sample can be performed during any phase of the chromatographic separation process, including the equilibration step, sample loading step, column washing step, and elution step. Further still, denaturation with crowding agents can be performed on the sample in the absence of chromatographic purification of the sample.

### Chelating Agents

Chelating agents may be employed as denaturing agents suitable for use in conjunction with the disclosed methods on the basis that chelating agents may chelate to (i.e., bind) metal ions (e.g., Ca²⁺, Mg²⁺, Nat, and K⁺, among others) that bind to and stabilize RNA secondary and tertiary structures within the RNA molecule (e.g., a linRNA). Chelating agents suitable for use in selective enrichment and purification of circRNA from a sample containing a mixed population of polyribonucleotides that includes linRNA, circRNA, and, optionally, one or more impurities or by-products include ethylene glycol-bis(β-aminoethyl ether)-N,N, N, N-tetra acetic acid (EGTA) or derivatives thereof, ethylenediaminetetraacetic acid (EDTA) or derivatives thereof, nitrilotriacetic acid (NTA), imino-disuccinic acid (IDS), polyaspartic acid, S,S-ethylenediamine-N,N′-disuccinic acid (EDDS), or methylglycinediacetic acid (MGDA). In some embodiments, selective denaturation of linRNA, but not circRNA, is performed by exposing a sample containing a mixed population of polyribonucleotides that includes linRNA, circRNA, and, optionally, one or more impurities or by-products with a solution containing between 1 and 10 mM (e.g., 1-2 mM, 2-3 mM, 3-4 mM, 4-5 mM, 5-6 mM, 6-7 mM, 7-8 mM, 8-9 mM, or 9-10 mM) of EGTA or derivatives thereof, EDTA or derivatives thereof, NTA, IDS, EDDS, or MGDA.

In one example, selective denaturation of linRNA, but not circRNA, is performed by exposing a sample containing a mixed population of polyribonucleotides that includes linRNA, circRNA, and, optionally, one or more impurities or by-products with a solution containing EGTA or derivatives thereof. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of EGTA or derivatives thereof.

In another example, denaturation is performed by exposing the sample to a solution containing EDTA or derivatives thereof. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of EDTA or derivatives thereof.

In another example, denaturation is performed by exposing the sample to a solution containing NTA. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of NTA.

In another example, denaturation is performed by exposing the sample to a solution containing IDS. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of IDS.

In another example, denaturation is performed by exposing the sample to a solution containing polyaspartic acid. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of polyaspartic acid.

In another example, denaturation is performed by exposing the sample to a solution containing EDDS. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of EDDS.

In yet another example, denaturation is performed by exposing the sample to a solution containing MGDA. In some embodiments, the solution contains between 1 and 10 mM (e.g., 1-2 mM, 2-3 mM, 3-4 mM, 4-5 mM, 5-6 mM, 6-7 mM, 7-8 mM, 8-9 mM, or 9-10 mM) of MGDA.

Exposure of the sample to a denaturing chelating agent may be performed for a time sufficient to denature the polynucleotides (e.g., circRNA and linRNA, among others). For example, exposure of the sample to denaturing chelators may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes or more.

The aforementioned denaturing protocols may be performed on a sample prior to chromatographic separation methods disclosed herein. Alternatively, denaturation with a chelating agent may be performed on a sample following chromatographic separation using the disclosed methods. Furthermore, denaturation using chelating agents can be performed in parallel (i.e., during) chromatographic purification of the sample. For example, the sample buffers (e.g., loading buffer) used in a chromatography column may be include the aforementioned chelating agents at a concentration that is sufficient to denature intramolecular hydrogen bonds in the polyribonucleotides of the sample. Accordingly, denaturation of linRNA within the sample can be performed during any phase of the chromatographic separation process, including the equilibration step, sample loading step, column washing step, and elution step. Further still, denaturation with chelating agents can be performed on the sample in the absence of chromatographic purification of the sample.

### Detergents

Detergents may be employed as denaturing agents suitable for use in conjunction with the disclosed methods. Detergents suitable for use in selective enrichment and purification of circRNA from a sample containing a mixed population of polyribonucleotides that includes linRNA, circRNA, and, optionally, one or more impurities or by-products include Nonidet P-40 (NP40), a polysorbate (e.g., Tween-20, Tween-40, Tween-60, or Tween-80), CHAPS, octyl β-D-glucopyranoside, or n-dodecyl β-maltoside. In some embodiments, selective denaturation of linRNA, but not circRNA, is performed by exposing a sample containing a mixed population of polyribonucleotides that includes 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of NP40, a polysorbate (e.g., Tween-20, Tween-40, Tween-60, or Tween-80), CHAPS, octyl β-D-glucopyranoside, or n-dodecyl β-maltoside.

In another example, denaturation is performed by exposing the sample to a solution containing NP40. In some embodiments, denaturing is performed by exposing the sample to a solution containing 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of NP40.

In another example, denaturation is performed by exposing the sample to a solution containing Tween-20. In some embodiments, denaturing is performed by exposing the sample to a solution containing 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of Tween-20.

In another example, denaturation is performed by exposing the sample to a solution containing Tween-80. In some embodiments, denaturing is performed by exposing the sample to a solution containing 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of Tween-80.

In another example, denaturation is performed by exposing the sample to a solution containing CHAPS. In some embodiments, denaturing is performed by exposing the sample to a solution containing 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of CHAPS.

In another example, denaturation is performed by exposing the sample to a solution containing octyl β-D-glucopyranoside. In some embodiments, denaturing is performed by exposing the sample to a solution containing 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of octyl β-D-glucopyranoside.

In another example, denaturation is performed by exposing the sample to a solution containing n-dodecyl β-maltoside. In some embodiments, denaturing is performed by exposing the sample to a solution containing 0.005-0.05% (e.g., 0.006-0.045%, 0.007-0.04%, 0.008-0.035%, 0.009-0.03%. 0.01-0.025%, 0.011-0.02%, 0.012-0.019%, 0.013-0.018%, 0.014-0.017%, or 0.015-0.016%) of dodecyl β-maltoside.

Exposure of the sample to a denaturing detergent may be performed for a time sufficient to denature the polynucleotides (e.g., circRNA and linRNA, among others). For example, exposure of the sample to denaturing detergent may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, or more.

The aforementioned denaturing protocols may be performed on a sample prior to chromatographic separation methods disclosed herein. Alternatively, denaturation with a detergent may be performed on a sample following chromatographic separation using the disclosed methods. Furthermore, denaturation with detergents can be performed in parallel (i.e., during) chromatographic purification of the sample. For example, the sample buffers (e.g., loading buffer) used in a chromatography column may be include the aforementioned detergents at a concentration that is sufficient to denature intramolecular hydrogen bonds in the polyribonucleotides of the sample.

Accordingly, denaturation of linRNA within the sample can be performed during any phase of the chromatographic separation process, including the equilibration step, sample loading step, column washing step, and elution step. Further still, denaturation with detergents can be performed on the sample in the absence of chromatographic purification of the sample.

### Salt Solutions

Secondary structure formation of nucleic acid polymers is highly dependent on salt concentration, which is determinative of the free energy of base pairing and hydrogen bond formation within polynucleotides. Therefore, use of salt solutions is a suitable method for denaturing purification of polyribonucleotides, as is described herein. Salt solutions suitable for use in selective enrichment and purification of circRNA from a sample containing a mixed population of polyribonucleotides that includes linRNA, circRNA, and, optionally, one or more impurities or by-products include solutions containing, e.g., sodium chloride (NaCl), potassium chloride (KCl), magnesium chloride (MgCl), calcium chloride (CaCl), CsSO₄, NaSO₄, lithium chloride (LiCl), lithium bromide (LiBr), among other salts known in the art. In some embodiments, the salt solutions contain between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of NaCl, KCl, MgCl, CaCl, CsSO₄, NaSO₄, LiCl, or LiBr.

In one example, selective denaturation of linRNA, but not circRNA, is performed by exposing a sample containing a mixed population of polyribonucleotides that includes linRNA, circRNA, and, optionally, one or more impurities or by-products with a solution containing NaCl. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of NaCl. In another example, denaturation is performed by exposing the sample to a solution containing KCl. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of KCl.

In another example, denaturation is performed by exposing the sample to a solution containing MgCl. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of MgCl.

In another example, denaturation is performed by exposing the sample to a solution containing CaCl. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of CaCl.

In another example, denaturation is performed by exposing the sample to a solution containing CsSO₄. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of CsSO₄.

In another example, denaturation is performed by exposing the sample to a solution containing NaSO₄. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of NaSO₄.

In another example, denaturation is performed by exposing the sample to a solution containing LiCl. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of LiCl.

In another example, denaturation is performed by exposing the sample to a solution containing LiBr. In some embodiments, selective denaturation of linRNA, but not circRNA, is performed by exposing a sample containing a mixed population of polyribonucleotides that includes linRNA, circRNA, and, optionally, one or more impurities or by-products with a solution containing between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of LiBr.

Exposure of the sample to a denaturing salt solution may be performed for a time sufficient to denature the polynucleotides (e.g., circRNA and linRNA, among others). For example, exposure of the sample to denaturing salt solution may be for a time that is greater than 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, or more.

The aforementioned denaturing protocols may be performed on a sample prior to chromatographic separation methods disclosed herein. Alternatively, denaturation with a salt solution may be performed on a sample following chromatographic separation using the disclosed methods. Furthermore, denaturation with a salt solution can be performed in parallel (i.e., during) chromatographic purification of the sample. For example, the sample buffers (e.g., loading buffer) used in a chromatography column may be include the aforementioned salts at concentrations that are sufficient to denature intramolecular hydrogen bonds in the polyribonucleotides of the sample. Accordingly, denaturation of linRNA within the sample can be performed during any phase of the chromatographic separation process, including the equilibration step, sample loading step, column washing step, and elution step. Further still, denaturation with a salt solution can be performed on the sample in the absence of chromatographic purification of the sample.

### Combinations or Multiple Denaturants

The disclosure further provides use of two, three, four or more of the aforementioned denaturing conditions in combination to produce an enriched population of circRNA from a mixed population of polyribonucleotides containing circRNA, linRNA, and other substances. The sample containing the mixed population of circRNA and linRNA may be subjected to the two, three, four or more of the disclosed denaturing conditions simultaneously or sequentially (with or without temporal gaps). The two or more denaturing conditions may be from the same category of denaturants, e.g., two or more chaotropic agents selected from the chaotropic agents described herein, two or more acid denaturants selected from the acid denaturants described herein, two or more alkaline denaturants selected from the alkaline denaturants described herein, two or more organic solvents selected from the organic solvents described herein, two or more chelating agents selected from the chelating agents described herein, two or more detergents selected from the detergent described herein, or two or more salt solutions selected from the salt solutions described herein. The two or more denaturing conditions may be from different categories of denaturants. For example, the combination of two or more denaturing conditions may be as described in Table 1, wherein the thermal denaturation is selected from any thermal denaturation condition described herein, the pH denaturation is selected from any pH denaturation condition described herein, the acid denaturant is selected from any acid denaturation condition described herein, the alkaline denaturant is selected from any alkaline denaturation condition described herein, the organic solvent is selected from any organic solvent condition described herein, the chaotropic agent is selected from any chaotropic agent condition described herein, the chelating agent is selected from any chelating agent condition described herein, the detergent is selected from any detergent condition described herein, and the salt solution selected from any salt solution condition described herein.

TABLE 1

| Denaturing | | | | | | | | | |
|---|---|---|---|---|---|---|---|---|---|
| Condition | | | | | Organic | Chaotropic | Chelating | | Salt |
| Thermal | Thermal | pH | Acid | Alkaline | Solvent | Agent | Agent | Detergent | Solution |
| pH | Thermal + | | | | | | | | |
| | pH | | | | | | | | |
| Acid | Thermal + | pH + | Two or | | | | | | |
| | Acid | Acid | More | | | | | | |
| | | | Acids | | | | | | |
| Alkaline | Thermal + | pH + | Acid + | Two or | | | | | |
| | Alkaline | Alkaline | Alkaline | More | | | | | |
| | | | | Alkalizing | | | | | |
| | | | | Agents | | | | | |
| Organic | Thermal + | pH + | Acid + | Alkaline + | Two or | | | | |
| Solvent | Organic | Organic | Organic | Organic | More | | | | |
| | Solvent | Solvent | Solvent | Solvent | Organic | | | | |
| | | | | | Solvents | | | | |
| Chaotropic | Thermal + | pH + | Acid + | Alkaline + | Organic | Two or | | | |
| Agent | Chaotropic | Chaotropic | Chaotropic | Chaotropic | Solvent + | More | | | |
| | Agent | Agent | Agent | Agent | Chaotropic | Chaotropic | | | |
| | | | | | Agent | agents | | | |
| Chelating | Thermal + | pH + | Acid + | Alkaline + | Organic | Chaotropic | Two or | | |
| Agent | Chelating | Chelating | Chelating | Chelating | Solvent + | Agent + | More | | |
| | Agent | Agent | Agent | Agent | Chelating | Chelating | Chelating | | |
| | | | | | Agent | Agent | Agents | | |
| Detergent | Thermal + | pH + | Acid + | Alkaline + | Organic | Chaotropic | Chelating | Two or | |
| | Detergent | Detergent | Detergent | Detergent | Solvent + | Agent + | Agent + | More | |
| | | | | | Detergent | Detergent | Detergent | Detergents | |
| Salt | Thermal + | pH + | Acid + | Alkaline + | Organic | Chaotropic | Chelating | Detergent + | Two or |
| Solution | Salt | Salt | Salt | Salt | Solvent + | Agent + | Agent + | Salt | More |
| | Solution | Solution | Solution | Solution | Salt | Salt | Salt | Solution | Salt |
| | | | | | Solution | Solution | Solution | | Solutions |

The combinations shown in Table 1 may be combined with a third, fourth, fifth, or more further denaturing condition.

For example, denaturing thermal conditions described above may be combined with the denaturing pH conditions described above to produce an enriched population of circRNA from a mixed population of polyribonucleotides containing circRNA and linRNA. Alternatively, denaturing thermal conditions can be combined with one or more chemical denaturants described herein to produce the enriched population of circRNA. Further still, an enriched population of circRNA may be produced from a mixed population of polyribonucleotides by contacting the mixed population with a pH denaturant and a chemical denaturant.

The disclosure also provides use of one or more of denaturing conditions described herein in combination with one or more non-denaturing conditions (e.g., temperature, pH, buffers) known in the art. In some embodiments, thermal denaturation described herein may be performed on a sample containing a mixed population of polyribonucleotides containing circRNA and linRNA in combination with a denaturing pH condition. For example, the polyribonucleotides described herein (e.g., mixed population of polyribonucleotides containing circRNA and linRNA or an enriched population of circRNA) may be exposed to a denaturing temperature, such as a temperature of at least 50° C., e.g., a temperature that is between 50° C. and 85° C. (e.g., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.) in combination with exposure to denaturing pH conditions (e.g., pH that is greater than 5 and less than 9, such as a pH of, e.g., 5.01, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 8.99).

In some other embodiments, thermal denaturation described herein may be performed on a sample containing a mixed population of polyribonucleotides containing circRNA and linRNA in combination with a non-denaturing pH condition. For example, the polyribonucleotides described herein (e.g., mixed population of polyribonucleotides containing circRNA and linRNA or an enriched population of circRNA) may be exposed to a denaturing temperature, such as a temperature of at least 50° C., e.g., a temperature that is between 50° C. and 85° C. (e.g., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.) in combination with exposure to non- denaturing pH conditions (e.g., pH that is greater than 5 and less than 9, such as a pH of, e.g., 5.01, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, or 8.9).

In another example, the polyribonucleotides described herein (e.g., mixed population of polyribonucleotides containing circRNA and linRNA or an enriched population of circRNA) may be exposed to denaturing pH conditions (e.g., pH that is greater than 5 and less than 9, such as a pH of, e.g., 5.01, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 8.99) in combination with exposure to a non-denaturing temperature, such as, e.g., a temperature that is below 50° C. (e.g., 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., or less).

In another example, polyribonucleotides described herein (e.g., mixed population of polyribonucleotides containing circRNA and linRNA or an enriched population of circRNA) may be exposed to denaturing chaotropic conditions, e.g., urea, at concentrations at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more), in addition to one or more denaturing organic solvent conditions, e.g., acetonitrile and dimethyl sulfoxide, at concentrations of at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater).

In another example, polyribonucleotides described herein (e.g., mixed population of polyribonucleotides containing circRNA and linRNA or an enriched population of circRNA) may be exposed to denaturing chaotropic conditions, e.g., urea, at concentrations at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more), in addition to one or more denaturing organic solvent conditions, e.g., acetonitrile and dimethyl sulfoxide, at concentrations of at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater), in addition to a denaturing temperature, such as a temperature of at least 50° C., e.g., a temperature that is between 50° C. and 85° C. (e.g., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.).

In another example, polyribonucleotides described herein (e.g., mixed population of polyribonucleotides containing circRNA and linRNA or an enriched population of circRNA) may be exposed to denaturing chaotropic conditions, e.g., urea, at concentrations at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more), in addition to a denaturing temperature, such as a temperature of at least 50° C., e.g., a temperature that is between 50° C. and 85° C. (e.g., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.). In another example, polyribonucleotides described herein (e.g., mixed population of polyribonucleotides containing circRNA and linRNA or an enriched population of circRNA) may be exposed to denaturing chaotropic conditions, e.g., urea, at concentrations at least 1% (v/v) (e.g., at least 1.5%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 99%, or more), in addition to a denaturing pH condition (e.g., pH that is greater than 5 and less than 9, such as a pH of, e.g., 5.01, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 8.99).

In another example, polyribonucleotides described herein (e.g., mixed population of polyribonucleotides containing circRNA and linRNA or an enriched population of circRNA) may be exposed to a denaturing organic solvent conditions, e.g., acetonitrile and dimethyl sulfoxide, at concentrations of at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater), in addition to a denaturing temperature, such as a temperature of at least 50° C., e.g., a temperature that is between 50° C. and 85° C. (e.g., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., or 85° C.).

In another example, polyribonucleotides described herein (e.g., mixed population of polyribonucleotides containing circRNA and linRNA or an enriched population of circRNA) may be exposed to a denaturing organic solvent conditions, e.g., acetonitrile and dimethyl sulfoxide, at concentrations of at least 0.1% (v/v) (e.g., at least 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 40%, 50%, 60%, 70%, 75%, 80%, 90%, or greater), in addition to a denaturing pH condition (e.g., pH that is greater than 5 and less than 9, such as a pH of, e.g., 5.01, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 8.99).

In another example described herein (e.g., mixed population of polyribonucleotides containing circRNA and linRNA or an enriched population of circRNA) may be exposed to a denaturing salt conditions, e.g., NaCl, KCl, MgCl, CaCl, CsSO₄, NaSO₄, LiCl, or LiBr, at concentrations between 100 mM and 1 M (e.g., 150-950 mM, 200-900 mM, 250-850 mM, 300-800 mM, 350-750 mM, 400-700 mM, 450-650 mM, 500-600 mM, or 525-575 mM) of NaCl, KCl, MgCl, CaCl, CsSO₄, NaSO₄, LiCl, or LiBr, in addition to a denaturing pH condition (e.g., pH that is greater than 5 and less than 9, such as a pH of, e.g., 5.01, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 8.99).

Purification Methods Disclosed herein are various methods that may be used to purify circRNA from a sample containing a mixed population of circRNA and linRNA under denaturing conditions. In some embodiments, the purification method is a column chromatographic purification method. Column chromatographic methods generally employ a process in which a solution containing dissolved substances of interest, generally referred to as a mobile phase, is channeled through a chromatographic column loaded with a stationary phase made up from porous granules. The loaded solution passes through the column and substances are separated out on the basis of their interactions with the stationary phase, which is governed by the physicochemical characteristics of the analyte. Purification methods include column chromatography, such as, e.g., fast protein liquid chromatography (FPLC; such as, e.g., reversed phase (RP)-FPLC), hydrophobic interaction chromatography (HIC), anion exchange chromatography (AEC), mixed mode chromatography (MMC), or affinity chromatography. These methods are discussed in greater detail below.

### Fast Performance Liquid Chromatography

The methods described herein involve the use of FPLC methods to purify and enrich circRNA from a sample containing mixed polyribonucleotides containing one or more distinct populations of circRNA and linRNA, or other substances.

FPLC is an established method for separating complex mixtures of substances and is commonly employed in applications pertaining to biochemistry, analytical chemistry, and clinical chemistry. There are generally two types of FPLC methods, namely reversed-phase (RP)-FPLC and normal-phase (NP)-FPLC. RP-FPLC differs from NP-FPLC in that RP-FPLC employs a nonpolar (i.e., hydrophobic) stationary phase and a polar or moderately polar mobile phase, whereas NP-FPLC uses a polar (hydrophilic) stationary phase and a non-polar mobile phase. FPLC differs from high-performance liquid chromatography (HPLC) mainly by the use of lower operating pressures (e.g., <5 bar) and higher flow rates (1-5 mL/min for bench-top systems; liters/minute in industrial scale purification) as compared to HPLC. Generally, FPLC columns can only be used at a maximum pressure of up to 3-4 MPa (435-580 psi). RP-FPLC is generally performed on an apparatus that at least includes a pump with an eluent reservoir containing a mobile phase, a sample input system, a separation column containing a hydrophobic stationary phase (e.g., a resin, beads, film, among others), and a detector. Additional elements of an RP-FPLC apparatus may include a fraction collector for collecting individual fractions collected during the separation process. The core principle underlying RP-FPLC is that the interactions between a nonpolar analyte, a nonpolar stationary analytes and shorter retention and elution times for more polar analytes. This is because a decrease in the exposure of the hydrophobic segment of the analyte to the polar solvent decreases the free energy associated with the minimization of the ordered analyte-polar solvent interface. Therefore, the hydrophobicity index of an analyte is roughly proportional to its elution time from the chromatography column. The binding of the nonpolar analyte is decreased by decreasing the polarity of mobile phase, thereby leading to the elution of the analyte from the chromatography column. Other factors influencing the retention time of the analyte include presence of inorganic salts in the mobile phase, which by virtue of their effect on the mobile phase (i.e., increase in surface tension) results in increased analyte retention times. Retention time can also be influenced by the pH of the mobile phase by altering the hydrophobicity of the analyte. Generally, the mobile phase buffers are used for adjusting pH in order to neutralize any residual charge on the analyte to promote hydrophobic interactions between the analyte and the stationary phase. Similarly, the polarity of a charged analyte can be reduced by employing ion-pairing agents that neutralize analyte charge through ionic interactions.

The methods disclosed herein allow for separation of a population of circRNA from a population of mixed polyribonucleotides containing circRNA and linRNA by leveraging the differences in functional hydrophobicity between circRNA and linRNA; i.e., circRNA exhibits higher hydrophobicity as compared to linRNA, thereby leading to stronger adsorption of circRNA to the stationary phase and, consequently, longer elution times. The materials suitable for use as a reversed phase in RP-FPLC include, but are not limited to a porous polystyrene polymer, a (non-alkylated) (porous) polystyrene divinylbenzene polymer, porous silica gel, porous silica gel modified with non-polar residues, particularly porous silica gel modified with alkyl containing residues, more preferably with butyl-, octyl or octadecyl containing residues, porous silica gel modified with phenylic residues, porous polymethacrylates, wherein in particular a porous polystyrene polymer or a non-alkylated (porous) polystyrene divinylbenzene may be used. Stationary phases with polystyrene divinylbenzene are known per se. The per se known polystyrene divinyl-benzenes already used for FPLC methods, which are commercially obtainable, may be used for the method according to the invention. A non-alkylated porous polystyrene divinylbenzene which is very particularly preferred for the method according to the invention is one which, without being limited thereto, may have in particular a particle size of 8.0±1.5 μm, in particular 8.0±0.5 μm, and a pore size of 1000-1500 A, in particular 1000-1200 A or 3500-4500 A.

### Hydrophobic Interaction Chromatography

HIC is a purification method that leverages the interaction between HIC mobile phase with hydrophobic moieties of a target molecule of interest (e.g., circRNA). HIC may be performed under a bind-elute mode, in which the target molecule binds to the stationary phase until it is eluted during the elution phase, or flow-through mode, in which the molecule of interest flows through the mobile phase while the impurity or by-product binds to the stationary phase. In some embodiments, HIC may use a combination of bind-elute and flow-through modes.

HIC may employ one or more hydrophobic ligands. Non-limiting examples of HIC hydrophobic ligands include alkyl-, aryl-ligands, and combinations thereof. For example, the HIC stationary phase may be selected from the group consisting of butyl, hexyl, phenyl, octyl, or polypropylene glycol ligands. In some embodiments, the HIC column is selected from the group consisting of CaptoPhenyl, Phenyl Sepharose™ 6 Fast Flow with low or high substitution, Phenyl Sepharose™ High Performance, Octyl Sepharose™ High Performance, Fractogel™ EMD Propyl, Fractogel™ EMD Phenyl, Macro-Prep™ Methyl, Macro-Prep™ t-Butyl, WP HII-Propyl (C3)™, CIMmultus C4 HLD, CIMmultus® OH, Toyopearl™ ether, Toyopearl™ phenyl, Toyopearl™ butyl, ToyoScreen PPG, ToyoScreen Phenyl, ToyoScreen Butyl, ToyoScreen Hexyl, HiScreen Butyl FF, HiScreen Octyl FF, and Tosoh Hexyl.

According to the methods disclosed herein, HIC may employ load buffers or wash buffers that include a salt. Non-limiting examples of salts suitable for HIC include ammonium sulfate, sodium sulfate, sodium chloride, ammonium chloride, sodium bromide, or a combination thereof. In some embodiments, the load buffer and the wash buffer include a sulfate salt, a citrate salt, or a combination thereof. In various embodiments, the load buffer or the wash buffer include a cation including Ba²⁺, Ca²⁺, Mg²⁺, Li⁺, Cs⁺, Na⁺, K⁺, Rb⁺, or NH₄⁺, or an anion including PO₄ ³⁻, SO₄²⁻, CH₃CO₃⁻, Cl⁻, Br⁻, NO₃⁻, ClO₄⁻, I⁻, or SCN⁻ or a combination thereof. Hydrophobic interactions are strongest at high salt concentration. Adsorption of the population of circRNA of interest to a HIC column is favored by high salt concentrations, but the actual concentrations can vary over a wide range depending on the nature of the circRNA of interest, salt type and the particular HIC ligand chosen. In some embodiments, the salt concentration is in the range of, for example, about 50 mM to about 5000 mM, about 100 mM to about 4000 mM, about 1000 mM to about 4000 mM, about 50 mM to about 2000 mM, depending, in part, on the salt type and HIC adsorbent. In one embodiment the salt concentration is about 50 mM, about 55 mM, about 60 mM, about 65 mM, about 70 mM, about 75 mM, about 80 mM, about 85 mM, about 90 mM, about 100 mM, about 200 mM, about 300 mM, about 400 mM, about 500 mM, about 600 mM, about 700 mM, about 800 mM, about 900 mM, about 1000 mM, about 1200 mM, about 1400 mM, about 1600 mM, about 1800 mM or about 2000 mM. In some embodiments, the load buffer and the wash buffer have a pH between about 4.0 and 8.5 or between about 5.0 and 7.0. In certain embodiments, the load buffer and the wash buffer have a pH of about 4.0, about 4.5, about 5.0, about 5.5, about 6, about 6.5, about 7.0, about 7.5, about 8.0, or about 8.5. In some embodiments, the load buffer and the wash buffer are the same or substantially the same.

In an exemplary method of performing HIC separation, the sample (e.g., a sample containing a mixed population of polyribonucleotides, such as, e.g., circRNA and linRNA) is contacted with the HIC media, e.g., using a batch purification technique or using a column or membrane chromatography or monolithic material (referred to as HIC media or resin). For example, in the context of chromatographic separation, a chromatographic apparatus, commonly cylindrical in shape, is employed to contain the chromatographic support media (e.g., HIC media) prepared in an appropriate buffer solution. Once the chromatographic material is added to the chromatographic apparatus, a sample containing the population of circRNA of interest is contacted to the chromatographic material in the presence of a loading buffer to allow binding of the circRNA of interest or a substantial portion of the impurity or by-product to the HIC media. The population of circRNA of interest in the sample binds to the HIC media, while impurities or by-products, such as, e.g., linRNA, flows through, forming a flow through fraction containing the impurities or by-products. The bound circRNA may then be eluted during an elution phase, thereby producing an enriched population of circRNA. In some embodiments, the population of circRNA that binds to the HIC media is at least about 20%, at least about 30%, at least about 40%, at least about 50%, at least about 60%, at least about 70%, at least about 80%, at least about 90%, at least about 95% or at least about 98% of the total amount polyribonucleotides in the sample.

### Anion Exchange Chromatography

AEC separates molecules based on differences between the local charges of the nucleic acids of interest (e.g., circRNA) and the local charges of the chromatographic material. A packed AEC column or membrane device can be operated in a bind-elute mode, a flow-through, or a hybrid mode. After washing the column or the membrane device with the equilibration buffer or another buffer with different pH or conductivity, the product recovery is achieved by increasing the ionic strength (i.e., conductivity) of the elution buffer to compete with the solute for the charged sites of the AEC matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate(S). Cellulose ion exchange medias such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow, and Capto™ S are all available from GE Healthcare. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa., CIMmultus® QA, CIMmultus® DEAE, CIMmultus® EV, CIMmultus® EDA, and CIMmultus® SO3 from Sartorius, or Nuvia S and UNOSphere™ S from BioRad, Hercules, Calif., Eshmuno@ S from EMD Millipore, Billerica, Calif.

### Mixed Mode Chromatography

MMC is chromatography that utilizes a mixed mode media, such as, but not limited to CaptoAdhere available from GE Healthcare, CIMmultus® PrimaS, and CIMmultus® H-Bond from Sartorius. Such a media includes a MMC ligand. In some embodiments, such a ligand refers to a ligand that is capable of providing at least two different, but co-operative, sites which interact with the substance to be bound. One of these sites gives an attractive type of charge-charge interaction between the ligand and the nucleic acid of interest. The other site typically gives electron acceptor-donor interaction or hydrophobic or hydrophilic interactions. Electron donor-acceptor interactions include interactions such as hydrogen-bonding, π-π, cation-π, charge transfer, dipole-dipole, induced dipole, among others. The mixed mode functionality can give a different selectivity compared to traditional anion exchangers. MMC ligands are also known as “multimodal” chromatography ligands.

According to the present disclosure the MMC media may include mixed mode ligands coupled to an organic or inorganic support, sometimes denoted a base matrix, directly or via a spacer. The support may be in the form of particles, such as essentially spherical particles, a monolith, filter, membrane, surface, capillaries, among others. In certain embodiments, the support is prepared from a native polymer, such as cross-linked carbohydrate material, such as agarose, agar, cellulose, dextran, chitosan, konjac, carrageenan, gellan, alginate, among others. To obtain high adsorption capacities, the support can be porous, and ligands are then coupled to the external surfaces as well as to the pore surfaces. Such native polymer supports can be prepared according to standard methods, such as inverse suspension gelation (S Hjerten: BIOCHIM BIOPHYS ACTA 79 (2), 393-98 (1964). Alternatively, the support can be prepared from a synthetic polymer, such as cross-linked synthetic polymers, e.g., styrene or styrene derivatives, divinylbenzene, acrylamides, acrylate esters, methacrylate esters, vinyl esters, vinyl amides, among others. Such synthetic polymers can be produced according to standard methods, see e.g., “Styrene based polymer supports developed by suspension polymerization” (R Arshady: Chimica e L′Industria 70 (9), 70-75 (1988)). Porous native or synthetic polymer supports are also available from commercial sources, such as Amersham Biosciences, Uppsala, Sweden.

### Assessment of Purity

Assessment of purity of an enriched population of circRNA may be performed by methods well-known in the art. In an embodiment, a circRNA is at least 30%, 40%, 50%, 60%, 70%, 80%, 85%, 90%, 91%, 92%, 93%, 94%, 95%, 96%, 97%, 98%, or 99% pure on a mass basis. Purity may be measured, e.g., by mass spectrometry, HPLC, chip-based electrophoresis, microscopy, circular dichroic (CD) spectroscopy, spectrophotometry, fluorometry (e.g., Qubit), polyacrylamide gel electrophoresis imaging, UV-Vis spectrophotometry, RNA electrophoresis, RNAse H analysis, or any combination thereof. In some embodiments, at least 30%, 40%, 50%, 60%, 70%, 80%, 85%, 90%, 91%, 92%, 93%, 94%, 95%, 96%, 97%, 98%, or 99% of the total of mass of polyribonucleotide in a preparation described herein is contained in circRNA molecules.

In some embodiments, a circRNA preparation includes less than a threshold amount (e.g., where the threshold amount is a reference criterion, e.g., a pharmaceutical release specification, for the circRNA preparation) of linRNA molecules when measured by mass spectrometry, HPLC, chip-based electrophoresis, microscopy, CD spectroscopy, spectrophotometry, fluorometry (e.g., Qubit), polyacrylamide gel electrophoresis imaging, UV-Vis spectrophotometry, RNA electrophoresis, or RNAse H analysis. In some embodiments, a circRNA preparation includes an undetectable level of linRNA molecules.

In an embodiment, a circRNA (e.g., a circRNA pharmaceutical preparation or composition or an intermediate in the production of the circRNA) has a mononucleotide content of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/mL, 400 ng/ml, 500 ng/ml, 1000 μg/mL, 5000 μg/mL, 10,000 μg/mL, or 100,000 μg/mL.

In an embodiment, a circRNA has mononucleotide content less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, or any percentage therebetween of total nucleotides on a mass basis, where total nucleotide content is the total mass of deoxyribonucleotide molecules and ribonucleotide molecules.

In an embodiment, a circRNA (e.g., a circRNA pharmaceutical preparation or composition or an intermediate in the production of the circRNA) has a linRNA content, e.g., linRNA counterpart or RNA fragments, of less than 1 ng/ml, 5 ng/ml, 10 ng/ml, 15 ng/ml, 20 ng/ml, 25 ng/ml, 30 ng/mL, 35 ng/mL, 40 ng/mL, 50 ng/mL, 6 0 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 mg/mL, 1.5 mg/mL, or 2 mg/mL.

In an embodiment, a circRNA (e.g., a circRNA pharmaceutical preparation or composition or an intermediate in the production of the circRNA) has less than 0.1%, 1%, 2%, 3%, 4%, 5%, 6%, 7%, 8%, 9%, 10%, 15%, 20%, 25%, 30%, 35%, 40%, 45%, or 50% of linRNA.

In an embodiment, a circRNA (e.g., a circRNA pharmaceutical preparation or composition or an intermediate in the production of the circRNA) has a DNA content, e.g., template DNA, e.g., cell DNA (e.g., host cell DNA), of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/mL, 100 ng/mL, 200 ng/mL, 300 ng/ml, 400 ng/ml, 500 ng/mL, 1000 μg/mL, 5000 μg/mL, 10,000 μg/mL, or 100,000 μg/mL.

In an embodiment, a circRNA (e.g., a circRNA pharmaceutical preparation or composition or an intermediate in the production of the circRNA) has DNA content less than 0.1%, 1%, 2%, 3%, 4%, 5%, 6%, 7%, 8%, 9%, 10%, 15%, 20%, 25%, 30%, 35%, 40%, 45%, 50% of total nucleotides on a mass basis, wherein total nucleotide molecules is the total mass of deoxyribonucleotide content and ribonucleotide molecules.

In an embodiment, a circRNA (e.g., a circRNA pharmaceutical preparation or composition or an intermediate in the production of the circRNA) has a protein (e.g., cell protein (CP), e.g., enzyme) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/mL, 400 ng/ml, or 500 ng/mL.

In an embodiment, a circRNA (e.g., a circRNA pharmaceutical preparation or composition or an intermediate in the production of the circRNA) has a protein (e.g., CP, e.g., enzyme) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circRNA.

In an embodiment, a circRNA (e.g., a circRNA pharmaceutical preparation or composition or an intermediate in the production of the circRNA) has a circRNA concentration of at least 0.1 ng/ml, 0.5 ng/mL, 1 ng/ml, 5 ng/ml, 10 ng/ml, 50 ng/ml, 0.1 μg/mL, 0.5 μg/mL, 1 g/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1000 μg/mL, 5000 μg/mL, 10,000 μg/mL, or 100,000 μg/mL.

In some embodiments, a circRNA is purified by chromatography, e.g., liquid chromatography, e.g., FPLC (e.g., normal phase or reversed phase FPLC), HPLC, HIC, AEC, MMC, or affinity chromatography.

### Preparative Methods

In some embodiments, any conditions described herein may be used as preparative methods of circRNA from a mixed population of polyribonucleotides containing circRNA and linRNA or an enriched population of circRNA.

In some embodiments, preparative methods of producing circRNA amounts of at least 0.5 mg (e.g., at least 0.6, 0.8, 1, 2, 3, 4, 5, 6, 7, 8, 9, 10, 20, 30, 40, 50, 60, 70, 80, 90,100,200, 300, 400, 500, 600, 700, 800, 900, 1000 mg, or more). The present methods are advantageous in that they are highly scalable.

### Analytical Methods

In some embodiments, any conditions described herein may be used as analytical methods for determining the purity of circRNA from a mixed population of polyribonucleotides containing circRNA and linRNA or an enriched population of circRNA. The present invention includes methods for repeatedly determining the purity of batches of enriched circRNA using one or more denaturing conditions described herein.

In some embodiments, the analytical methods determine the purity of the circRNA using chromatography, e.g., liquid chromatography, e.g., FPLC (e.g., normal phase or reversed phase FPLC), HPLC, HIC, AEC, MMC, or affinity chromatography.

In some embodiments, the present analytical methods have relative standard deviation (RSD) of less than 50%, 45%, 40%, 35%, 30%, 25%, 20%, 15%, 10%, 9%, 8%, 7%, 6%, 5%, 4%, 3%, 2%, 1%, 0.5%, or 0.1%. In some embodiments, the RSD is calculated based on 2, 3, 4, 5, 6, 7, 8, 9, 10, 11, 12, 13, 14, 15, 16, 17, 18, 19, or 20 circRNA samples.

In some embodiments, the analytical methods include the use of mobile phases. In some embodiments, the mobile phases include urea, bis-tris propane (BTP), acetonitrile, NaCl, water, hydrochloric acid (HCl), or DMSO.

In some embodiments, the denaturing conditions are found in the mobile phases.

In some embodiments, the chromatography column is heated to provide denaturation. In some embodiments, the circRNA sample is heated before placement in the column for denaturation.

In some embodiments, the circRNA sample is denatured with a combination of denaturants such as described above, e.g., a combination of urea and acetonitrile.

In some embodiments, the analytical methods include the step of determining an impurity or by-product level.

In some embodiments, analytical methods include quantifying the purity of circRNA amounts of at least 0.5 mg (e.g., at least 0.6, 0.8, 1, 2, 3, 4, 5, 6, 7, 8, 9, 10, 20, 30, 40, 50, 60, 70, 80, 90,100,200, 300, 400, 500, 600, 700, 800, 900, 1000 mg, or more).

### Impurities and By-Products

The methods of the disclosure are suitable for generating a preparation including a population of circRNA and having a reduced level of impurity or by-product, for example, as compared to the levels of the impurity or by-product in the sample prior to purification by the methods of the present disclosure or as compared to the levels of impurity or by-product in a different sample containing 100% (w/w) circRNA. In some embodiments, the methods of the invention generate a composition having a population of circRNA of interest and having a reduced level of total impurity or by-product. In some embodiments, a preparation having a reduced level of total impurity or by-product is free of impurities or by-products or substantially free of impurities or by-products. For example, a low impurity or by-product composition may contain about 10%, 9%, 8%, 7%, 6%, 5%, 4%, 3%, 2%, 1.5%, 1.4%, 1.3%, 1.2%, 1.1%, 1%, 0.5%, or less total impurities or by-products. In some embodiments, a low impurity or by-product composition includes about 5%, 4%, 3%, 2.5%, 2.4%, 2.3%, 2.2%, 2.1%, 2%, 1.5%, 1.4%, 1.3%, 1.2%, 1.1%, 1%, 0.5%, 0.1%, or less total impurities or by-products.

In some embodiments, the impurity or by-product is a process-related impurity or by-product. As used herein, the term “process-related impurity or by-product,” refers to impurities or by-products that are present in a composition including a population of circRNA of interest but are not derived from the circRNA itself. Process-related impurities or by-products include, but are not limited to, host cell proteins (HCPs), host cell nucleic acids, chromatographic materials, and media components. For example, process-related impurities or by-products may include polyacrylamide, boric acid, magnesium, or EDTA. A “low impurity composition,” as used herein, refers to a composition including reduced levels of process-related impurities or by-products as compared to a composition wherein the impurities or by-products were not reduced. For example, a low process-related impurity composition may contain about 10%, 9%, 8%, 7%, 6%, 5%, 4%, 3%, 2%, 1%, 0.5%, or less process-related impurities or by-products. In one embodiment, a low process-related impurity composition is free of process-related impurities or by-products or is substantially free of process-related impurities or by-products.

In some embodiments, a circRNA (e.g., a circRNA pharmaceutical preparation or composition or an intermediate in the production of the circRNA) is substantially free of an impurity or by-product. In various embodiments, the level of at least one impurity or by-product in a composition including the circRNA is reduced by at least 30%, at least 40%, at least 50%, at least 60%, at least 70%, at least 80%, at least 90%, or at least 95% of a composition prior to purification or treatment to remove the impurity or by-product. In some embodiments, the level of at least one process-related impurity or by-product is reduced by at least 30%, at least 40%, at least 50%, at least 60%, at least 70%, at least 80%, at least 90%, or at least 95% of a composition prior to purification or treatment to remove the impurity or by-product. In some embodiments, the level of at least on product-related substance is reduced by at least 30%, at least 40%, at least 50%, at least 60%, at least 70%, at least 80%, at least 90%, or at least 95% of a composition prior to purification or treatment to remove the impurity or by-product.

Methods well-known in the art may be used for quantification of impurities or by-products present in a sample. For example, spectroscopy methods such as ultraviolet (UV), near-infrared spectroscopy (NIR), Fourier-transform infrared spectroscopy (FTIR), fluorescence spectroscopy, or Raman spectroscopy may be used to monitor levels of impurities or by-products in an on-line, at-line, or in-line mode. In some embodiments, on-line, at-line, or in-line monitoring methods can be used either on the wash line of the chromatography step or in the collection vessel, to enable achievement of the desired circRNA quality/recovery. In some embodiments, the UV signal can be used as a surrogate to achieve an appropriate product quality/recovery, wherein the UV signal can be processed appropriately, including, but not limited to, such processing techniques as integration, differentiation, moving average, such that normal process variability can be addressed, and the target product quality can be achieved. In some embodiments, such measurements can be combined with in-line dilution methods such that ion concentration/conductivity of the load/wash can be controlled by feedback, thereby facilitating product quality control.

### Production of Circular Polyribonucleotides

The disclosure provides methods for producing circRNA, including, e.g., recombinant technology or chemical synthesis. For example, a DNA molecule used to produce an RNA circle can include a DNA sequence of a naturally occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

The circRNA may be prepared according to any available technique, including, but not limited to chemical synthesis and enzymatic synthesis. In some embodiments, a linear primary construct or linear RNA may be cyclized or concatenated to create a circRNA described herein. The mechanism of cyclization or concatenation may occur through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, can be used for splint ligation. According to this method, a single stranded polynucleotide (splint), such as a single-stranded DNA or RNA, can be designed to hybridize with both termini of a linRNA, so that the two termini can be juxtaposed upon hybridization with the single-stranded splint. Splint ligase can thus catalyze the ligation of the juxtaposed two termini of the linRNA, generating a circRNA. In some embodiments, a DNA or RNA ligase may be used in the synthesis of the circular polynucleotides. As a non-limiting example, the ligase may be a circ ligase or circular ligase.

In another example, either the 5′ or 3′ end of the linRNA can encode a ligase ribozyme sequence such that during in vitro transcription, the resultant linear circRNA includes an active ribozyme sequence capable of ligating the 5′ end of the linRNA to the 3′ end of the linRNA. The ligase ribozyme may be derived from the Group I Intron, Hepatitis Delta Virus, Hairpin ribozyme or may be selected by SELEX (systematic evolution of ligands by exponential enrichment).

In another example, a linRNA may be cyclized or concatenated by using at least one non-nucleic acid moiety. For example, the at least one non-nucleic acid moiety may react with regions or features near the 5′ terminus or near the 3′ terminus of the linRNA in order to cyclize or concatenate the linRNA. In another example, the at least one non-nucleic acid moiety may be located in or linked to or near the 5′ terminus or the 3′ terminus of the linRNA. The non-nucleic acid moieties may be homologous or heterologous. As a non-limiting example, the non-nucleic acid moiety may be a linkage such as a hydrophobic linkage, ionic linkage, a biodegradable linkage or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, linRNAs may be cyclized or concatenated by self-splicing. In some embodiments, the linRNA may include loop E sequence to self-ligate. In another embodiment, the linRNA may include a self-circularizing intron, e.g., a 5′ and 3′ slice junction, or a self-circularizing catalytic intron such as a Group I, Group II or Group III Introns. Nonlimiting examples of group I intron self-splicing sequences may include self-splicing permuted intron-exon sequences derived from T4 bacteriophage gene td, and the intervening sequence (IVS) rRNA of Tetrahymena, cyanobacterium Anabaena pre-tRNA-Leu gene, or a Tetrahymena pre-rRNA.

In some embodiments, the polyribonucleotide may include catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phosphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In another example, a linRNA may be cyclized or concatenated by a non-nucleic acid moiety that causes an attraction between atoms, molecular surfaces at, near, or linked to the 5′ and 3′ ends of the linRNA. The one or more linRNAs may be cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linRNA may include a ribozyme RNA sequence near the 5′ terminus and near the 3′ terminus. The ribozyme RNA sequence may covalently link to a peptide when the sequence is exposed to the remainder of the ribozyme. The peptides covalently linked to the ribozyme RNA sequence near the 5′ terminus and the 3 ‘terminus may associate with each other, thereby causing a linRNA to cyclize or concatenate. In another example, the peptides covalently linked to the ribozyme RNA near the 5’ terminus and the 3′ terminus may cause the linear primary construct or linear mRNA to cyclize or concatenate after being subjected to ligated using various methods known in the art such as, but not limited to, protein ligation. Non-limiting examples of ribozymes for use in the linear primary constructs or linRNA of the present invention or a non-exhaustive listing of methods to incorporate or covalently link peptides are described in US patent application No. US20030082768, the contents of which is here in incorporated by reference in its entirety.

In yet another example, chemical methods of circularization may be used to generate the circRNA. Such methods may include, but are not limited to click chemistry (e.g., alkyne and azide-based methods, or clickable bases), olefin metathesis, phosphoramidate ligation, hemiaminal-imine crosslinking, base modification, or any combination thereof.

In another example, circular polyribonucleotide is produced using a deoxyribonucleotide template transcribed in a cell-free system (e.g., by in vitro transcription) to produce a linear RNA. The linear polyribonucleotide produces a splicing-compatible polyribonucleotide, which may be self-spliced to produce a circular polyribonucleotide.

In some embodiments, a circular polyribonucleotide is produced (e.g., in a cell-free system) by providing a linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, a circular polyribonucleotide is produced by providing a deoxyribonucleotide encoding a linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, a circular polyribonucleotide is produced by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circRNA). In some embodiments, the linear polyribonucleotide comprises a 5′ annealing region and a 3′ annealing region.

In some embodiments, the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

In another example, a circular polyribonucleotide may be produced in a cell, e.g., a prokaryotic cell or a eukaryotic cell. In some embodiments, an exogenous polyribonucleotide is provided to a cell (e.g., a linear polyribonucleotide described herein or a DNA molecule encoding for the transcription of a linear polyribonucleotide described here). The linear polyribonucleotide may be transcribed in the cell from an exogenous DNA molecule provided to the cell. The linear polyribonucleotide may be transcribed in the cell from an exogenous recombinant DNA molecule transiently provided to the cell. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein may be a bacterial cell or an archaeal cell. For example, the prokaryotic cell including the polyribonucleotides described herein may be E coli, halophilic archaea (e.g., Haloferax volcaniii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

The cell may be a eukaryotic cell. In some embodiments, the eukaryotic cell is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp, and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell may be dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell may be dedifferentiated. In some embodiments, the unicellular plant cell is from a plant callus. In some embodiments, the unicellular cell is a plant cell protoplast. In some embodiments, the unicellular eukaryotic cell is a unicellular eukaryotic algal cell, such as a unicellular green alga, a diatom, a euglenid, or a dinoflagellate. Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In some embodiments, the eukaryotic cell is a cell of an arthropod (e.g., insects, arachnids, crustaceans), a nematode, an annelid, a helminth, or a mollusc. In some embodiments, the eukaryotic cell is a cell of a multicellular plant, such as an angiosperm plant (which can be a dicot or a monocot),a gymnosperm plant (e.g., a conifer, a cycad, a gnetophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest.

Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L. In some embodiments, a bioreactor may produce at least 1 g of circular RNA. In some embodiments, a bioreactor may produce 1-200 g of circular RNA (e.g., 1-10 g, 1-20 g, 1-50 g, 10-50 g, 10-100 g, 50-100 g, or 50-200 g of circRNA). In some embodiments, the amount produced is measured per liter (e.g., 1-200 g per liter), per batch or reaction (e.g., 1-200 g per batch or reaction), or per unit time (e.g., 1-200 g per hour or per day). In some embodiments, more than one bioreactor may be utilized in series to increase the production capacity (e.g., one, two, three, four, five, six, seven, eight, or nine bioreactors may be used in series).

Methods of making the circRNAs described herein are described in, for example, Khudyakov & Fields, Artificial DNA: Methods and Applications, CRC Press (2002); in Zhao, Synthetic Biology: Tools and Applications, (First Edition), Academic Press (2013); Muller and Appel, from RNA Biol, 2017, 14 (8): 1018-1027; and Egli & Herdewijn, Chemistry and Biology of Artificial Nucleic Acids, (First Edition), Wiley-VCH (2012). Other methods of making circular polyribonucleotides are described, for example, in International Publication No. WO2022/247943, US Patent No. US 11000547, International Publication No. WO2018/191722, International Publication No. WO2019/236673, International Publication No. WO2020/023595, International Publication No. WO2022/204460, International Publication No. WO2022/204464, and International Publication No. WO2022/204466.

Various methods of synthesizing circRNAs are also described elsewhere (see, e.g., US Patent No. US 6210931, US Patent No. US 5773244, US Patent No. US 5766903, US Patent No. US 5712128, US Patent No. US 5426180, US Publication No. US20100137407, International Publication No. WO1992001813, International Publication No. WO2010084371, and Petkovic et al., Nucleic Acids Res.

43:2454-65 (2015); the contents of each of which are herein incorporated by reference in their entirety).

### Circular Polyribonucleotides

The disclosure features circRNA compositions, and methods of making and purifying circRNAs. In some embodiments, a circRNA is produced from a linRNA (e.g., by methods known in the art including by enzymatic ligation or by auto-catalytic RNA). In some embodiments, a linRNA is transcribed from a deoxyribonucleotide template (e.g., a vector, a linearized vector, or a cDNA).

CircRNAs may include features such as one or more coding sequences, one or more non-coding sequences, or a combination thereof. CircRNAs may include one or more coding sequences, for example, a coding sequence that codes for the expression of a polypeptide. Each coding sequence may be operably linked to an internal ribosomal entry site (IRES) or one or more regulatory sequences, or a combination thereof. CircRNAs may include one or more non-coding sequences, for example, a non-coding sequence that binds specifically to a target, such as a protein or a nucleic acid. Features of circRNAs are described, for example, in International Patent Publications WO 2019/118919, WO 2020/023655, WO 2020/180751, WO 2020/180752, WO 2020/181013, WO 2020/198403, WO 2020/257730, WO 2020/257727, WO 2020/252436, each of which is incorporated by reference with respect to the circRNAs described therein.

The size of the circRNA to be purified may be of any size suitable for the purification methods disclosed herein.

For example, the circRNA to be purified may have a length of at least 20,000 nucleotides, at least 19,000 nucleotides, at least 18,000 nucleotides, at least 17,000 nucleotides, at least 16,000 nucleotides, at least 15,000 nucleotides, at least 14,000 nucleotides, at least 13,000 nucleotides, at least 12,000 nucleotides, at least 11,000 nucleotides, at least 10,000 nucleotides, at least 9,000 nucleotides, at least 8,000 nucleotides, at least 7,000 nucleotides, at least 6,000 nucleotides, at least 5,000 nucleotides, at least 4,000 nucleotides, at least 3,000 nucleotides, at least 2,000 nucleotides, at least 1,000 nucleotides, at least 900 nucleotides, at least 800 nucleotides, at least 700 nucleotides, at least 600 nucleotides, at least 500 nucleotides, at least 400 nucleotides, at least 300 nucleotides, at least 200 nucleotides, or at least 100 nucleotides.

For example, the circRNA to be purified may have a length of less than about 20,000 nucleotides, less than about 19,000 nucleotides, less than about 18,000 nucleotides, less than about 17,000 nucleotides, less than about 16,000 nucleotides, less than about 15,000 nucleotides, less than about 14,000 nucleotides, less than about 15,000 nucleotides, less than about 14,0000 nucleotides, less than about 13,000 nucleotide, less than about 12,000 nucleotides, less than about 11,000 nucleotides, less than about 10,000 nucleotides, less than about 9,000 nucleotides, less than about 8,000 nucleotides, less than about 7,000 nucleotides, less than about 6,000 nucleotides, less than about 5,000 nucleotides, less than about 4,000 nucleotides, less than about 3,000 nucleotides, less than about 2,000 nucleotides, less than about 1,000 nucleotides, less than about 900 nucleotides, less than about 800 nucleotides, less than about 700 nucleotides, less than about 600 nucleotides, less than about 500 nucleotides, less than about 400 nucleotides, less than about 300 nucleotides, less than about 200 nucleotides, or less than about 100 nucleotides.

For example, the circRNA to be purified may have a length from about 100 nucleotides to about 20,000 nucleotides (e.g., about 100 nucleotides to about 500 nucleotides, about 100 nucleotides to about 750 nucleotides, about 100 nucleotides to about 1,000 nucleotides, about 100 nucleotides to about 2,500 nucleotides, about 100 nucleotides to about 5,000 nucleotides, about 100 nucleotides to about 10,000 nucleotides, about 500 nucleotides to about 750 nucleotides, about 750 nucleotides to about 1,000 nucleotides, about 750 nucleotides to about 1,250 nucleotides, about 1,000 nucleotides to about 1,250 nucleotides, about 1,000 nucleotides to about 2,000 nucleotides, about 1,000 nucleotides to about 5,000 nucleotides, about 2,500 nucleotides to about 5,000 nucleotides, about 5,000 nucleotides to about 10,000 nucleotides, about 5,000 nucleotides to about 15,000 nucleotides, about 10,000 nucleotides to about 15,000 nucleotides, about 15,000 nucleotides to about 20,000 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1,000 nucleotides, about 1,250 nucleotides, about 1,500 nucleotides, about 1,750 nucleotides, about 2,000 nucleotides, about 3,000 nucleotides, about 4,000 nucleotides, about 5,000 nucleotides, about 6,000 nucleotides, about 7,000 nucleotides, about 8,000 nucleotides, about 9,000 nucleotides, about 10,000 nucleotides, about 11,000 nucleotides, about 12,000 nucleotides, about 13,000 nucleotides, about 14,000 nucleotides, about 15,000 nucleotides, about 16,000 nucleotides, about 17,000 nucleotides, about 18,000 nucleotides, about 19,000 nucleotides, or about 20,000 nucleotides).

Purification of circRNAs from a mixed population of polyribonucleotides according to the methods disclosed herein may be performed on any kind of circRNA, including single- or double-stranded circRNA, circRNA containing secondary structures and circRNAs lacking any secondary structure, labeled or unlabeled circRNA (e.g., fluorescently labeled, radiolabeled, antibody-labeled, among others).

CircRNAs selected for purification may be included of naturally occurring DNA or RNA nucleosides (e.g., adenine, thymidine, guanosine, cytidine, uridine, or inosine), or the circRNA may be included of non-naturally occurring (i.e., modified) nucleobases, internucleoside linkages, or sugars. In some embodiments, the circRNAs selected for purification are included of naturally occurring nucleosides. In cases where the circRNA is modified, it may contain modified ribonucleosides (e.g., containing modified nucleobases or modified ribose moieties) or modified internucleoside linkages (e.g., phosphorothioate and phosphoroamidate, among others). Generally, such modifications are incorporated to stabilize the RNA molecule and to reduce hydrolysis by nucleases. Modifications to circRNA specifically contemplated by the present disclosure include nucleobase modifications described in WO 2020/198403.

In some embodiments, substantially all of the nucleosides or internucleosidic linkages of a circRNA of the disclosure are modified nucleosides. In some embodiments, all of the nucleosides or internucleosidic linkages of the disclosed circRNA are modified nucleosides. CircRNAs in which “substantially all of the nucleosides are modified nucleosides” are largely but not wholly modified and can include not more than five, four, three, two, or one naturally occurring nucleosides. In some embodiments, circRNAs can include not more than five, four, three, two, or one alternative nucleosides.

In some embodiments, a modified circRNA includes at least 1 (e.g., at least 2, 5, 10, 15, 20, 30, 40, 50, 60, 70, 80, 90,100,150, 200, 250, 300, 350, 400, 450, 500, 600, 700, 800, 900, 1000, 1500, 2000, 2500, 3000, 3500, 4000, 4500, 5000, 5500, 6000, 6500, 7000 or more) modified nucleoside. The aforementioned modifications, including modifications to the nucleobase, sugar moiety, or internucleoside linkage, may be incorporated into a circRNA described herein to the extent that they retain the information encoded in the unmodified RNA sequence (e.g., amino acids encoded by each codon) and do not interfere with protein translation.

### Polypeptide Expression Sequences

In some embodiments, the polyribonucleotide described herein includes one or more expression sequences, wherein each expression sequence encodes a polypeptide. In some embodiments, the circular polyribonucleotide includes two, three, four, five, six, seven, eight, nine, ten or more expression sequences.

Each encoded polypeptide may be linear or branched. The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Polypeptides included herein may include naturally occurring polypeptides or non-naturally occurring polypeptides. In some instances, the polypeptide is or includes a functional fragment or variant of a reference polypeptide (e.g., an enzymatically active fragment or variant of an enzyme). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, or a polypeptide for agricultural applications.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, or a thrombolytic.

A polypeptide for agricultural applications may be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide or nematocidal polypeptide), an antigen binding polypeptide (e.g., antigen binding antibody or antibody-like fragments, such as single chain antibodies, nanobodies or other Ig heavy chain or light chain containing polypeptides), an enzyme (e.g., nuclease, amylase, cellulase, peptidase, lipase, chitinase), a peptide pheromone, or a transcription factor.

In some cases, the polyribonucleotide expresses a human protein. In some cases, the polyribonucleotide expresses a non-human protein.

In some embodiments, the polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In some embodiments, the expression sequence includes a polyA sequence (e.g., at the 3′ end of an expression sequence). In some embodiments, the length of a polyA sequence is greater than 10 nucleotides in length. In one embodiment, the polyA sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides). In some embodiments, the polyA sequence is designed according to the descriptions of the polyA sequence in [0202]-[0204] of International Patent Publication No. WO2019/118919A1, which is incorporated herein by reference in its entirety. In some embodiments, the expression sequence lacks a polyA sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

### Internal Ribosomal Entry Site

In some embodiments, the polyribonucleotide described herein includes one or more internal ribosome entry site (IRES) elements. In some embodiments, the IRES is operably linked to one or more expression sequences (e.g., each IRES is operably linked to one or more expression sequences). In embodiments, the IRES is located between a heterologous promoter and the 5′ end of a coding sequence.

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, or a Drosophila. Such viral DNA may be derived from, but is not limited to, picornavirus complementary DNA (cDNA), with EMCV cDNA and poliovirus cDNA. In one embodiment, Drosophila DNA from which an IRES element is derived includes, but is not limited to, an Antennapedia gene from Drosophila melanogaster.

In some embodiments, if present, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, human poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human elF4G, Mouse NDST4L, Human LEF1, Mouse HIF1 alpha, Human n.myc, Mouse Gtx, Human p27kipl, Human PDGF2/c-sis, Human p53, Human Pim-I, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-I, Simian picomavirus, Turnip crinkle virus, Aichivirus, Crohivirus, Echovirus 11, an aptamer to elF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus (EMCV). In a further embodiment, the IRES is an IRES sequence of Theiler's encephalomyelitis virus.

In some embodiments, the IRES sequence has a modified sequence in comparison to the wildtype IRES sequence. In some embodiments, when the last nucleotide of the wild-type IRES is not a cytosine nucleic acid residue, the last nucleotide of the wild-type IRES sequence may be modified such that it is a cytosine residue. For example, in some embodiments, the IRES sequence is a CVB3 IRES sequence wherein the terminal adenosine residue is modified to a cytosine residue. In some embodiments, the IRES sequence is an Enterovirus 71 (EV17) IRES, wherein the terminal guanosine residue is modified to a cytosine residue. In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s).

In some embodiments, a polyribonucleotide described herein includes an IRES (e.g., an IRES operably linked to a coding region). For example, the polyribonucleotide may include any IRES as described in Chen et al. Mol. Cell 81 (20): 4300-4318, 2021; Jopling et al. Oncogene 20:2664-2670, 2001; Baranick et al. PNAS 105 (12): 4733-4738, 2008; Lang et al. Molecular Biology of the Cell 13 (5): 1792-1801, 2002; Dorokhov et al. PNAS 99 (8): 5301-5306, 2002; Wang et al. Nucleic Acids Research 33 (7): 2248-2258, 2005; Petz et a. Nucleic Acids Research 35 (8): 2473-2482, 2007; Chen et al. Science 268:415-417, 1995; Fan et al. Nature Communication 13 (1): 3751-3765, 2022, and International Publication No. WO2021/263124, each of which is hereby incorporated by reference in their entirety.

### Regulatory Elements

In some embodiments, the polyribonucleotide described herein includes one or more regulatory elements. In some embodiments, the polyribonucleotide includes a regulatory element, e.g., a sequence that modifies expression of an expression sequence within the polyribonucleotide.

A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase the amount or number of products expressed for multiple expression sequences attached in tandem. Hence, one regulatory element can enhance the expression of one or more expression sequences. Multiple regulatory elements can also be used, for example, to differentially regulate expression of different expression sequences.

In some embodiments, the regulatory element is a translation modulator. A translation modulator can modulate translation of the expression sequence in the polyribonucleotide. A translation modulator can be a translation enhancer or suppressor. In some embodiments, the polyribonucleotide includes at least one translation modulator adjacent to at least one expression sequence. In some embodiments, the polyribonucleotide includes a translation modulator adjacent each expression sequence. In some embodiments, the translation modulator is present on one or both sides of each expression sequence, leading to separation of the expression products, e.g., peptide(s) and or polypeptide(s).

In some embodiments, the regulatory element is a microRNA (miRNA) or a miRNA binding site.

Further examples of regulatory elements are described, e.g., in paragraphs [0154]-[0161] of International Patent Publication No. WO2019/118919, which is hereby incorporated by reference in its entirety.

### Translation Initiation Sequences

In some embodiments, the polyribonucleotide described herein includes at least one translation initiation sequence. In some embodiments, the polyribonucleotide includes a translation initiation sequence operably linked to an expression sequence.

In some embodiments, the polyribonucleotide encodes a polypeptide and may include a translation initiation sequence, e.g., a start codon. In some embodiments, the translation initiation sequence includes a Kozak or Shine-Dalgarno sequence. In some embodiments, the polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the polyribonucleotide includes at least one translation initiation sequence adjacent to an expression sequence. In some embodiments, the translation initiation sequence provides conformational flexibility to the polyribonucleotide. In some embodiments, the translation initiation sequence is within a substantially single stranded region of the polyribonucleotide. Further examples of translation initiation sequences are described in paragraphs [163]-[0165] of International Patent Publication No. WO2019/118919, which is hereby incorporated by reference in its entirety.

The polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the polyribonucleotide may initiate at a codon which is not the first start codon, e.g., AUG. Translation of the polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC, CAG, CTG.

### Termination Elements

In some embodiments, the polyribonucleotide described herein includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or-1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for a first expression sequence is the same as or continuous with or overlapping with another UTR for a second expression sequence.

Exemplary untranslated regions are described in paragraphs[0197]-[201] of International Patent Publication No. WO2019/118919, which is hereby incorporated by reference in its entirety.

In some embodiments, a circular polyribonucleotide includes a polyA sequence. Exemplary polyA sequences are described in paragraphs [0202]-[0205] of International Patent Publication No. WO2019/118919, which is hereby incorporated by reference in its entirety. In some embodiments, a circular polyribonucleotide lacks a polyA sequence.

In some embodiments, a circular polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a polyA sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′-UTR. In some embodiments, the circular polyribonucleotide lacks a 3′-UTR. In some embodiments, the circular polyribonucleotide lacks a polyA sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Stagger Elements

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide includes one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Non-Coding Sequences In some embodiments, the polyribonucleotide described herein includes one or more non-coding sequence, e.g., a sequence that does not encode the expression of polypeptide. In some embodiments, the polyribonucleotide includes two, three, four, five, six, seven, eight, nine, ten or more non-coding sequences. In some embodiments, the polyribonucleotide does not encode a polypeptide expression sequence.

Noncoding sequences can be natural or synthetic sequences. In some embodiments, a noncoding sequence can alter cellular behavior, such as e.g., lymphocyte behavior. In some embodiments, the noncoding sequences are antisense to cellular RNA sequences.

In some embodiments, the polyribonucleotide includes regulatory nucleic acids that are RNA or RNA-like structures typically from about 5-500 base pairs (bp) (depending on the specific RNA structure, e.g., miRNA 5-30 bps, lncRNA 200-500 bps) and may have a nucleobase sequence identical (complementary) or nearly identical (substantially complementary) to a coding sequence in an expressed target gene within the cell. In embodiments, the circular polyribonucleotide includes regulatory nucleic acids that encode an RNA precursor that can be processed to a smaller RNA, e.g., a miRNA precursor, which can be from about 50 to about 1000 bp, that can be processed to a smaller miRNA intermediate or a mature miRNA.

Long non-coding RNAs (lncRNA) are defined as non-protein coding transcripts longer than 100 nucleotides. Many lncRNAs are characterized as tissue specific. Divergent lncRNAs that are transcribed in the opposite direction to nearby protein-coding genes (include a significant proportion (e.g., about 20%) of total lncRNAs in mammalian genomes) and possibly regulate the transcription of the nearby gene. In one embodiment, the polyribonucleotide provided herein includes a sense strand of a lncRNA. In one embodiment, the polyribonucleotide provided herein includes an antisense strand of a lncRNA.

### Protein-Binding Sequences

In some embodiments, a circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, a circular polyribonucleotide includes at least one immunoprotein binding site, for example to evade immune responses, e.g., cytotoxic T lymphocyte (CTL) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present disclosure, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences including a ribosome binding site, e.g., an initiation codon.

Natural 5′ UTRs bear features which play roles in translation initiation. They harbor signatures like Kozak sequences which are commonly known to be involved in the process by which the ribosome initiates translation of many genes. Kozak sequences have the consensus CCR (A/G) CCAUGG, where R is a purine (adenine or guanine) three bases upstream of the start codon (AUG), which is followed by another ‘G’. 5′ UTR also have been known to form secondary structures which are involved in elongation factor binding.

In some embodiments, a circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, or any other protein that binds RNA.

### Methods of Use

In some embodiments, the polyribonucleotides (e.g., circular polyribonucleotides) made as described herein are used as effectors in therapy or agriculture.

For example, a polyribonucleotide purified by the methods described herein may be administered to a subject (e.g., in a pharmaceutical, veterinary, or agricultural composition). In some embodiments, the subject is a vertebrate animal (e.g., mammal, bird, fish, reptile, or amphibian). In some embodiments, the subject is a human. In some embodiments, the subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

In some embodiments, the disclosure provides a method of modifying a subject by providing to the subject a composition or formulation described herein. In some embodiments, the composition or formulation is or includes a nucleic acid molecule (e.g., a DNA molecule or an RNA molecule described herein), and the polynucleotide is provided to a eukaryotic subject. In some embodiments, the composition or formulation is or includes or a eukaryotic or prokaryotic cell including a nucleic acid described herein.

In some embodiments, the disclosure provides a method of treating a condition in a subject in need thereof by providing to the subject a composition or formulation described herein. In some embodiments, the composition or formulation is or includes a nucleic acid molecule (e.g., a DNA molecule or a polyribonucleotide described herein), and the polynucleotide is provided to a eukaryotic subject. In some embodiments, the composition or formulation is or includes a eukaryotic or prokaryotic cell including a nucleic acid described herein.

In some embodiments, the disclosure provides a method of providing a polyribonucleotide (e.g., circular polyribonucleotide) to a subject by providing a eukaryotic or prokaryotic cell include a polynucleotide described herein to the subject.

### Formulations

In some embodiments of the present disclosure a polyribonucleotide (e.g., a circular polyribonucleotide) or a preparation thereof prepared by the methods described herein may be formulated in composition, e.g., a composition for delivery to a cell, a plant, an invertebrate animal, a non-human vertebrate animal, or a human subject, e.g., an agricultural, veterinary, or pharmaceutical composition. In some embodiments, the polyribonucleotide is formulated in a pharmaceutical composition. In some embodiments, a composition includes a polyribonucleotide and a diluent, a carrier, an adjuvant, or a combination thereof. In a particular embodiment, a composition includes a polyribonucleotide described herein and a carrier or a diluent free of any carrier. In some embodiments, a composition including a polyribonucleotide with a diluent free of any carrier is used for naked delivery of the polyribonucleotide (e.g., circular polyribonucleotide) to a subject.

Pharmaceutical compositions may optionally include one or more additional active substances, e.g., therapeutically and/or prophylactically active substances. Pharmaceutical compositions may optionally include an inactive substance that serves as a vehicle or medium for the compositions described herein (e.g., compositions including circular polyribonucleotides, such as any one of the inactive ingredients approved by the United States Food and Drug Administration (FDA) and listed in the Inactive Ingredient Database). Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference). Non-limiting examples of an inactive substance include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the reference criterion for the amount of linear polyribonucleotide molecules present in the preparation is the presence of no more than 1 ng/ml, 5 ng/ml, 10 ng/ml, 15 ng/ml, 20 ng/mL, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 g/ml, 10 μg/mL, 50 μg/mL, 100 μg/mL, 200 g/mL, 300 μg/mL, 400 μg/mL, 500 μg/mL, 600 μg/mL, 700 μg/mL, 800 μg/mL, 900 μg/mL, 1 mg/mL, 1.5 mg/mL, or 2 mg/ml of linear polyribonucleotide molecules.

In some embodiments, the reference criterion for the amount of circular polyribonucleotide molecules present in the preparation is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the pharmaceutical preparation.

In some embodiments, the reference criterion for the amount of linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.

In some embodiments, the reference criterion for the amount of nicked polyribonucleotide molecules present in the preparation is no more than 0.5% (w/w), 1% (w/w), 2% (w/w), 5% (w/w), 10% (w/w), or 15% (w/w) nicked polyribonucleotide molecules of the total ribonucleotide molecules in the pharmaceutical preparation.

In some embodiments, the reference criterion for the amount of combined nicked and linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the pharmaceutical preparation. In some embodiments, a pharmaceutical preparation is an intermediate pharmaceutical preparation of a final circular polyribonucleotide drug product. In some embodiments, a pharmaceutical preparation is a drug substance or active pharmaceutical ingredient (API). In some embodiments, a pharmaceutical preparation is a drug product for administration to a subject.

In some embodiments, a preparation of circular polyribonucleotides is (before, during or after the reduction of linear RNA) further processed to substantially remove DNA, protein contamination (e.g., cell protein such as a host cell protein or protein process impurities), endotoxin, mononucleotide molecules, and/or a process-related impurity.

### Salts

In some cases, a composition or pharmaceutical composition provided herein includes one or more salts. For controlling the tonicity, a physiological salt such as sodium salt can be included in a composition provided herein. Other salts can include potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can include those of the inorganic ions, such as, for example, sodium, potassium, calcium, magnesium ions, or the like. Such salts can include salts with inorganic or organic acids, such as hydrochloric acid, hydrobromic acid, phosphoric acid, nitric acid, sulfuric acid, methane sulfonic acid, p-toluene sulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can include one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Diluents

In some embodiments, a composition of the disclosure includes a polyribonucleotide, or a preparation thereof prepared by the methods described herein and a diluent.

A diluent can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, or mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the polyribonucleotide (e.g., circular polyribonucleotide) may be delivered as a naked delivery formulation, such as including a diluent. A naked delivery formulation delivers a polyribonucleotide to a cell without the aid of a carrier and without modification or partial or complete encapsulation of the polyribonucleotide, capped polyribonucleotide, or complex thereof.

A naked delivery formulation is a formulation that is free from a carrier and wherein the polyribonucleotide (e.g., circular polyribonucleotide) is without a covalent modification that binds a moiety that aids in delivery to a cell or without partial or complete encapsulation of the polyribonucleotide. In some embodiments, a polyribonucleotide without a covalent modification that binds a moiety that aids in delivery to a cell is a polyribonucleotide that is not covalently bound to a protein, small molecule, a particle, a polymer, or a biopolymer. A polyribonucleotide without covalent modification that binds a moiety that aids in delivery to a cell does not contain a modified phosphate group. For example, a polyribonucleotide without a covalent modification that binds a moiety that aids in delivery to a cell does not contain phosphorothioate, phosphoroselenates, boranophosphates, organophosphate esters, hydrogen phosphonates, phosphoramidates, phosphorodiamidates, alkyl or aryl phosphonates, or phosphotriesters.

In some embodiments, a naked delivery formulation is free of any or all of: transfection reagents, cationic carriers, carbohydrate carriers, nanoparticle carriers, or protein carriers. In some embodiments, a naked delivery formulation is free from phytoglycogen octenyl succinate, phytoglycogen beta-dextrin, anhydride-modified phytoglycogen beta-dextrin, lipofectamine, polyethyleneimine, poly(trimethylenimine), poly (tetramethylenimine), polypropylenimine, aminoglycoside-polyamine, dideoxy-diamino-b-cyclodextrin, spermine, spermidine, poly(2-dimethylamino) ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy) propyl]-N,N,N-trimethylammonium chloride (DOTMA), I-[2-(oleoyloxy) ethyl]-2-oleyl-3-(2-hydroxyethyl) imidazolinium chloride (DOTIM), 2,3-dioleyloxy-N-[2 (sperminecarboxamido) ethyl]-N, N-dimethyl-I-propanaminium trifluoroacetate (DOSPA), 3B-[N—(N\N′-Dimethylaminoethane)-carbamoyl] Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N, N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, or 2-propanol. Examples of a buffer include 2-(N-morpholino) ethane sulfonic acid (MES), Bis-Tris, 2-[(2-amino-2-oxoethyl)-(carboxymethyl) amino] acetic acid (ADA), N-(2-Acetamido)-2-aminoethanesulfonic acid (ACES), piperazine-N,N′-bis(2-ethanesulfonic acid) (PIPES), 2-[1,3-dihydroxy-2-(hydroxymethyl) propan-2-yl]amino] ethanesulfonic acid (TES), 3-(N-morpholino) propane sulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di (tetradecanoyloxy) propyl-I-0-(w-methoxy (polyethoxy) ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), or combinations of the foregoing. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, and WO2017/099823, the contents of all of which are incorporated herein by reference in their entirety.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or Ill of US2015/0203446; I or Ill of US2015/0005363; I, IA, IB, IC, ID, II, IIA, IIB, IIC, IID, or III-XXIV of US2014/0308304; of US2013/0338210; I, II, III, or IV of WO2009/132131; A of

US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; 1-5 or 1-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-I-trans PE, I-stearoyl-2-oleoyl-phosphatidylethanolamine (SOPE), hydrogenated soy phosphatidylcholine (HSPC), egg phosphatidylcholine (EPC), dioleoyl phosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacyl phosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, non phosphorus lipids such as, e.g., stearyl amine, dodeeylamine, hexadecylamine, acetyl palmitate, glycerol ricin oleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, or the like. Other non-cationic lipids are described in WO2017/099823 or US patent publication US2018/0028664, the contents of which is incorporated herein by reference in their entirety.

### Numbered Embodiments

[1] A method for producing an enriched population of circular polyribonucleotides (circRNA), the method including: (a) providing a sample including a population of polyribonucleotides including circRNA and linear polyribonucleotides (linRNA); and (b) separating the circRNA from the linRNA under denaturing conditions that do not include the use of gel electrophoresis, thereby producing an enriched population of circRNA.

[2] A method for producing an enriched population of circRNA, the method including: (a) providing a sample including a population of polyribonucleotides including circRNA and linRNA; and (b) exposing the population of polyribonucleotides to denaturing conditions, thereby enriching the population of circRNA.

[3] The method of embodiment [1] or [2], wherein: (i) the total weight of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 200 ng/μL.

[4] A method for producing an enriched population of circRNA, the method including: (a) providing a sample including a population of polyribonucleotides including circRNA and linRNA, wherein: (i) the total weight of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 200 ng/μL; and (b) separating the circRNA from the linRNA under denaturing conditions, thereby producing an enriched population of circRNA.

[5] The method of any one of embodiments [1]-[4], wherein the total weight of polyribonucleotides in the population of polyribonucleotides is between 1 μg and 1000 mg.

[6] The method of any one of embodiments [1]-[5], wherein the total volume of the sample including the population of polyribonucleotides is between 500 μl and 1000 mL.

[7] The method any one of embodiments [1]-[6], wherein the concentration of the population of polyribonucleotides in the sample is between 200 ng/μL and 50 mg/mL.

[8] The method of any one of embodiments [4]-[7], wherein the separating step (b) is performed under denaturing conditions that do not include the use of gel electrophoresis.

[9] The method of any one of embodiments [1]-[8], wherein the enriched population of circRNA is substantially free of one or more impurities or by-products.

[10] The method of embodiment [9], wherein the one or more impurities or by-products include polyacrylamide, boric acid, magnesium, or ethylenediaminetetraacetic acid (EDTA).

[11] The method of any one of embodiments [1]-[10], wherein the denaturing conditions include a temperature of at least 50° C.

[12] The method of embodiment [11], wherein the denaturing conditions include a temperature of between 50° C. and 85° C.

[13] The method of any one of embodiments [1]-[12], wherein the denaturing conditions include a temperature of at least 50° C. followed by a temperature of not greater than 8° C. within a time period of no greater than 30 seconds.

[14] The method of any one of embodiments [1]-[13], wherein the denaturing conditions include a pH of less than 5 or greater than 9. [15] The method of embodiment [14], wherein the denaturing conditions include a pH of less than 5. [16] The method of embodiment [14], wherein the denaturing conditions include a pH of greater than 9. [17] The method of any one of embodiments [1]-[16], wherein the denaturing conditions include a chemical treatment.

[18] The method of embodiment [17], wherein the chemical treatment includes treatment with an acid, base, organic solvent, chaotropic agent, crowding agent, chelator, detergent, or salt solution.

[19] The method of embodiment [18], wherein the acid includes between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[20] The method of embodiment [18] or [19], wherein the base includes between 1 mM and 500 mM sodium hydroxide, potassium hydroxide, imidazole, histidine, sodium bicarbonate, guanidine, or triethylamine.

[21] The method of any one of embodiments [18]-[20], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[22] The method of any one of embodiments [18]-[21], wherein the chaotropic agent includes between 100 mM and 8 M urea, guanidinium chloride, lithium perchlorate, or polyethylene glycol (PEG). [23] The method of any one of embodiments [18]-[22], wherein the chaotropic agent includes between 100 mM and 8 M n-dodecyl β-d-maltoside, n-octylglucoside, CHAPS, or deoxycholate.

[24] The method of any one of embodiments [18]-[23], wherein the crowding agent includes between 100 mM and 8 M PEG, or urea.

[25] The method of any one of embodiments [18]-[24], wherein the chelator includes between 1 mM and 10 mM ethylene glycol-bis(p3-aminoethyl ether)-N,N,N′,N′-tetra acetic acid (EGTA) or derivatives thereof, ethylenediaminetetraacetic acid (EDTA) or derivatives thereof, nitrilotriacetic acid (NTA), iminodisuccinic acid (IDS), polyaspartic acid, S,S-ethylenediamine-N,N′-disuccinic acid (EDDS), or methylglycinediacetic acid (MGDA).

[26] The method of any one of embodiments [18]-[25], wherein the detergent includes between 0.005% and 0.05% (v/v) Nonidet P-40 (NP40), CHAPS, octyl β-D-glucopyranoside, n-dodecyl β-d-maltoside, Tween-20, or Tween-80.

[27] A method for producing an enriched population of circular polyribonucleotides (circRNA), the method including: (a) providing a sample including a population of polyribonucleotides including circRNA and linear polyribonucleotides (linRNA); and (b) separating the circRNA from the linRNA at a temperature of at least 50° C., thereby producing an enriched population of circRNA, wherein the separating does not include the use of gel electrophoresis.

[28] A method for producing an enriched population of circRNA, the method including: (a) providing a sample including a population of polyribonucleotides including circRNA and linRNA, wherein: (i) the total mass of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 200 ng/μL; and (b) separating the circRNA from the linRNA at a temperature of at least 50° C., thereby producing an enriched population of circRNA.

[29] A method for producing an enriched population of circRNA, the method including: (a) providing a sample including a population of polyribonucleotides including circRNA and linRNA; and (b) exposing the population of polyribonucleotides to a temperature of at least 50° C., thereby enriching the population of circRNA.

[30] The method of any one of embodiments [27]-[29], wherein the temperature is between 50° C. and 85° C.

[31] A method for producing an enriched population of circRNA, the method including: (a) providing a sample including a population of polyribonucleotides including circRNA and linRNA; and (b) separating the circRNA from the linRNA at a pH of less than 5 or greater than 9, thereby producing an enriched population of circRNA, wherein the separating does not include the use of gel electrophoresis.

[32] A method for producing an enriched population of circRNA, the method including: (a) providing a sample including a population of polyribonucleotides including circRNA and linRNA, wherein: (i) the total mass of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 200 ng/μL; and (b) separating the circRNA from the linRNA at a pH of less than 5 or greater than 9, thereby producing an enriched population of circRNA.

[33] A method for producing an enriched population of circRNA, the method including: (a) providing a sample including a population of polyribonucleotides including circRNA and linRNA; and (b) exposing the population of polyribonucleotides to a pH of less than 5 or greater than 9, thereby enriching the population of circRNA.

[34] The method of any one of embodiments [31]-[33], wherein the pH is less than 5. [35] The method of any one of embodiments [31]-[33], wherein the pH is greater than 9. [36] A method for producing an enriched population of circRNA, the method including: (a) providing a sample including a population of polyribonucleotides including circRNA and linRNA; and (b) separating the circRNA from the linRNA under conditions including an acid, base, organic solvent, chaotropic agent, crowding agent, chelator, detergent, or salt solution, thereby producing an enriched population of circRNA, wherein the separating does not include the use of gel electrophoresis.

[37] A method for producing an enriched population of circRNA, the method including: (a) providing a sample including a population of polyribonucleotides including circRNA and linRNA, wherein: (i) the total mass of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 200 ng/μL; and (b) separating the circRNA from the linRNA under conditions including an acid, base, organic solvent, chaotropic agent, crowding agent, chelator, detergent, or salt solution, thereby producing an enriched population of circRNA.

[38] A method for producing an enriched population of circRNA, the method including: (a) providing a sample including a population of polyribonucleotides including circRNA and linRNA; and (b) exposing the population of polyribonucleotides to an acid, base, organic solvent, chaotropic agent, crowding agent, chelator, detergent, or salt solution, thereby enriching the population of circRNA.

[39] The method of any one of embodiments [36]-[38], wherein the acid includes at least 0.5% (v/v) acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[40] The method of any one of embodiments [36]-[38], wherein the base includes between 1 mM and 500 mM sodium hydroxide, potassium hydroxide, imidazole, histidine, sodium bicarbonate, guanidine, or triethylamine.

[41] The method of any one of embodiments [36]-[38], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[42] The method of any one of embodiments [36]-[38], wherein the chaotropic agent includes between 100 mM and 8 M urea, guanidinium chloride, lithium perchlorate, or polyethylene glycol (PEG).

[43] The method of any one of embodiments [36]-[38], wherein the chaotropic agent includes between 100 mM and 8 M n-dodecyl β-d-maltoside, n-octylglucoside, CHAPS, or deoxycholate.

[44] The method of any one of embodiments [36]-[38], wherein the crowding agent includes between 100 mM and 8 M polyethylene glycol (PEG), or urea.

[45] The method of any one of embodiments [36]-[38], wherein the chelator includes between 1 mM and 10 mM EGTA or derivatives thereof, EDTA or derivatives thereof, NTA, IDS, EDDS, or MGDA.

[46] The method of any one of embodiments [36]-[38], wherein the detergent includes between 0.005% and 0.05% (v/v) NP40, CHAPS, octyl β-D-glucopyranoside, n-dodecyl β-d-maltoside, Tween-20, or Tween-80.

[47] The method of any one of embodiments [1]-[46], wherein step (b) includes performing column chromatography on the population of polyribonucleotides, wherein performing column chromatography includes an equilibration step, sample loading step, column washing step, and elution step.

[48] The method of embodiment [47], wherein the separating is performed during the sample loading step.

[49] The method of embodiment [46] or [47], wherein the separating is performed during the column washing step.

[50] The method of any one of embodiments [47]-[49], wherein the separating is performed during the elution step.

[51] The method of any one of embodiments [47]-[50], wherein the column chromatography includes fast protein liquid chromatography (FPLC), high-pressure liquid chromatography (HPLC), hydrophobic interaction chromatography (HIC), anion exchange chromatography (AEC), mixed mode chromatography, or affinity chromatography.

[52] The method of embodiment [51], wherein the AEC includes use of an anion exchange resin selected from the group consisting of styrene-divinylbenzene, silica, sepharose, cellulose, dextran, epoxy polyamine, methacrylate, agarose, and acrylic.

[53] The method of embodiment [52], wherein the anion exchange resin includes an ion exchanger selected from the group consisting of quaternary ammonium, amino ethyl, diethylaminoethyl, and diethylaminopropyl.

[54] The method of embodiment [52] or [53], wherein the anion exchange resin includes beads, wherein the beads have a bead diameter of 45-165 μm and a pore size of diameter 100-1000 nm.

[55] The method of any one of embodiments [51]-[54], wherein the AEC includes use of a linear gradient elution or a step isocratic elution.

[56] The method of any one of embodiments [51]-[55], wherein the AEC includes use of a flow rate that is between 1 mL/min and 150 mL/min.

[57] The method of embodiment [51], wherein the FPLC is reversed phase-FPLC (RP-FPLC).

[58] The method of any one of embodiments [1]-[57], wherein step (b) is performed by pooling multiple fractions of purified circRNA.

[59] The method of any one of embodiments [1]-[58], wherein the circRNA and the linRNA have the same ribonucleotides sequence.

[60] The method of any one of embodiments [1]-[59], wherein the circRNA and the linRNA have the same mass.

[61] The method of any one of embodiments [1]-[60], wherein the circRNA and the linRNA lack a poly(A) tail.

[62] The method of any one of embodiments [1]-[61], wherein the method includes exonuclease digestion of the linRNA.

[63] The method of any one of embodiments [1]-[61], wherein the method does not include exonuclease digestion of the linRNA.

[64] The method of any one of embodiments [1]-[63], wherein the method does not include a selective modification to the circRNA or to the linRNA that improves enrichment of the circRNA.

[65] The method of any one of embodiments [1]-[64], wherein the percent (w/w) of the circRNA in the enriched population of circRNA is 2-fold greater than the percent (w/w) of the circRNA in the polyribonucleotide population.

[66] The method of any one of embodiments [1]-[65], wherein the percent (w/w) of the circRNA in the enriched population of circRNA is at least 65%.

[67] The method of any one of embodiments [1]-[66], wherein the percent (w/w) of the linRNA in the enriched population of circRNA is less than 35%.

[68] The method of any one of embodiments [1]-[67], wherein the circRNA includes a length of 1,000 nucleotides or less.

[69] A composition including a population of polyribonucleotides including circRNA and linRNA, wherein the population of polyribonucleotides is in solution under denaturing conditions, and wherein: (i) the total mass of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 500 ng/μL.

[70] A composition including a population of polyribonucleotides including circRNA and linRNA, wherein the population of polyribonucleotides is in solution under denaturing conditions, and wherein, the solution is substantially free of the one or more impurities or by-products.

[71] A composition including a population of polyribonucleotides including circRNA and linRNA, wherein:
(a) the composition is obtained from a sample including a population of nucleic acids;
(b) the composition has been exposed to one or more denaturing conditions; and
(c) the composition is substantially free of one or more impurities or by-products.

[72] A composition including an enriched population of circRNA, wherein:
(a) the composition is obtained from a sample including a population of polyribonucleotides including circRNA and linRNA;
(b) the composition has been exposed to one or more denaturing conditions; and
(c) the composition is substantially free of one or more impurities or by-products.

[73] The composition of any one of embodiments [69]-[72], wherein the one or more impurities or by-products include polyacrylamide, boric acid, magnesium, or EDTA.

[74] The composition of any one of embodiments [69]-[73], wherein the denaturing conditions include a temperature of at least 50° C.

[75] The composition of any one of embodiments [69]-[74], wherein the denaturing conditions include a temperature of between 50° C. and 85° C.

[76] The composition of any one of embodiments [69]-[75], wherein the denaturing conditions include a temperature of at least 50° C. followed by a temperature of not greater than 8° C.

[77] The composition of any one of embodiments [69]-[73], wherein the denaturing conditions include a pH of less than 5 or greater than 9.

[78] The composition of any one of embodiments [69]-[73], wherein the denaturing conditions include a chemical treatment.

[79] The composition of embodiment [78], wherein the chemical treatment includes treatment with an acid, base, organic solvent, chaotropic agent, crowding agent, chelator, detergent, or salt solution.

[80] The composition of embodiment [79], wherein the acid includes between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[81] The composition of embodiment [79] or [80], wherein the base includes between 1 mM and 500 mM sodium hydroxide, potassium hydroxide, imidazole, histidine, sodium bicarbonate, guanidine, or triethylamine.

[82] The composition of any one of embodiments [79]-[81], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[83] The composition of any one of embodiments [79]-[82], wherein the chaotropic agent includes between 100 mM and 8 M of urea, guanidinium chloride, lithium perchlorate, or polyethylene glycol (PEG).

[84] The composition of any one of embodiments [79]-[82], wherein the chaotropic agent includes between 100 mM and 8 M of n-dodecyl β-d-maltoside, n-octylglucoside, CHAPS, or deoxycholate.

[85] The method of any one of embodiments [79]-[84], wherein the crowding agent includes between 100 mM and 8 M polyethylene glycol (PEG), or urea.

[86] The composition of any one of embodiments [79]-[85], wherein the chelator includes between 1 mM and 10 mM EGTA or derivatives thereof, EDTA or derivatives thereof, NTA, IDS, EDDS, or MGDA.

[87] The composition of any one of embodiments [79]-[86], wherein the detergent includes between 0.005% and 0.05% (v/v) NP40, CHAPS, octyl β-D-glucopyranoside, n-dodecyl β-d-maltoside, Tween-20, or Tween-80. [88] The composition of any one of embodiments [69]-[87], wherein the percent (w/w) of the circRNA in the enriched population of circRNA is 2-fold greater than the percent (w/w) of the circRNA in the polyribonucleotide population.

[89] The composition of any one of embodiments [69]-[88], wherein the percent (w/w) of the circRNA in the enriched population of circRNA is at least 65%.

[90] The composition of any one of embodiments [69]-[89], wherein the percent (w/w) of the linRNA in the enriched population of circRNA is less than 35%.

[91] The composition of any one of embodiments [69]-[90], wherein the circRNA and the linRNA have the same ribonucleotides sequence.

[92] The composition of any one of embodiments [69]-[91], wherein the circRNA and the linRNA have the same mass.

[93] The composition of any one of embodiments [69]-[92], wherein the circRNA and the linRNA lack a poly(A) tail.

[94] The composition of any one of embodiments [69]-[93], wherein the circRNA includes a length of 1,000 nucleotides or less.

[95] A method of determining the purity of a circRNA, the method including: (a) providing a sample including a population of polyribonucleotides including circRNA and linRNA; (b) separating the circRNA from the linRNA under denaturing conditions by chromatography; and (c) collecting a chromatogram of the sample including a peak for the circRNA and a peak for the linRNA; (d) calculating the area under each peak to determine the purity of the circRNA in the sample.

[96] The method of embodiment [95], wherein the denaturing conditions do not include the use of gel electrophoresis.

[97] The method of embodiment [95] or [96], wherein the denaturing conditions include a temperature of at least 50° C.

[98] The method of embodiment [97], wherein the denaturing conditions include a temperature of between 50° C. and 85° C.

[99] The method of any one of embodiments [95]-[98], wherein the denaturing conditions include a temperature of at least 50° C. followed by a temperature of not greater than 8° C. within a time period of no greater than 30 seconds.

[100] The method of any one of embodiments [95]-[99], wherein the denaturing conditions include a pH of less than 5 or greater than 9.

[101] The method of embodiment [100], wherein the denaturing conditions include a pH of less than 5.

[102] The method of embodiment [100], wherein the denaturing conditions include a pH of greater than 9.

[103] The method of any one of embodiments [95]-[102], wherein the denaturing conditions include a chemical treatment.

[104] The method of embodiment [103], wherein the chemical treatment includes treatment with an acid, base, organic solvent, chaotropic agent, crowding agent, chelator, detergent, or salt solution.

[105] The method of embodiment [104], wherein the acid includes between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[106] The method of embodiment [104] or [105], wherein the base includes between 1 mM and 500 mM sodium hydroxide, potassium hydroxide, imidazole, histidine, sodium bicarbonate, guanidine, or triethylamine.

[107] The method of any one of embodiments [104]-[106], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[108] The method of any one of embodiments [104]-[107], wherein the chaotropic agent includes between 100 mM and 8 M urea, guanidinium chloride, lithium perchlorate, or polyethylene glycol (PEG).

[109] The method of any one of embodiments [104]-[108], wherein the chaotropic agent includes between 100 mM and 8 M n-dodecyl β-d-maltoside, n-octylglucoside, CHAPS, or deoxycholate.

[110] The method of any one of embodiments [104]-[109], wherein the crowding agent includes between 100 mM and 8 M polyethylene glycol (PEG), or urea.

[111] The method of any one of embodiments [104]-[110], wherein the chelator includes between 1 mM and 10 mM EGTA or derivatives thereof, EDTA or derivatives thereof, NTA, IDS, polyaspartic acid, EDDS, or MGDA.

[112] The method of any one of embodiments [104]-[111], wherein the detergent includes between 0.005% and 0.05% (v/v) Nonidet P-40 (NP40), CHAPS, octyl β-D-glucopyranoside, n-dodecyl β-d-maltoside, Tween-20, or Tween-80.

[113] The method of any one of embodiments [95]-[112], wherein the chromatography includes liquid chromatography.

[114] The method of embodiment [113], wherein the liquid chromatography is selected from the group consisting of FPLC, HPLC, HIC, AEC, MMC, or affinity chromatography.

[115] The method of any of embodiments [95]-[114], wherein the relative standard deviation (RSD) of the purity is less than 5%.

[116] The method of any one of embodiments [95]-[115], wherein the circRNA includes a length of less 1,000 nucleotides or less.

[117] A method for producing an enriched population of circRNA, the method including the steps of (a) providing a sample including a population of polyribonucleotides including circRNA and linRNA; and (b) separating the circRNA from the linRNA under denaturing conditions that do not include the use of gel electrophoresis, thereby producing an enriched population of circRNA.

[118] A method for producing an enriched population of circRNA, the method including: (a) providing a sample including a population of polyribonucleotides including circRNA and linRNA; and (b) exposing the population of polyribonucleotides to denaturing conditions, thereby enriching the population of circRNA.

[119] The method of embodiment or [117] [118], wherein: (i) the total weight of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 200 ng/μL.

[120] The method of embodiment or [118] [119], wherein the separating step (b) is performed under denaturing conditions that do not include the use of gel electrophoresis; and/or wherein the enriched population of circRNA is substantially free of one or more impurities or by-products, wherein the one or more impurities or by-products include polyacrylamide, boric acid, magnesium, or ethylenediaminetetraacetic acid (EDTA).

[121] The method of any one of embodiments [117]-[120], wherein the denaturing conditions include a temperature of at least 50° C.; or wherein the denaturing conditions include a temperature of at least 50° C. followed by a temperature of not greater than 8° C. within a time period of no greater than 30 seconds.

[122] The method of any one of embodiments [117]-[121], wherein the denaturing conditions include a pH of less than 5 or greater than 9.

[123] The method of any one of embodiments [117]-[122], wherein the denaturing conditions include a chemical treatment including treatment with an acid, base, organic solvent, chaotropic agent, crowding agent, chelator, detergent, or salt solution.

[124] The method of embodiment [123], wherein the acid includes between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[125] The method of embodiment or [124], wherein the base includes between 1 mM and 500 mM sodium hydroxide, potassium hydroxide, imidazole, histidine, sodium bicarbonate, guanidine, or

[126] The method of any one of embodiments [123]-[125], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[127] The method of any one of embodiments [123]-[126], wherein the chaotropic agent includes between 100 mM and 8 M urea, guanidinium chloride, lithium perchlorate, or polyethylene glycol (PEG); and/or wherein the chaotropic agent includes between 100 mM and 8 M n-dodecyl β-d-maltoside, n-octylglucoside, CHAPS, or deoxycholate.

[128] The method of any one of embodiments [123]-[127], wherein the crowding agent includes between 100 mM and 8 M PEG, or urea.

[129] The method of any one of embodiments [123]-[128], wherein the chelator includes between 1 mM and 10 mM ethylene glycol-bis(β-aminoethyl ether)-N,N,N, N-tetra acetic acid (EGTA) or derivatives thereof, ethylenediaminetetraacetic acid (EDTA) or derivatives thereof, nitrilotriacetic acid (NTA), iminodisuccinic acid (IDS), polyaspartic acid, S,S-ethylenediamine-N,N′-disuccinic acid (EDDS), or methylglycinediacetic acid (MGDA).

[130] The method of any one of embodiments [123]-[129], wherein the detergent includes between 0.005% and 0.05% (v/v) Nonidet P-40 (NP40), CHAPS, octyl β-D-glucopyranoside, n-dodecyl β-d-maltoside, Tween-20, or Tween-80.

[131] The method of any one of embodiments [117]-[130], wherein step (b) includes performing column chromatography on the population of polyribonucleotides, wherein performing column chromatography includes an equilibration step, sample loading step, column washing step, and elution step; wherein the separating is performed during the sample loading step, the column washing step, and/or during the elution step.

[132] The method of embodiment [131], wherein the column chromatography includes fast protein liquid chromatography (FPLC), high-pressure liquid chromatography (HPLC), hydrophobic interaction chromatography (HIC), anion exchange chromatography (AEC), mixed mode chromatography, or affinity chromatography.

[133] The method of embodiment [132], wherein the AEC includes (i) use of an anion exchange resin, wherein the anion exchange resin is selected from the group consisting of styrene-divinylbenzene, silica, sepharose, cellulose, dextran, epoxy polyamine, methacrylate, agarose, and acrylic; and/or wherein the anion exchange resin includes an ion exchanger selected from the group consisting of quaternary ammonium, amino ethyl, diethylaminoethyl, and diethylaminopropyl; and/or wherein the anion exchange resin includes beads, wherein the beads have a bead diameter of 45-165 μm and a pore size of diameter 100-1000 nm; and/or (ii) use of a linear gradient elution or a step isocratic elution; and/or (iii) use of a flow rate that is between 1 mL/min and 150 mL/min.

[134] The method of any one of embodiments [117]-[133], wherein step (b) is performed by pooling multiple fractions of purified circRNA.

[135] The method of any one of embodiments [117]-[134], wherein the circRNA and the linRNA have the same ribonucleotides sequence; and/or wherein the circRNA and the linRNA have the same mass; and/or wherein the circRNA and the linRNA lack a poly(A) tail.

[136] The method of any one of embodiments [117]-[135], wherein the method includes exonuclease digestion of the linRNA or wherein the method does not include exonuclease digestion of the linRNA; and/or wherein the method does not include a selective modification to the circRNA or to the linRNA that improves enrichment of the circRNA.

[137] The method of any one of embodiments [117]-[136], wherein the percent (w/w) of the circRNA in the enriched population of circRNA is 2-fold greater than the percent (w/w) of the circRNA in the polyribonucleotide population; and/or wherein the percent (w/w) of the circRNA in the enriched population of circRNA is at least 65%; and/or wherein the percent (w/w) of the linRNA in the enriched population of circRNA is less than 35%.

[138] The method of any one of embodiments [117]-[137], wherein the circRNA includes a length of 1,000 nucleotides or less.

[139] A composition including a population of polyribonucleotides including circRNA and linRNA, wherein the population of polyribonucleotides is in solution under denaturing conditions, and wherein: (i) the total mass of polyribonucleotides in the population of polyribonucleotides is at least 1 μg; (ii) the total volume of the sample including the population of polyribonucleotides is at least 500 μL; or (iii) the concentration of the population of polyribonucleotides in the sample is at least 500 ng/μL.

[140] A composition including an enriched population of circRNA, wherein: (a) the composition is obtained from a sample including a population of polyribonucleotides including circRNA and linRNA; (b) the composition has been exposed to one or more denaturing conditions; and (c) the composition is substantially free of one or more impurities or by-products.

[141] The composition of embodiment [139] or [140], wherein the one or more impurities or by-products include polyacrylamide, boric acid, magnesium, or EDTA.

[142] The composition of any one of embodiments [139]-[141], wherein the denaturing conditions include a temperature of at least 50° C.; or wherein the denaturing conditions include a temperature of at least 50° C. followed by a temperature of not greater than 8° C.

[143] The composition of any one of embodiments [139]-[142], wherein the denaturing conditions include a pH of less than 5 or greater than 9.

[144] The composition of any one of embodiments [139]-[143], wherein the denaturing conditions include a chemical treatment including treatment with an acid, base, organic solvent, chaotropic agent, crowding agent, chelator, detergent, or salt solution.

[145] The composition of embodiment [144], wherein the acid includes between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[146] The composition of embodiment [144] or [145], wherein the base includes between 1 mM and 500 mM sodium hydroxide, potassium hydroxide, imidazole, histidine, sodium bicarbonate, guanidine, or triethylamine.

[147] The composition of any one of embodiments [144]-[146], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[148] The composition of any one of embodiments [144]-[147], wherein the chaotropic agent includes between 100 mM and 8 M of urea, guanidinium chloride, lithium perchlorate, or polyethylene glycol (PEG); and/or wherein the chaotropic agent includes between 100 mM and 8 M of n-dodecyl β-d-maltoside, n-octylglucoside, CHAPS, or deoxycholate.

[149] The method of any one of embodiments [144]-[148], wherein the crowding agent includes between 100 mM and 8 M polyethylene glycol (PEG), or urea.

[150] The composition of any one of embodiments [144]-[149], wherein the chelator includes between 1 mM and 10 mM EGTA or derivatives thereof, EDTA or derivatives thereof, NTA, IDS, EDDS, or MGDA.

[151] The composition of any one of embodiments [144]-[150], wherein the detergent includes between 0.005% and 0.05% (v/v) NP40, CHAPS, octyl β-D-glucopyranoside, n-dodecyl β-d-maltoside, Tween-20, or Tween-80.

[152] The composition of any one of embodiments [139]-[151], wherein the percent (w/w) of the circRNA in the enriched population of circRNA is 2-fold greater than the percent (w/w) of the circRNA in the polyribonucleotide population; and/or wherein the percent (w/w) of the circRNA in the enriched population of circRNA is at least 65%; and/or wherein the percent (w/w) of the linRNA in the enriched population of circRNA is less than 35%.

[153] The composition of any one of embodiments [139]-[152], wherein the circRNA and the linRNA have the same ribonucleotides sequence; and/or wherein the circRNA and the linRNA have the same mass; and/or wherein the circRNA and the linRNA lack a poly(A) tail.

[154] The composition of any one of embodiments [139]-[153], wherein the circRNA includes a length of 1,000 nucleotides or less.

[155] A method of determining the purity of a circRNA, the method including: (a) providing a sample including a population of polyribonucleotides including circRNA and linRNA; (b) separating the circRNA from the linRNA under denaturing conditions by chromatography; and (c) collecting a chromatogram of the sample including a peak for the circRNA and a peak for the linRNA; (d) calculating the area under each peak to determine the purity of the circRNA in the sample.

[156] The method of embodiment [155], wherein the denaturing conditions do not include the use of gel electrophoresis.

[157] The method of embodiment or [156], wherein the denaturing conditions include a temperature of at least 50° C.; or wherein the denaturing conditions include a temperature of at least 50° C. followed by a temperature of not greater than 8° C. within a time period of no greater than 30 seconds.

[158] The method of any one of embodiments [155]-[157], wherein the denaturing conditions include a pH of less than 5 or greater than 9.

[159] The method of any one of embodiments [155]-[158], wherein the denaturing conditions include a chemical treatment including treatment with an acid, base, organic solvent, chaotropic agent, crowding agent, chelator, detergent, or salt solution.

[160] The method of embodiment [159], wherein the acid includes between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

[161] The method of embodiment or [160], wherein the base includes between 1 mM and 500 mM sodium hydroxide, potassium hydroxide, imidazole, histidine, sodium bicarbonate, guanidine, or

[162] The method of any one of embodiments [159]-[161], wherein the organic solvent includes at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

[163] The method of any one of embodiments [159]-[162], wherein the chaotropic agent includes between 100 mM and 8 M urea, guanidinium chloride, lithium perchlorate, or polyethylene glycol (PEG); and/or wherein the chaotropic agent includes between 100 mM and 8 M n-dodecyl β-d-maltoside, n-octylglucoside, CHAPS, or deoxycholate.

[164] The method of any one of embodiments [159]-[162], wherein the crowding agent includes between 100 mM and 8 M polyethylene glycol (PEG), or urea.

[165] The method of any one of embodiments [159]-[164], wherein the chelator includes between 1 mM and 10 mM EGTA or derivatives thereof, EDTA or derivatives thereof, NTA, IDS, polyaspartic acid, EDDS, or MGDA.

[166] The method of any one of embodiments [159]-[165], wherein the detergent includes between 0.005% and 0.05% (v/v) Nonidet P-40 (NP40), CHAPS, octyl β-D-glucopyranoside, n-dodecyl β-d-maltoside, Tween-20, or Tween-80.

[167] The method of any one of embodiments [155]-[166], wherein the chromatography is liquid chromatography, wherein the liquid chromatography is selected from the group consisting of FPLC, HPLC, HIC, AEC, MMC, or affinity chromatography.

[168] The method of any of embodiments [155]-[167], wherein the relative standard deviation (RSD) of the purity is less than 5%.

[169] The method of any one of embodiments [155]-[168], wherein the circRNA includes a length of 1,000 nucleotides or less.

### Examples

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1: Enrichment of CircRNA Under Denaturing Conditions from a Sample Containing a Mixed Population of RNA that Includes CircRNA and LinRNA

According to the presently disclosed methods, a person of ordinary skill in the art can produce an enriched population of circular RNA (circRNA) from a mixed population of polyribonucleotides containing circRNA, linear RNA (linRNA), or other impurities or by-products (e.g., impurities or by-products described herein) by subjecting the sample to one or more denaturing conditions described herein.

First, a sample containing a mixed population of polyribonucleotides containing circRNA and linRNA is obtained. The sample may contain a total mass of polyribonucleotides that is at least 200 μg, have a total volume of at least 500 μL, or have a concentration of polyribonucleotides in the sample that is at least 200 ng/μL. The in vitro transcription (IVT) RNA sample is subsequently prepared and subjected to initial purification steps well-known in the art. The IVT sample may be pre-processed prior to purification to remove large contaminants, debris, and other macromolecules. The IVT sample may also be centrifuged to separate out the precipitation and supernatant. Several rounds of buffer washes and centrifugation may be performed, as needed. Purification is performed by subjecting the IVT sample to one or more denaturing conditions described herein (e.g., thermal denaturation, pH denaturation, chemical denaturation with an acid, base, organic solvent, chaotropic agent, crowding agent, chelator, detergent, or salt solution), wherein the denaturing conditions substantially denature structural implications within the linRNA and circRNA. Purification by denaturing conditions is performed alone or in combination with column chromatographic separation techniques known in the art (e.g., anion exchange chromatography, hydrophobic interaction chromatography, mixed mode chromatography, or affinity chromatography). In the case that column chromatographic purification is performed, exposure of the sample containing the polyribonucleotides to denaturing conditions is performed during the equilibration step, sample loading step, column washing step, or elution step. The method may or may not include exonuclease-mediated digestion of the linRNA molecules in the sample.

Accordingly, the purification is performed to the extent that a desirable purity of circRNA is achieved from the sample. In embodiments, the percent (w/w) of the circRNA in the enriched population of circRNA is 2% (w/w) of the circRNA in the population of mixed polyribonucleotides in the sample. In embodiments, the percent (w/w) of the circRNA in the enriched population of circRNA is 3% (w/w) of the circRNA in the population of mixed polyribonucleotides in the sample. In embodiments, the percent (w/w) of the circRNA in the enriched population of circRNA is 4% (w/w) of the circRNA in the population of mixed polyribonucleotides in the sample. In embodiments, the percent (w/w) of the circRNA in the enriched population of circRNA is 5% (w/w) of the circRNA in the population of mixed polyribonucleotides in the sample. In embodiments, the percent (w/w) of the circRNA in the enriched population of circRNA is 6% (w/w) of the circRNA in the population of mixed polyribonucleotides in the sample. In embodiments, the percent (w/w) of the circRNA in the enriched population of circRNA is 7% (w/w) of the circRNA in the population of mixed polyribonucleotides in the sample. In embodiments, the percent (w/w) of the circRNA in the enriched population of circRNA is 8% (w/w) of the circRNA in the population of mixed polyribonucleotides in the sample. In embodiments, the percent (w/w) of the circRNA in the enriched population of circRNA is 9% (w/w) of the circRNA in the population of mixed polyribonucleotides in the sample. In embodiments, the percent (w/w) of the circRNA in the enriched population of circRNA is 10% (w/w) of the circRNA in the population of mixed polyribonucleotides in the sample. In embodiments, the percent (w/w) of the circRNA in the enriched population of circRNA is 11% (w/w) of the circRNA in the population of mixed polyribonucleotides in the sample. In embodiments, the percent (w/w) of the circRNA in the enriched population of circRNA is 12% (w/w) of the circRNA in the population of mixed polyribonucleotides in the sample. In embodiments, the percent (w/w) of the circRNA in the enriched population of circRNA is 13% (w/w) of the circRNA in the population of mixed polyribonucleotides in the sample. In embodiments, the percent (w/w) of the circRNA in the enriched population of circRNA is 14% (w/w) of the circRNA in the population of mixed polyribonucleotides in the sample. In embodiments, the percent (w/w) of the circRNA in the enriched population of circRNA is 15% (w/w) of the circRNA in the population of mixed polyribonucleotides in the sample. In embodiments, the percent (w/w) of the circRNA in the enriched population of circRNA is 16% (w/w) of the circRNA in the population of mixed polyribonucleotides in the sample. In embodiments, the percent (w/w) of the circRNA in the enriched population of circRNA is 17% (w/w) of the circRNA in the population of mixed polyribonucleotides in the sample. In embodiments, the percent (w/w) of the circRNA in the enriched population of circRNA is 18% (w/w) of the circRNA in the population of mixed polyribonucleotides in the sample. In embodiments, the percent (w/w) of the circRNA in the enriched population of circRNA is 19% (w/w) of the circRNA in the population of mixed polyribonucleotides in the sample. In embodiments, the percent (w/w) of the circRNA in the enriched population of circRNA is 20% (w/w) of the circRNA in the population of mixed polyribonucleotides in the sample. In embodiments, the percent (w/w) of the circRNA in the enriched population of circRNA is 21% (w/w) of the circRNA in the population of mixed polyribonucleotides in the sample. In embodiments, the percent (w/w) of the circRNA in the enriched population of circRNA is 22% (w/w) of the circRNA in the population of mixed polyribonucleotides in the sample. In embodiments, the percent (w/w) of the circRNA in the enriched population of circRNA is 23% (w/w) of the circRNA in the population of mixed polyribonucleotides in the sample. In embodiments, the percent (w/w) of the circRNA in the enriched population of circRNA is 24% (w/w) of the circRNA in the population of mixed polyribonucleotides in the sample. In embodiments, the percent (w/w) of the circRNA in the enriched population of circRNA is 25% (w/w) of the circRNA in the population of mixed polyribonucleotides in the sample. In embodiments, the percent (w/w) of the circRNA in the enriched population of circRNA is 26% (w/w) of the circRNA in the population of mixed polyribonucleotides in the sample. In embodiments, the percent (w/w) of the circRNA in the enriched population of circRNA is 27% (w/w) of the circRNA in the population of mixed polyribonucleotides in the sample. In embodiments, the percent (w/w) of the circRNA in the enriched population of circRNA is 28% (w/w) of the circRNA in the population of mixed polyribonucleotides in the sample. In embodiments, the percent (w/w) of the circRNA in the enriched population of circRNA is 29% (w/w) of the circRNA in the population of mixed polyribonucleotides in the sample. In embodiments, the percent (w/w) of the circRNA in the enriched population of circRNA is 30% (w/w) of the circRNA in the population of mixed polyribonucleotides in the sample. In embodiments, the percent (w/w) of the circRNA in the enriched population of circRNA is 31% (w/w) of the circRNA in the population of mixed polyribonucleotides in the sample. In embodiments, the percent (w/w) of the circRNA in the enriched population of circRNA is 32% (w/w) of the circRNA in the population of mixed polyribonucleotides in the sample. In embodiments, the percent (w/w) of the circRNA in the enriched population of circRNA is 33% (w/w) of the circRNA in the population of mixed polyribonucleotides in the sample. In embodiments, the percent (w/w) of the circRNA in the enriched population of circRNA is 34% (w/w) of the circRNA in the population of mixed polyribonucleotides in the sample. In embodiments, the percent (w/w) of the circRNA in the enriched population of circRNA is 35% (w/w) of the circRNA in the population of mixed polyribonucleotides in the sample. In embodiments, the enriched population of circRNA has circRNA in a quantity of at least 35% (w/w)) (e.g., at least 36%, 40%, 45%, 50%, 55%, 60%, 65%, 70%, 75%, 80%, 85%, 90%, 95%, 96%, 97%, 98%, 99% or more) and a linRNA quantity that is less than 65% (w/w) (e.g., less than 64%, 60%, 55%, 50%, 45%, 40%, 35%, 30%, 25%, 20%, 15%, 10%, 5%, 4%, 3%, 2%, 1%, or less). For example, the percent (w/w) of the circRNA in the enriched population of circRNA may be 2-fold greater (w/w) of the circRNA in the population of mixed polyribonucleotides in the sample.

### Example 2: Purification of CircRNA with Varying Concentration of Chaotropic Agent and Salt as Denaturing Conditions

The following experiment was performed to assess the ability of varying concentrations of a chaotropic agent, in this case 6 M urea, and a salt, in this case sodium chloride (NaCl) to denature and resolve or further purify circRNA the following experiment was performed.

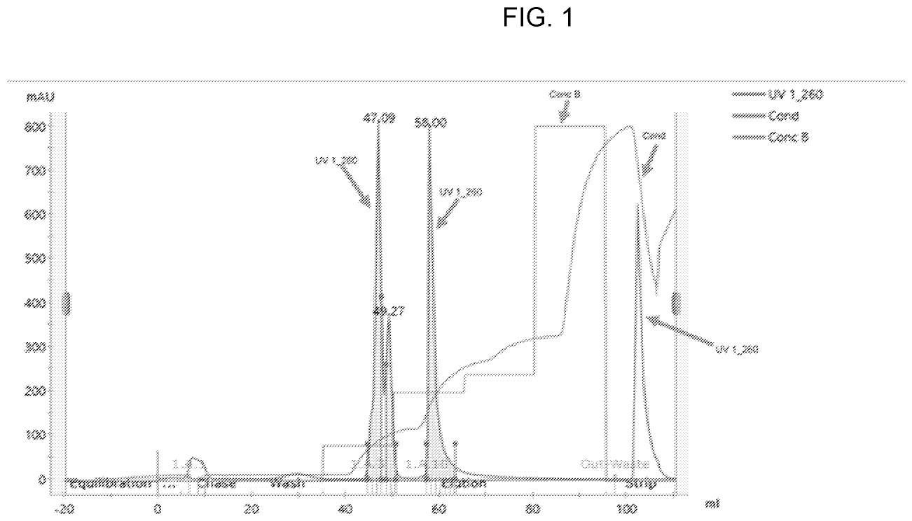

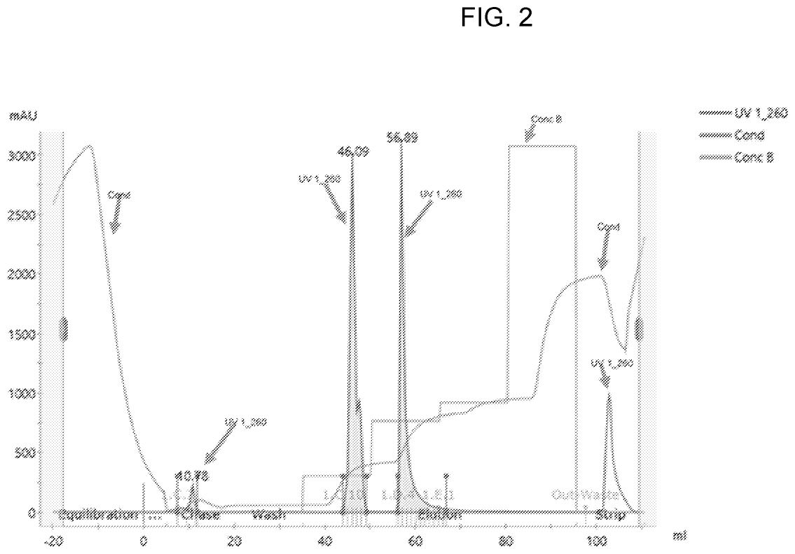

A 5 mL DEAE Sepharose column was used to purify circular RNA generated by splint ligation. The column was equilibrated and washed with 50 mM Tris, pH 7 and loaded with circular RNA. The column was either eluted with step elutions at 300 mM NaCl, 700 mM NaCl, 1 M NaCl, 3 M NaCl or either step elutions with urea at 300 mM NaCl+0.6 M urea, 700 mM NaCl+1.5 M urea, 1 M NaCl+1.8 M urea and 3 M NaCl+6 M urea. Relevant fractions were pooled and resulting enriched material was analyzed by PAGE. Data is shown below in Table 2. A chromatogram with no urea present is shown in FIG. 1. The peak denoted at 58.00 mL is the circRNA and all other peaks are impurities. A chromatogram with urea present is shown in FIG. 2, representing the step elutions present with urea. The peak denoted at 56.89 mL is the circRNA and all other peaks are impurities.

TABLE 2

| Gel/Lane Number | Sample | % circRNA | % linRNA |
|---|---|---|---|
| Gel 1/Lane 1 | Input (or IVT) | 44 | 55 |
| Gel 2/Lane 1 | + Urea | 66 | 34 |
| Gel 2/Lane 8 | − Urea | 56 | 43 |

Prior to both purifications the starting material purity was 44.6% and 55.4% for circRNA and linRNA percentages respectively measured by PAGE. In absence of urea the circRNA percent purity was 56.4% and the linear was 43.6%. The addition of 6 M urea resulted in 65.9% and 34.1% purity for circRNA and linRNA respectively. To develop a scalable process 1 mL-8 L column volume or larger the following purification was performed to understand the purity improvements of urea at constant concentration.

### Example 3: Purification of CircRNA with Chaotropic Agent and High Salt Denaturing Conditions

The following experiment was performed to assess the ability of varying concentrations of a chaotropic agent, in this case 6 M urea, and a salt, in this case sodium chloride (NaCl) to denature and resolve or further purify circRNA the following experiment was performed.

To purify circular RNA an 8 mL DEAE monolithic column was used in the presence of 6 M urea.

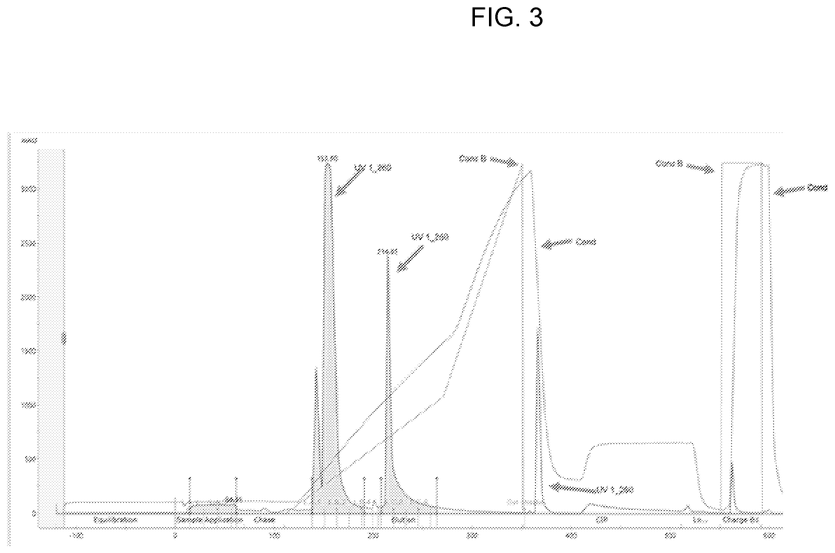

The experiment was performed on about a 200 mg/mL not cleaned up post ligation of circular RNA at a pH of 7. The sample was diluted with 1:1 with sample buffer (100 mM Tris-HCl PH 7.0, 6 M urea, 10 mM EDTA) to a final concentration of 3M urea and 7.5 mg was loaded onto the 8 mL column. The column was equilibrated and washed after sample load with 50 mM Tris-HCl PH 7.0, 6 M urea, 5 mM EDTA and eluted with a 20CV linear gradient to 100% of 50 mM Tris-HCL pH 7.0, 6 M Urea, 1 M NaCl 5 mM EDTA followed by at 10 CV linear gradient to 100% of 50 mM Tris-HCL pH 7.0, 6 M Urea, 3M NaCl, 5 mM EDTA. The column was cleaned with 1 M NaOH prior to re-equilibration. The chromatogram, as shown in FIG. 3, was evaluated and the peaks pooled. By PAGE 200 ng was quantitated for purity compared to the loaded material and displayed below in Table 3.

TABLE 3

| Sample | % circRNA | % linRNA |
|---|---|---|
| Starting Material | 47 | 53 |
| circRNA Elution Peak | 58 | 42 |

The starting material circRNA percent purity by PAGE is 47% and after purification using the conditions described directly above, the circRNA purity percent is 58. %.

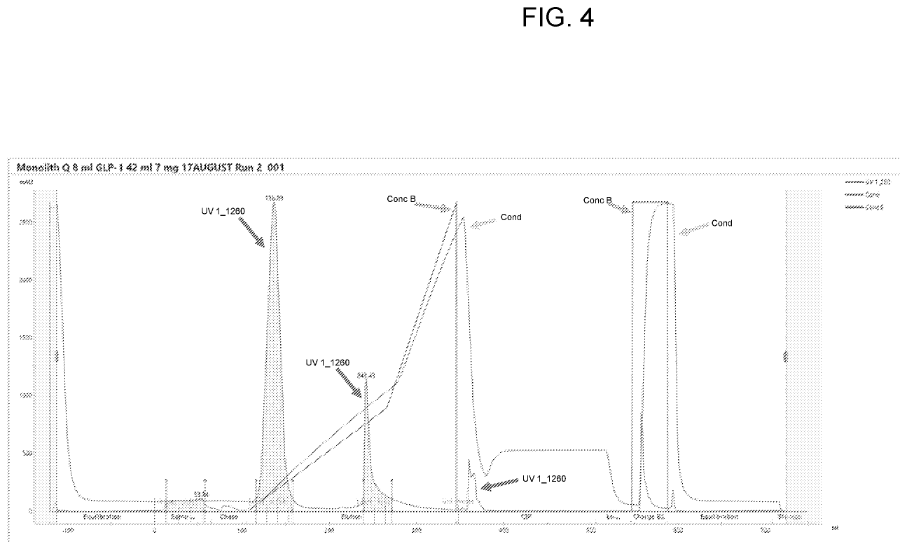

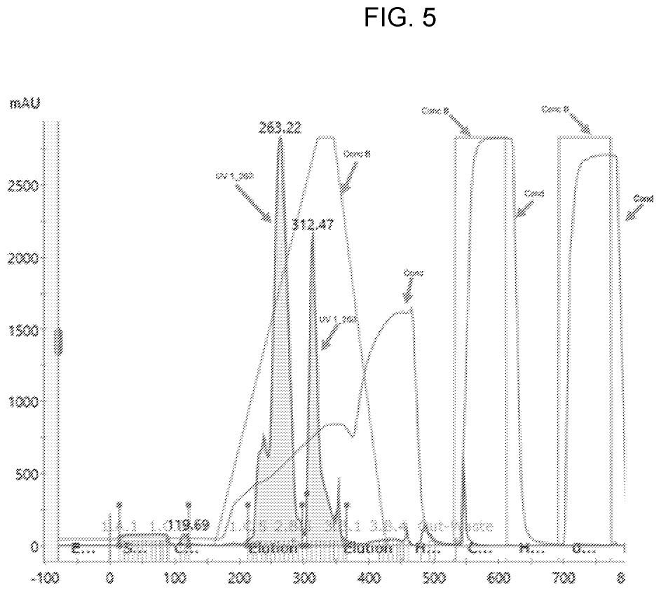

The same experimental conditions as described directly above were performed for QA 8 mL Monolithic column from BIA separations except the pH of the buffers was pH 8.0. The purity measured by PAGE was recorded and is shown in Table 4. FIG. 4 shows the purification of 7 mg of circRNA IVT starting material. In FIG. 5 peaks at 136.89 mL are impurities and the circRNA peak is denoted in the chromatogram at 242.43 mL respectively.

TABLE 4

| Sample | Fractions | % circRNA | % linRNA |
|---|---|---|---|
| Starting Material | NA | 49 | 51 |
| circRNA Elution Peak | B4 | 62 | 38 |
| circRNA Elution Peak | B5 | 64 | 36 |

The input starting material or load is 49% circRNA quantitated by 6PAGE. The circRNA eluted within fraction B4 is 62% pure for circRNA and 64% pure for faction B5.

### Example 4: Purification of CircRNA with High Salt, High pH, Chelating Agent, and Chaotropic Agent Denaturing Conditions

To assess the ability of two or more denaturing conditions to denature and resolve or further purify circRNA the following experiment was performed.

Diluted 2 mL at 28 mg/mL (˜56 mg by Quibit) of circular RNA post in vitro transcription to 9 mL with water then diluted with 1:1 sample buffer (pH 7.0 100 mM Tris-HCl, 6 M urea, 10 mM EDTA) to 18 mL. The sample was loaded onto an 8 mL column with hydrogen bonding and anion exchange. The column was equilibrated and washed with 50 mM bicarbonate pH 7.0, 6 M urea, 5 mM EDTA and a 20 CV linear gradient was applied to 100% of 50 mM bicarbonate pH 10.0, 6 M urea, 5 mM EDTA, 1 M NaCl followed by a 10 CV linear gradient to 100% of 50 mM Tris-HCl pH 10.0, 6 M urea, 5 mM EDTA, 3M NaCl. The column was cleaned with 0.1 M NaOH 3M NaCl and 0.1 M acetic acid 3M NaCl prior to re-equilibration. Chromatogram results are displayed in FIG. 5.

TABLE 5

| Sample | % circRNA | % linRNA |
|---|---|---|
| Starting Material | 60 | 37 |
| circRNA Elution Peak | 63 | 35 |

The load circRNA % is 60% and 37% for linear by PAGE quantification. The peak at 263.22 mL contained 62.91% circRNA and 35.19% linRNA purity by PAGE. All other peaks are impurities.

### Example 5: Purification of CircRNA with High Salt, Chelating Agent, Temperature and Chaotropic Agent Denaturing Conditions

To assess the ability of two or more denaturing conditions to denature and resolve or further purify circRNA the following experiment was performed using high salt, chelating agent, temperature, and chaotropic agent, in this case urea, as denaturing conditions.

An experiment was conducted on a sample containing a mixed population of polyribonucleotides containing circRNA, linRNA, or other impurities or by-products (e.g., impurities or by-products described herein) using HIC purification. Purification was done in the presence 3 M NaCl in the sample followed by elution by 3 M NaCl, 6 M urea immediately followed by an additional step elution at 0 M NaCl, 6 M urea immediately followed by 0 M NaCl, 0 M urea in sequential order during the same run on the same column and sample load all containing base buffer of 50 mM sodium phosphate, pH 7.0, 5 mM EDTA. The following conditions were tested for 2.17 mg/ml (column volume) circular RNA in the presence of 3 M NaCl, 50 mM sodium phosphate, pH 7.0, 5 mM EDTA: room temperature, 50° C., 60° C. and 75° C. for 10 min each prior to loading the sample at room temperature onto a 1 mL butyl high ligand density (HLD) HIC column.

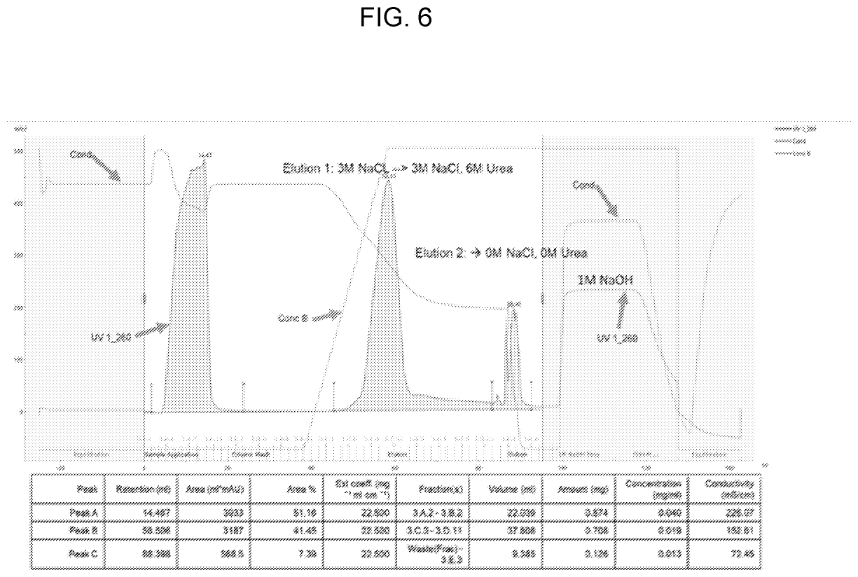

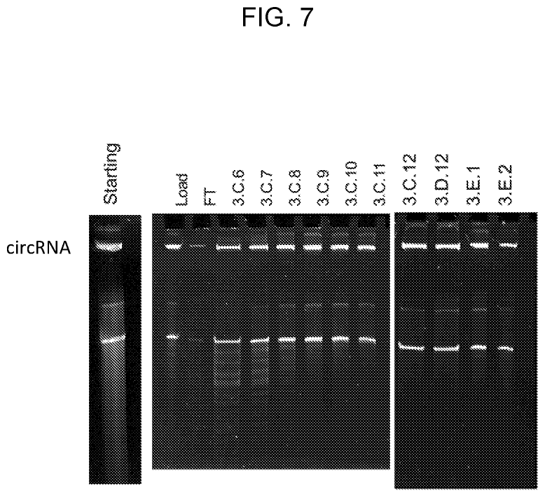

FIG. 6, FIG. 7, and Table 6 show the results following the purification of the circular RNA using the 1 mL C4 HLD column where the sample was incubated for 10 min at 20C as stated above and loaded at 2.17 mg onto the column.

TABLE 6

| Sample | Fraction | % circRNA |
|---|---|---|
| Starting Material | | 61.5 |
| Flow Through at 14.47 ml | 3.A.9 | na |
| ELUTION Peak at 58.51 ml | 3.C.6 | 58.7 |
| | 3.C.7 | 58 |
| | 3.C.8 | 58.2 |
| | 3.C.9 | 59.7 |
| | 3.C.10 | 60.5 |
| | 3.C.11 | 64.4 |
| | 3.C.12 | 71.2 |
| Small Peak at 84 ml | 3.D.12 | 64.6 |
| Elution Peak at 88.40 ml | 3.E.1 | 63.7 |
| | 3.E.2 | 60.4 |

The input starting material for the first gel was 61.5% circRNA. The flow through (FT) contained very little RNA and the majority of the RNA bound the column. The first elution peak at 58.52 mL including fractions 3.C.6-3.C.12 had circRNA purity range from 58.0-71.2% circRNA purity by PAGE, depending on which fraction was tested. The second elution peak at 88.40 mL including fractions 3.E.1 and 3.E.2 contained 63.7% and 60.4% circRNA purity by PAGE respectively.

### Example 6: Purification of CircRNA with High Temperature, Chaotropic Agent, Organic Solvent, and High Salt Denaturing Conditions

To assess the ability of two or more denaturing conditions to denature and resolve or further purify circRNA the following experiment was performed.

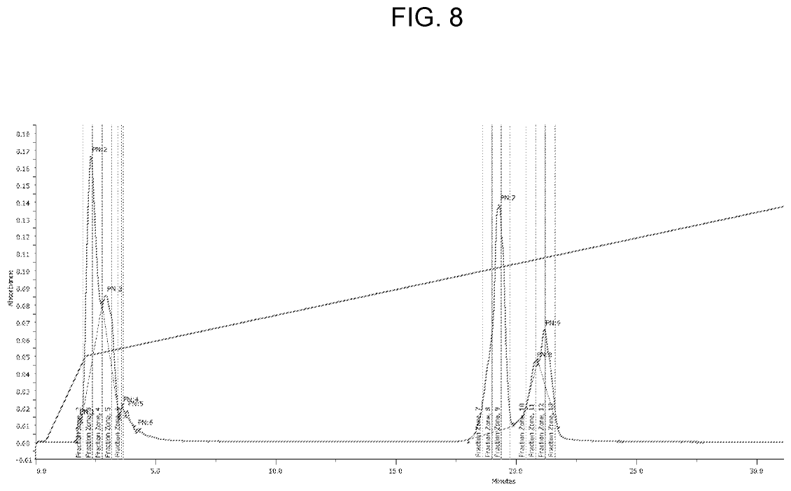

A 10 mg sample of IVT RNA containing a mixture of circRNA and linRNA was loaded onto an anion exchange oligonucleotide column and the entire purification was performed at 80° C. using an in-line flow heater. The column conditions for the equilibration and wash are 20 mM bis-tris propane, 6 M urea, 20% ACN, pH 7.0 and the sample was eluted by a 30%-60% and a 6 CV linear gradient to 20 mM Bis-tris Propane, 6 M urea, 1 M NaCl, 20% ACN, pH 7.0. The chromatogram in FIG. 8 was collected and the circRNA was denoted as PN: 7 and the linear RNA was denoted as PN: 8 and PN: 9. Results are shown in Table 7.

TABLE 7

| Fraction | Yield (mg) | % circRNA Purity |
|---|---|---|
| 7 | 0.5 | 57 |
| 8 | 1.5 | 96 |
| 9 | 0.9 | 94 |
| 10 | 0.4 | 8 |
| 11 | 0.7 | 2 |
| 12 | 0.6 | 2 |
| Total | 4.6 | |

Fractions 7, 8 and 9 containing the circRNA yielded 0.5 mg, 1.5 mg and 0.9 mg of material at 57%, 96%, and 94% circRNA purity respectively by the HPLC analytical method. The linRNA peak including fractions 10, 11, and 12 contained 0.4, 0.7, and 0.6 mg of material at 8%, 2% and 2% circRNA purity respectively by the HPLC analytical method.

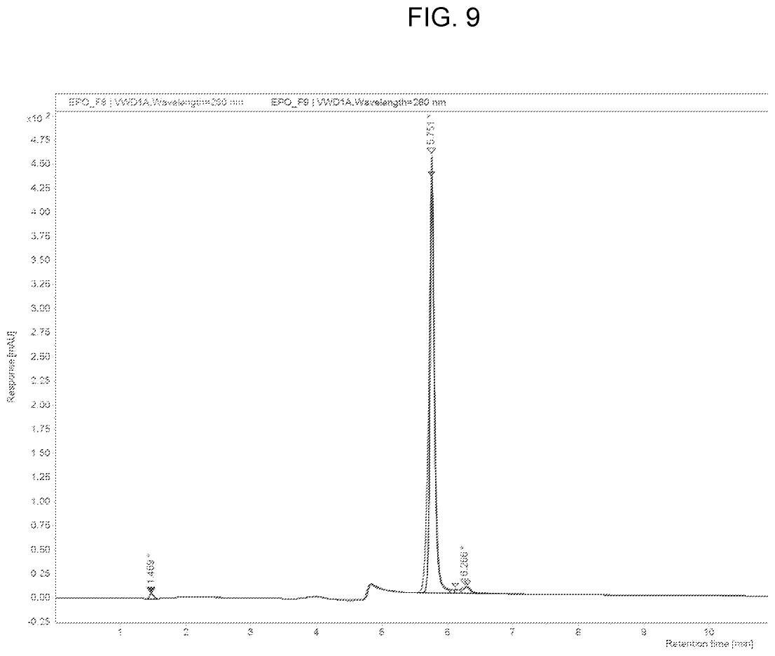

Fraction 8 and 9 HPLC analytical results are shown in FIG. 9.

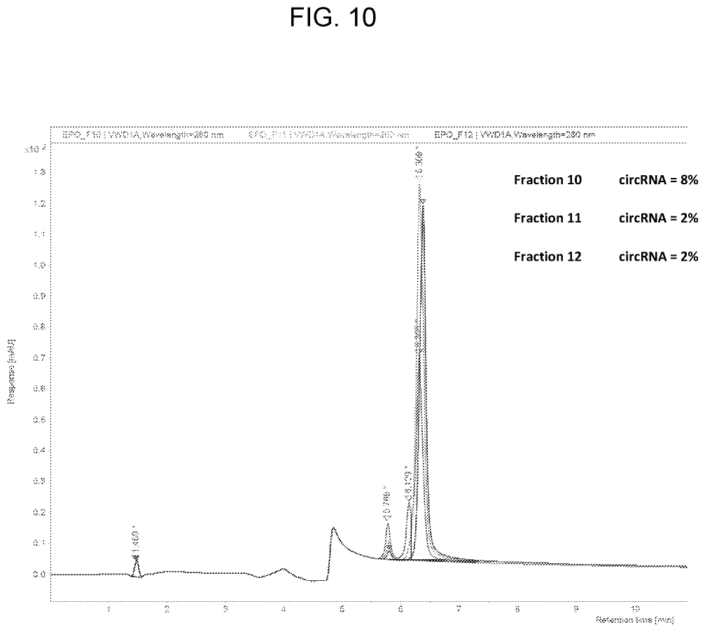

Fraction 10, 11, and 12 HPLC analytical results are shown in FIG. 10.

### Example 7: Purification of CircRNA with Varying Temperature, Chelating Agent, and Chaotropic Agent Denaturing Conditions

The present Example demonstrates the use of temperature, chelating agent, and chaotropic agent, as denaturing conditions in the separation of circRNA from linRNA, and other impurities or by-products where 0.5 M Guanidine-HCl is present throughout the experiment(s).

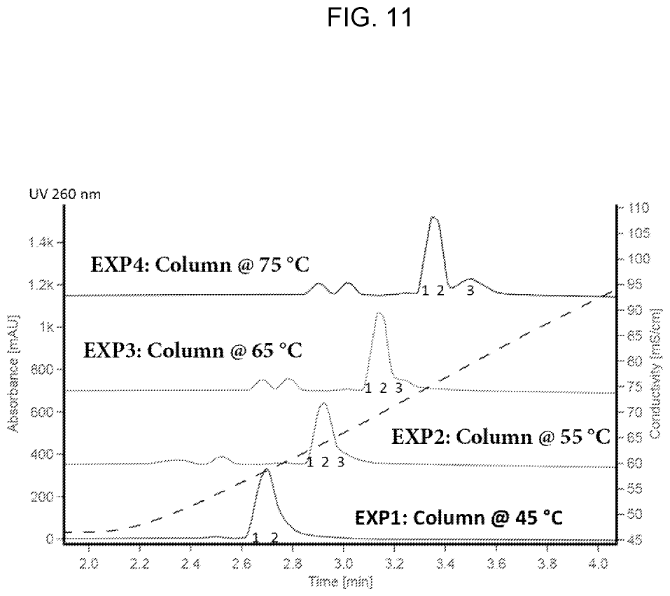

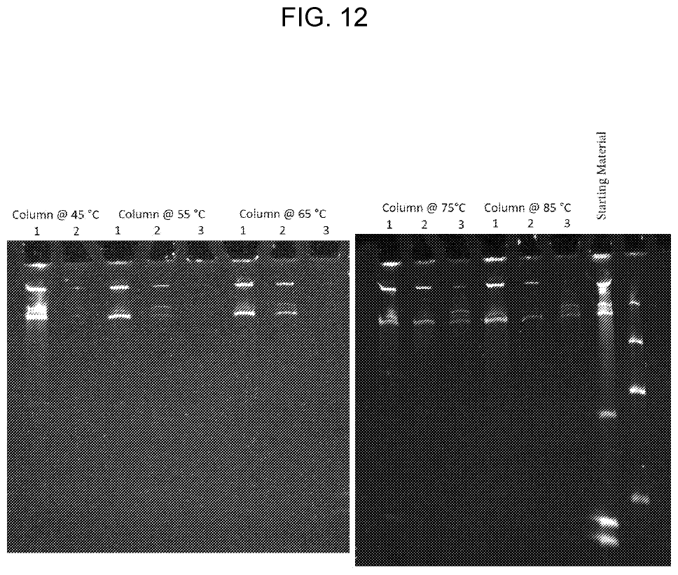

An 0.1 ml QA Monolithic Column was used and IVT generated circRNA sample within an auto sampler at 5° C. was injected onto the column that was equilibrated with 50 mM Tris, 6 M urea, 5 mM EDTA, pH 8. The column was eluted with a linear gradient to 100% 50 mM Tris, 6 M urea, 5 mM EDTA, 1 M NaCl, pH 8 and run at 1 ml/min at 45° C., 55° C., 65° C., 75° C. and 85° C. (85° C. chromatographic curve not shown in figure) respectively as shown in FIG. 11 and FIG. 12. Each number displayed within the chromatogram corresponds to the sample loaded onto the PAGE for analysis.

### Example 8: Purification of CircRNA with High Temperature and Organic Solvent Denaturing Conditions

To assess the ability of two or more denaturing conditions to denature and resolve or further purify circRNA the following experiment was performed.

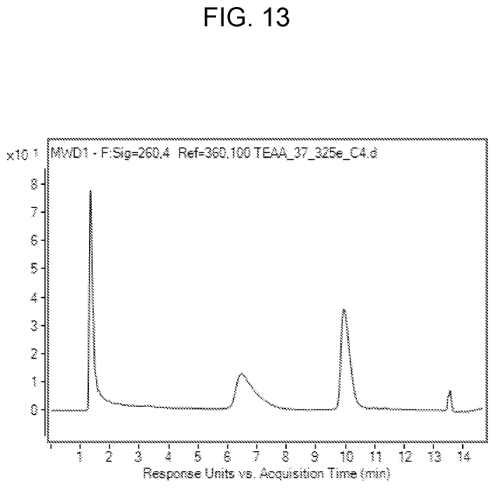

In this method, ion-pair reverse phase HPLC was used to separate circRNA and linRNA using temperature and organic solvent denaturing conditions. A sample of circRNA/linRNA was injected onto an anion exchange oligonucleotide column (e.g., DNAPac PA200 oligonucleotide column). The initial chromatography conditions were 53% Mobile Phase A (100 mM TEAA in water) and 47% Mobile Phase B (100 mM TEAA in 25% acetonitrile in water). The column was heated to 75° C. during the run. Elution of the circRNA and linRNA was carried out according to Table 8. FIG. 13 shows exemplary chromatogram results.

TABLE 8

| Time (minutes) | % Mobile Phase A | % Mobile Phase B |
|---|---|---|
| 3.00 | 53.0 | 47.0 |
| 3.50 | 53.0 | 47.0 |
| 10.00 | 51.0 | 49.0 |
| 10.50 | 0.0 | 100.0 |
| 11.50 | 0.0 | 100.0 |
| 12.00 | 53.0 | 47.0 |
| 20.00 | 53.0 | 47.0 |

### Example 9: Purification of CircRNA with Varying pH Denaturing Conditions

The present Example describes the use of pH as denaturing conditions in the separation of circRNA from linRNA, and other impurities or by-products. Denaturing pH conditions of less than 5 (e.g., 4.5, 4, 3.5, 3, 2.5, 2, 1.5, 1, or less than 1) or greater than 9 (e.g., 9.5, 10, 10.5, 11, 11.5, 12, 12.5, 13, or greater than 13) are tested.

### Example 10: Purification of CircRNA with Salt and Varying pH Denaturing Conditions

The present Example describes the use of varying pH and salt solution denaturing conditions in the separation of circRNA from linRNA, and other impurities or by-products.

Salts to be tested are NaCl, KCl, MgCl, CaCl, CsSO₄, NaSO₄, LiCl, and/or LiBr. Denaturing salt solution concentrations between 100 mM and 1 M are tested with denaturing pH conditions of less than 5 (e.g., 4.5, 4, 3.5, 3, 2.5, 2, 1.5, 1, or less than 1) or greater than 9 (e.g., 9.5, 10, 10.5, 11, 11.5, 12, 12.5, 13, or greater than 13). The salt solutions and/or pH conditions to be tested are added in wash steps, added into the sample directly prior to purification or added into the equilibration buffer or elution buffer.

### Example 11: Purification of CircRNA with Organic Solvent Denaturing Conditions

The present Example describes the use of organic solvents as denaturing conditions in the separation of circRNA from linRNA, and other impurities or by-products. Denaturing organic solvent concentrations of at least 10% (v/v) are tested. Organic solvents tested include dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, and/or propylene glycol. The organic solvents to be tested are added in wash steps, added into the sample directly prior to purification or added into the equilibration buffer or elution buffer.

### Example 12: Purification of CircRNA with Detergent Denaturing Conditions

The present Example describes the use of detergents as denaturing conditions in the separation of circRNA from linRNA, and other impurities or by-products. Detergents to be tested include Nonidet P-40 (NP40), a polysorbate (e.g., Tween-20, Tween-40, Tween-60, or Tween-80), Triton X 100, CHAPS, octyl β-D-glucopyranoside, and/or n-dodecyl β-maltoside, at concentrations ranging from 0.00001% to 20%. The detergents to be tested are added during wash steps, added into the sample directly prior to purification or added into the equilibration buffer or elution buffer.

### Example 13: Analytical Method Under Denaturing Conditions

The present Example demonstrates the use of denaturing conditions in analytical methods.

An analytical anion-exchange (AEX) HPLC-UV method was demonstrated to be capable of resolving circRNA from linRNA, and thus is suitable for determining the purity of circRNA samples in the presence of linRNA.

In this method, a circRNA sample was injected onto an anion exchange oligonucleotide column (e.g., DNAPac PA200 oligonucleotide column). The initial chromatography conditions were 85% Mobile Phase A (20 mM Bis-Tris, 6 M urea, pH 7.0) and 15% Mobile Phase B (20 mM Bis-Tris, 6 M urea, 1 M NaCl, pH 7.0). The column was heated to 80° C. during the run. Elution of the circRNA and linRNA was carried out using a gradient up to 10% Mobile Phase A/90% Mobile Phase B.

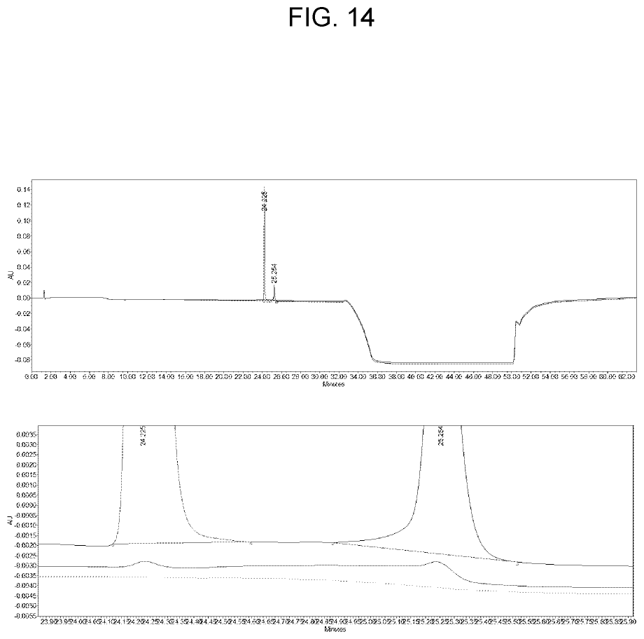

FIG. 14 shows exemplary chromatograms of circRNA at a concentration of 0.2223 mg/mL in water with two following water injections.

### Example 14: Analytical Method Under Denaturing Conditions

The present Example demonstrates the use of denaturing conditions in analytical methods.

The analytical method described above in Example 9 was further modified with an addition of the organic denaturant acetonitrile at a concentration of 20% to the mobile phase. Other modifications include a change of buffer salt to improve the buffering capacity of the mobile phase.

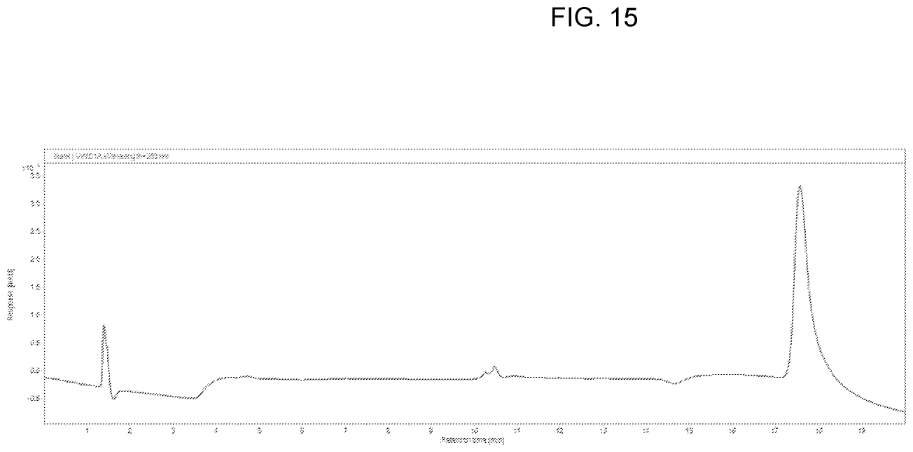

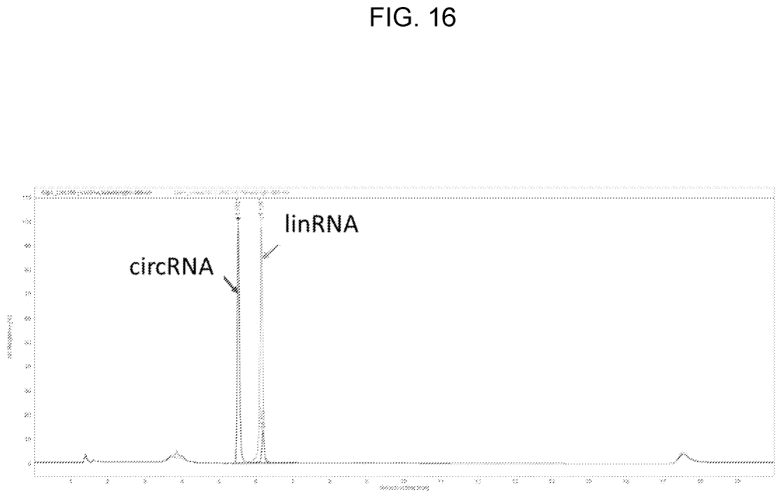

Specifically, a circRNA sample was injected onto an anion exchange oligonucleotide column. The sample was prepared by dilution with a denaturing agent (e.g., DMSO) or directed diluted into Mobile Phase A (20 mM bis-tris propane, 6 M urea, pH 7.0) prior to injection. The initial chromatography conditions were 85% Mobile Phase A and 15% Mobile Phase B (20 mM bis-tris propane, 6 M urea, 1 M NaCl, pH 7.0). The column was heated to 80° C. during the run. Elution of the circRNA and linRNA was carried out using a gradient up to 10% Mobile Phase A (20 mM Bis-Tris, 6 M urea, pH 7.0) and 90% Mobile Phase B (20 mM Bis-Tris, 6 M urea, 1 M NaCl, pH 7.0). FIG. 15 shows the chromatogram for a blank injection. CircRNA and linRNA standard gave separate peaks as shown in FIG. 16.

### OTHER EMBODIMENTS

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features set forth herein.

1. A method for producing an enriched population of circular polyribonucleotides (circRNA), the method comprising:
(a) providing a sample comprising a population of polyribonucleotides comprising circRNA and linear polyribonucleotides (linRNA); and
(b) separating the circRNA from the linRNA under denaturing conditions that do not comprise the use of gel electrophoresis, thereby producing an enriched population of circRNA comprising a length of 1,000 nucleotides or less.

2. A method for producing an enriched population of circRNA, the method comprising:
(a) providing a sample comprising a population of polyribonucleotides comprising circRNA and linRNA, wherein the circRNA comprise a length of 1,000 nucleotides or less; and
(b) exposing the population of polyribonucleotides to denaturing conditions, thereby enriching the population of circRNA.

3. The method of claim 1 or 2, wherein:
(i) the total weight of polyribonucleotides in the population of polyribonucleotides is at least 1 μg;
(ii) the total volume of the sample comprising the population of polyribonucleotides is at least 500 μL; or
(iii) the concentration of the population of polyribonucleotides in the sample is at least 200 ng/μL.

4. The method of claim 2 or 3, wherein the separating step (b) is performed under denaturing conditions that do not comprise the use of gel electrophoresis; and/or wherein the enriched population of circRNA is substantially free of one or more impurities or by-products, wherein the one or more impurities or by-products comprise polyacrylamide, boric acid, magnesium, or ethylenediaminetetraacetic acid (EDTA).

5. The method of any one of claims 1-4, wherein the denaturing conditions comprise a temperature of at least 50° C.; or wherein the denaturing conditions comprise a temperature of at least 50° C. followed by a temperature of not greater than 8° C. within a time period of no greater than 30 seconds.

6. The method of any one of claims 1-5, wherein the denaturing conditions comprise a pH of less than 5 or greater than 9.

7. The method of any one of claims 1-6, wherein the denaturing conditions comprise a chemical treatment comprising treatment with an acid, base, organic solvent, chaotropic agent, crowding agent, chelator, detergent, or salt solution.

8. The method of claim 7, wherein the acid comprises between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

9. The method of claim 7 or 8, wherein the base comprises between 1 mM and 500 mM sodium hydroxide, potassium hydroxide, imidazole, histidine, sodium bicarbonate, guanidine, or triethylamine.

10. The method of any one of claims 7-9, wherein the organic solvent comprises at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

11. The method of any one of claims 7-10, wherein the chaotropic agent comprises between 100 mM and 8 M urea, guanidinium chloride, lithium perchlorate, or polyethylene glycol (PEG); and/or wherein the chaotropic agent comprises between 100 mM and 8 M n-dodecyl β-d-maltoside, n-octylglucoside, CHAPS, or deoxycholate.

12. The method of any one of claims 7-11, wherein the crowding agent comprises between 100 mM and 8 M PEG, or urea.

13. The method of any one of claims 7-12, wherein the chelator comprises between 1 mM and 10 mM ethylene glycol-bis(β-aminoethyl ether)-N,N,N, N-tetra acetic acid (EGTA) or derivatives thereof, ethylenediaminetetraacetic acid (EDTA) or derivatives thereof, nitrilotriacetic acid (NTA), iminodisuccinic acid (IDS), polyaspartic acid, S,S-ethylenediamine-N,N′-disuccinic acid (EDDS), or methylglycinediacetic acid (MGDA).

14. The method of any one of claims 7-13, wherein the detergent comprises between 0.005% and 0.05% (v/v) Nonidet P-40 (NP40), CHAPS, octyl β-D-glucopyranoside, n-dodecyl β-d-maltoside, Tween-20, or Tween-80.

15. The method of any one of claims 1-14, wherein step (b) comprises performing column chromatography on the population of polyribonucleotides, wherein performing column chromatography comprises an equilibration step, sample loading step, column washing step, and elution step; wherein the separating is performed during the sample loading step, the column washing step, and/or during the elution step.

16. The method of claim 15, wherein the column chromatography comprises fast protein liquid chromatography (FPLC), high-pressure liquid chromatography (HPLC), hydrophobic interaction chromatography (HIC), anion exchange chromatography (AEC), mixed mode chromatography, or affinity chromatography.

17. The method of claim 16, wherein the AEC comprises
(i) use of an anion exchange resin, wherein the anion exchange resin is selected from the group consisting of styrene-divinylbenzene, silica, sepharose, cellulose, dextran, epoxy polyamine, methacrylate, agarose, and acrylic; and/or wherein the anion exchange resin comprises an ion exchanger selected from the group consisting of quaternary ammonium, amino ethyl, diethylaminoethyl, and diethylaminopropyl; and/or wherein the anion exchange resin comprises beads, wherein the beads have a bead diameter of 45-165 μm and a pore size of diameter 100-1000 nm; and/or
(ii) use of a linear gradient elution or a step isocratic elution; and/or
(iii) use of a flow rate that is between 1 mL/min and 150 mL/min.

18. The method of any one of claims 1-17, wherein step (b) is performed by pooling multiple fractions of purified circRNA.

19. The method of any one of claims 1-18, wherein the circRNA and the linRNA have the same ribonucleotides sequence; and/or wherein the circRNA and the linRNA have the same mass; and/or wherein the circRNA and the linRNA lack a poly(A) tail.

20. The method of any one of claims 1-19, wherein the method comprises exonuclease digestion of the linRNA or wherein the method does not comprise exonuclease digestion of the linRNA; and/or wherein the method does not comprise a selective modification to the circRNA or to the linRNA that improves enrichment of the circRNA.

21. The method of any one of claims 1-20, wherein the percent (w/w) of the circRNA in the enriched population of circRNA is 2-fold greater than the percent (w/w) of the circRNA in the polyribonucleotide population; and/or wherein the percent (w/w) of the circRNA in the enriched population of circRNA is at least 65%; and/or wherein the percent (w/w) of the linRNA in the enriched population of circRNA is less than 35%.

22. A composition comprising a population of polyribonucleotides comprising circRNA and linRNA, wherein the circRNA comprise a length of 1,000 nucleotides or less, wherein the population of polyribonucleotides is in solution under denaturing conditions, and wherein:
(i) the total mass of polyribonucleotides in the population of polyribonucleotides is at least 1 μg;
(ii) the total volume of the sample comprising the population of polyribonucleotides is at least 500 μL; or
(iii) the concentration of the population of polyribonucleotides in the sample is at least 500 ng/μL.

23. A composition comprising an enriched population of circRNA, wherein:
(a) the composition is obtained from a sample comprising a population of polyribonucleotides comprising circRNA and linRNA, wherein the circRNA comprise a length of 1,000 nucleotides or less;
(b) the composition has been exposed to one or more denaturing conditions; and
(c) the composition is substantially free of one or more impurities or by-products.

24. The composition of claim 22 or 23, wherein the one or more impurities or by-products comprise polyacrylamide, boric acid, magnesium, or EDTA.

25. The composition of any one of claims 22-24, wherein the denaturing conditions comprise a temperature of at least 50° C.; or wherein the denaturing conditions comprise a temperature of at least 50° C. followed by a temperature of not greater than 8° C.

26. The composition of any one of claims 22-25, wherein the denaturing conditions comprise a pH of less than 5 or greater than 9.

27. The composition of any one of claims 22-26, wherein the denaturing conditions comprise a chemical treatment comprising treatment with an acid, base, organic solvent, chaotropic agent, crowding agent, chelator, detergent, or salt solution.

28. The composition of claim 27, wherein the acid comprises between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

29. The composition of claim 27 or 28, wherein the base comprises between 1 mM and 500 mM sodium hydroxide, potassium hydroxide, imidazole, histidine, sodium bicarbonate, guanidine, or triethylamine.

30. The composition of any one of claims 27-29, wherein the organic solvent comprises at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

31. The composition of any one of claims 27-30, wherein the chaotropic agent comprises between 100 mM and 8 M of urea, guanidinium chloride, lithium perchlorate, or polyethylene glycol (PEG); and/or wherein the chaotropic agent comprises between 100 mM and 8 M of n-dodecyl β-d-maltoside, n-octylglucoside, CHAPS, or deoxycholate.

32. The method of any one of claims 27-31, wherein the crowding agent comprises between 100 mM and 8 M polyethylene glycol (PEG), or urea.

33. The composition of any one of claims 27-32, wherein the chelator comprises between 1 mM and 10 mM EGTA or derivatives thereof, EDTA or derivatives thereof, NTA, IDS, EDDS, or MGDA.

34. The composition of any one of claims 27-33, wherein the detergent comprises between 0.005% and 0.05% (v/v) NP40, CHAPS, octyl β-D-glucopyranoside, n-dodecyl β-d-maltoside, Tween-20, or Tween-80.

35. The composition of any one of claims 22-34, wherein the percent (w/w) of the circRNA in the enriched population of circRNA is 2-fold greater than the percent (w/w) of the circRNA in the polyribonucleotide population; and/or wherein the percent (w/w) of the circRNA in the enriched population of circRNA is at least 65%; and/or wherein the percent (w/w) of the linRNA in the enriched population of circRNA is less than 35%.

36. The composition of any one of claims 22-35, wherein the circRNA and the linRNA have the same ribonucleotides sequence; and/or wherein the circRNA and the linRNA have the same mass; and/or wherein the circRNA and the linRNA lack a poly(A) tail.

37. A method of determining the purity of a circRNA, the method comprising:
(a) providing a sample comprising a population of polyribonucleotides comprising circRNA and linRNA, wherein the circRNA comprise a length of 1,000 nucleotides or less;
(b) separating the circRNA from the linRNA under denaturing conditions by chromatography; and
(c) collecting a chromatogram of the sample comprising a peak for the circRNA and a peak for the linRNA;
(d) calculating the area under each peak to determine the purity of the circRNA in the sample.

38. The method of claim 37, wherein the denaturing conditions do not comprise the use of gel electrophoresis.

39. The method of claim 37 or 38, wherein the denaturing conditions comprise a temperature of at least 50° C.; or wherein the denaturing conditions comprise a temperature of at least 50° C. followed by a temperature of not greater than 8° C. within a time period of no greater than 30 seconds.

40. The method of any one of claims 37-39, wherein the denaturing conditions comprise a pH of less than 5 or greater than 9.

41. The method of any one of claims 37-40, wherein the denaturing conditions comprise a chemical treatment comprising treatment with an acid, base, organic solvent, chaotropic agent, crowding agent, chelator, detergent, or salt solution.

42. The method of claim 41, wherein the acid comprises between 1 mM and 500 mM acetic acid, hydrochloric acid, salicylic acid, phosphoric acid, boric acid, formic acid, oxalic acid, citric acid, benzoic acid, monochloroacetic acid, dichloroacetic acid, trichloroacetic acid, ascorbic acid, or nitric acid.

43. The method of claim 41 or 42, wherein the base comprises between 1 mM and 500 mM sodium hydroxide, potassium hydroxide, imidazole, histidine, sodium bicarbonate, guanidine, or triethylamine.

44. The method of any one of claims 41-43, wherein the organic solvent comprises at least 0.1% (v/v) of dimethyl sulfoxide, triethylammonium acetate, methanol, ethanol, 2-propanol, isopropanol, 1-butanol, 2-butanol, t-butanol, isobutanol, phenol, chloroform, hexane, acetonitrile, formamide, acetone, denatonium, or propylene glycol.

45. The method of any one of claims 41-44, wherein the chaotropic agent comprises between 100 mM and 8 M urea, guanidinium chloride, lithium perchlorate, or polyethylene glycol (PEG); and/or wherein the chaotropic agent comprises between 100 mM and 8 M n-dodecyl β-d-maltoside, n-octylglucoside, CHAPS, or deoxycholate.

46. The method of any one of claims 41-45, wherein the crowding agent comprises between 100 mM and 8 M polyethylene glycol (PEG), or urea.

47. The method of any one of claims 41-46, wherein the chelator comprises between 1 mM and 10 mM EGTA or derivatives thereof, EDTA or derivatives thereof, NTA, IDS, polyaspartic acid, EDDS, or MGDA.

48. The method of any one of claims 41-47, wherein the detergent comprises between 0.005% and 0.05% (v/V) Nonidet P-40 (NP40), CHAPS, octyl β-D-glucopyranoside, n-dodecyl β-d-maltoside, Tween-20, or Tween-80.

49. The method of any one of claims 37-48, wherein the chromatography is liquid chromatography, wherein the liquid chromatography is selected from the group consisting of FPLC, HPLC, HIC, AEC, MMC, or affinity chromatography.

50. The method of any of claims 37-49, wherein the relative standard deviation (RSD) of the purity is less than 5%.
